(19) United States
(12) Patent Application Publication
(10) Pub. No.: US 2012/0040383 A1
(43) Pub. Date: Feb. 16, 2012

(54) Methods and Kits Relating To Metabolite Biomarkers For Colorectal Cancer

(76) Inventors: Wei Jia, Concord, NC (US); Yunping Qiu, Kannapolis, NC (US)

(21) Appl. No.: 13/209,125

(22) Filed: Aug. 12, 2011

Related U.S. Application Data
(60) Provisional application No. 61/373,075, filed on Aug. 12, 2010.

(51) Int. Cl. C12Q 1/58; C12M 1/34; G01N 33/48; G01N 27/00

(52) U.S. Cl. 435/12; 436/64; 436/90; 435/287.1; 422/68.1

(57) ABSTRACT

Embodiments of the present invention provide methods of assessing metabolite biomarkers to diagnose, monitor, prognose and treat colorectal cancer, as well as kits and systems in use thereof. In some embodiments, the metabolite biomarker profile of an subject suspected of having colorectal cancer may be compared to the metabolite biomarker profile of a healthy subject to determine if there are differences between the profiles indicative of colorectal cancer. In some embodiments, the stage of a subject's colorectal cancer may be determined. In certain embodiments, the metabolite biomarker profile of an subject may be useful in determining method of treatment for colorectal cancer. In other embodiments, the metabolite biomarker profiles of an subject having colorectal cancer may be compared prior to and after treatment to determine if there are differences between the profiles indicative of decreased colorectal cancer morbidity.

### CROSS-REFERENCE TO RELATED APPLICATIONS

The present application claims priority under 35 U.S.C. §119(e) from U.S. Provisional Patent Application Ser. No. 61/373,075, filed Aug. 12, 2010. The disclosure of U.S. Provisional Patent Application 61/373,075 is hereby incorporated by reference in its entirety herein.

### FIELD

Embodiments of the present invention relate to metabolite biomarkers to diagnose, monitor, prognose and treat colorectal cancer, including methods, kits and systems using such metabolite biomarkers.

### BACKGROUND

Colorectal cancer (CRC) is the third most common type of cancer and the fourth most frequent cause of cancer mortality in the world. Weitz et al., 2005, Lancet, 365:153-165. The American Cancer Society estimated that a total of 1,437,180 new cancer cases and 565,650 deaths from cancer, including 148,810 new CRC cases and 49,960 deaths from CRC, would occur in the United States in 2008. Jemal et al., 2008, CA Cancer J. Clin., 58:71-96. Recent decades have witnessed a rapid increase in CRC morbidity in fast developing countries like China, especially in major cities where significant lifestyle alterations have occurred. Sung et al., 2005, Lancet Oncol., 6:871-876. Early and accurate diagnosis of CRC is of central importance for five-year survival and for less complicated surgery. Labianca et al., 2004, Crit. Rev. Oncol. Hematol., 51:145-170. Although CRC is a highly treatable and often curable disease when localized to the bowel, the prognosis for late stage CRC (e.g., recurrent metastatic disease) remains poor and is often fatal. Jemal et al., 2008, CA Cancer J. Clin., 58:71-96. Detecting CRC at an early stage improves survival rates dramatically: five-year survival rate is 93% for stage I patients but only 8% for stage IV patients. Sung et al., 2005, Lancet Oncol., 6:871-876.

To date, colonoscopy is the most effective screening tool for accurate diagnosis of precancerous lesions and cancer morbidity in the colon and rectum (e.g., aberrant crypt foci, polyps, and tumors). Winawer et al., 1993, N. Engl. J. Med., 329:1977-1981. Due to the invasive and unpleasant nature of the colonoscopy procedure, certain tumor biomarkers, such as carcinoembryonic antigen (CEA) and fecal occult blood testing (FOBT), have been used clinically, but with relatively poor sensitivity and specificity. Fletcher, 1986, Ann. Intern. Med., 104:66-73; Kronborg et al., 1996, Lancet, 348:1467-1471; Mandel et al., 1993, N. Engl. J. Med., 328:1365-1371.

A fundamental reason for the relative lack of progress worldwide in treating CRC is that the biology of this malignant disease is not sufficiently understood. Experimental studies have focused largely on understanding the transcriptional regulation of cancer-associated gene expression (Cardoso et al., 2007, Biochim. Biophys. Acta, 1775:103-137), while less research has been devoted to determining how this perturbed post-transcriptional regulation leads to the abnormal expression of downstream proteins and metabolites in this complex disease. Therefore, more effort is needed to improve understanding of CRC biology in order to identify new molecular targets and improve current cancer treatment and prevention strategies.

Metabolomics (Fiehn, et al., 2000, Nat. Biotechnol., 18:1157-1161) or metabonomics (Goodacre et al., 2004, Trends Biotechnol., 22:245-252), which is the study of metabolite profiles in a biological system under a given set of conditions, has become an approach to understanding the basic principles of relating chemical patterns in molecular biology and systems biology. Mitchell et al., 2002, Biologist (London), 49:217-221. Metabolomics has the capability of simultaneous analysis of hundreds and thousands of variables, which is useful in the study of complex diseases. Sunil et al., 2007, Trends in Analytical Chemistry, 26:6, 626-636. The clinical application of metabonomics for cancer study holds great potential for prognostic or predictive interpretation of cancer status as well as patient stratification due to its high sensitivity and the ability of quantitatively measuring entire composition of metabolites in a given biological specimen. Tiziani et al., 2009, Neoplasia, 11:269-276; Kim et al., 2009, Mol. Cell. Proteomics, 8:558-570; Denkert et al., 2006, Cancer Res., 66:10795-10804.

To date, global metabolic profiling of human biofluids (e.g., urine and sera) has been used to visualize the distinctive metabolic profiles of patients with various diseases, including inflammatory bowel disease (Marchesi et al., 2007, J. Proteome Res., 6:546-551), and type 2 diabetes (van Doom et al., 2007, Br. J. Clin. Pharmacol., 63:562-572). However, these studies were only able to identify overall patterns of metabolites in samples, and did not identify specific differential metabolite biomarkers capable of being used for diagnosis of diseases such as colorectal cancer.

Currently, a lack of detailed information about the disease-associated metabonome limits the ability of cancer biologists to understand the roles of metabolic pathways associated with CRC and its treatment. Metabolic profiling of tissue specimens of CRC patients has revealed significant variations in the metabolites of tumor tissue and normal mucosa. Chan et al., 2009, J. Proteome Res., 8:352-361; Denkert, et al., 2008, Mol. Cancer, 7:72; Piotto et al., 2008, Metabolomics, 5(3): 292-301. However, use of tissue samples for metabolic profiling is impractical for early and non-invasive CRC disease diagnosis. These studies also did not identify specific differential metabolite biomarkers capable of being used for diagnosis of colorectal cancer.

### SUMMARY

Certain embodiments of the present invention provide methods of diagnosing colorectal cancer in a subject comprising: (a) obtaining a sample from the subject; (b) determining a metabolite profile for the subject's sample by measuring the amount of each of one or more metabolite biomarkers; (c) comparing the subject's metabolite profile to a healthy control metabolite profile for the same one or more metabolite biomarkers in each sample; and (d) identifying differences between the subject's metabolite profile and the healthy control metabolite profile; wherein an increase or decrease in the level of one or more metabolite biomarkers in the subject's metabolite profile as compared to the healthy control metabolite profile indicates the presence of colorectal cancer in the subject.

Other embodiments of the present invention provide methods of determining the prognosis of a subject having colorectal cancer comprising: (a) obtaining a sample from the subject after diagnosis of colorectal cancer; (b) obtaining a sample from the subject after the subject has been treated for colorectal cancer; (c) determining a metabolite profile for the subject's samples obtained in steps (a) and (b) by measuring the amount of each of one or more metabolite biomarkers; (d) comparing the subject's metabolite profile before treatment to the subject's metabolite profile after treatment; and (e) identifying differences between the subject's metabolite profile before and after treatment; wherein an increase or decrease in the level of one or more metabolite biomarkers in the subject's metabolite profile after surgical treatment for colorectal cancer indicates the prognosis of the subject.

Additional embodiments of the present invention provide methods of identifying mechanisms of colorectal cancer pathogenesis comprising: (a) obtaining samples from subjects diagnosed with colorectal cancer; (b) obtaining samples from healthy subjects; (c) determining the metabolite profile in each of the samples obtained in steps (a) and (b) by measuring the amount of one or more metabolite biomarkers in each sample; (d) comparing the metabolite profile of subjects with colorectal cancer to the metabolite profile of healthy subjects; (e) identifying one or more metabolite biomarkers that differentiate between subjects with colorectal cancer and healthy subjects, or between subjects with different stage colorectal cancer, wherein the one or more metabolite biomarkers are present in different amounts in healthy subject, subjects with colorectal cancer and/or subjects having different stages of colorectal cancer; (f) identifying one or more cellular processes involving the one or more metabolite biomarkers, wherein the cellular processes comprise potential mechanisms of colorectal cancer pathogenesis.

Embodiments of the present invention also provide kits comprising: (a) a plurality of reference metabolites, wherein the reference metabolites are known to be increased or decreased in subjects having colorectal cancer as compared to healthy subjects; and (b) at least one container configured to hold the plurality of reference metabolites

Also, embodiments of the present invention provide systems comprising: (a) one or more reference metabolites having differential levels in subjects based on colorectal cancer disease status; (b) a analytical container configured to accept the one or more reference metabolites and at least one sample from at least one subject, wherein the at least one sample comprises at least one metabolite the same as at least one reference metabolite; and (c) an analytical device configured to detect and/or measure the at least one reference metabolites and the at least one metabolite in the subject sample within the analytical container.

### BRIEF DESCRIPTION OF THE FIGURES

The present invention may be better understood by referring to the non-limiting figures included herein.

FIG. 1 illustrates schematic representation of an embodiment of the present invention wherein gas chromatography—time-of-flight mass spectrometry (GC-TOFMS) and ultra performance liquid chromatography—quadropole-time-of-flight mass spectrometry (UPLC-QTOFMS), both in the negative ion electrospray ionization (ESI) mode and in the positive ESI mode, are used to assess the serum of CRC patients (grey ) and healthy controls (black ▪) to identify metabolic pathways (i.e., glycolysis, arginine and proline synthesis, fatty acid metabolism and oleamide metabolism) useful in the diagnosis of CRC.

FIG. 2 illustrates schematic representation of an embodiment of the present invention wherein GC-MS was used to assess the urine of CRC patients (grey ▴) and healthy controls (black ▪), as well as CRC model and healthy rats, to identify metabolic pathways useful in the diagnosis of CRC (e.g., tryptophan metabolism, tricarboxylic acid (TCA) cycle (a.k.a. the citric acid cycle), gut microflora metabolism).

FIG. 3 illustrates results from gas chromatography-time-of-flight mass spectrometry (GC-TOFMS) analysis using serum samples in accordance with an embodiment of the present invention. Panel A shows typical total ion current (TIC) chromatograms of biological samples obtained from a CRC patient and a healthy control (the key can be found in Table 2). Panel B shows an orthogonal projections to latent structures discriminant analysis (OPLS-DA) score plot discriminating the serum from CRC patients (grey ▴) and healthy controls (black ▪) using GC-TOFMS analysis.

FIG. 4 illustrates PCA score plots of CRC patients () and healthy controls (▪) using serum samples in accordance with an embodiment of the present invention. Panel A shows data generated from GC-TOFMS, with 6 components (R²Xcum=0.448, Q²cum=0.275). Panel B shows data generated from UPLC-QTOFMS (-mode), with 7 components (R²Xcum=0.488, Q²cum=0.401). Panel C shows data generated from UPLC-QTOFMS (+mode), with 6 components (R²Xcum=0.504, Q²cum=0.35).

FIG. 5 illustrates loading plots of OPLS-DA models using serum samples in accordance with an embodiment of the present invention. Data were generated from GC-TOFMS (Panel A), ultraperformance liquid chromatography-quadropole time-of-flight mass spectrometry (UPLC-QTOFMS) (-mode) (Panel B), and UPLC-QTOFMS (+mode) (Panel C). Each data point in a plot represents a single metabolite, while all the data points together represent the total population of metabolites in serum samples. The loading plots are a schematic representation of the mathematical distribution of metabolites in a sample reflecting the informativeness of each metabolite with regards to differentiating CRC patients from healthy controls. Metabolites towards the center of the plots are less informative, and metabolites towards the periphery of the plots are more informative and were focused on as potential metabolite biomarkers.

FIG. 6 illustrates how the levels of certain metabolites in serum changed in a consistent trend from stage I to IV of CRC (values normalized to stage I) in accordance with an embodiment of the present invention. Panel A shows the levels of metabolites 2-piperidinecarboxylic acid (▪), oleic acid (), lithocholic acid (♦) and myristic acid (▴). Panel B shows the levels of 1H-indole-3-acetic acid (♦), glycocholate (▪), and 3-hydrozybutanoic acid (♦).

FIG. 7 illustrates results from UPLC-QTOFMS analysis of patient serum samples in accordance with an embodiment of the present invention. Panel A shows OPLS-DDA scores plot discriminating the serum from CRC patients () and healthy controls (▪) using UPCL-QTOFMS negative ion mode analysis. Panel B shows OPLS-DA scores plot discriminating the serum from CRC patients and healthy controls using UPLC-QTOFMS positive ion mode analysis.

FIG. 8 illustrates a simplified metabolism pathway linking urea cycle to proline metabolism with glutamate acting as an important bridge according to an embodiment of the present invention. The (↓) symbol indicates down-regulation in the CRC group compared with the control group.

FIG. 9 illustrates typical total ion current (TIC) chromatograms and PCA score plot of human samples in accordance with an embodiment of the present invention. Panel A shoes TIC chromatograms from a preoperative CRC patient (upper panel) and a healthy control (lower panel), and Panel B shows a PCA score plot from preoperative CRC patients (▴) and healthy controls (▪).

FIG. 10 illustrates OPLS-DA scores plot and box plots of four typical differential metabolites from preoperative CRC patients and healthy controls in accordance with an embodiment of the present invention. Panel A shows an OPLS-DA model generated from preoperative CEC patients (▴) and healthy controls (▪). Panels B-E show box plots of typical identified differential metabolites from different metabolic pathways: succinate (Panel B), 5-hydroxyindoleacetate (Panel C), glutamate (Panel D), and 2-hydroxyhippurate (Panel E).

FIG. 11 illustrates a validation model of a 999 random permutation test for preoperative CRC patients and healthy controls in accordance with an embodiment of the present invention. Y-axis intercepts: R2=(0.0, 0.302), Q2=(0.0, −0.334).

FIG. 12 illustrates PLS and OPLS-DA of urinary metabolite profiles of the controls, CRC preoperative and CRC postoperative patients, and box plots of four differential metabolites in accordance with an embodiment of the present invention. Panel A shows PLS scores plot of control (▪), CRC preoperative (▴), and postoperative (). Panel B shows OPLS-DA scores plot of preoperative CRC patients (▴) and postoperative CRC patients (). Panels C—F show box plots of four differential metabolites: succinate (Panel C), 5-hydroxytryptophan (Panel D), phenylactylglutamate (Panel E), 2-hydroxyhippurate (Panel F).

FIG. 13 illustrates a line plot of six metabolites with characteristic expression levels among different CRC pathological stages in accordance with an embodiment of the present invention. The metabolites are: indoleacetate (♦), 2-methylpropanoate (x), P-hydroxyphenylacetate (▪), glutamate (*), 5-hydroxyindoleacetate (▴), and leucine (). Indoleacetate had much higher intensity in stage I patients; p-hydroxyphenylacetate had much higher intensity in stage II patients; 5-hydroxyindoleacetate had much higher intensity in stage III patients; 2-methylpropanoate had much lower intensity in stage IV patients; glutamate had a gradual increase from stage I to stage IV patients; leucine had much higher level in stage I patients and a gradual decrease from stage I to stage IV patients. Data were normalized to the average of each metabolite.

FIG. 14 illustrates a validation model of a 999 random permutation test for preoperative CRC patients (▴—R2) and postoperative CRC patients (▪—Q2) in accordance with an embodiment of the present invention. Y-axis intercepts: R2=(0.0, 0.229), Q2=(0.0, −0.229).

FIG. 15 illustrates OPLS-DA scores plot of urinary metabolite profiles of DMH-induced precancerous lesion rats and healthy rat controls, and box plots of four important differential metabolites in accordance with an embodiment of the present inventions. Panel A shows an OPLS-DA model generated from DMH-induced precancerous lesion rats (▴) and healthy rat controls (▪). Panels B-E show box plots of key identified differential metabolites: succinate (Panel B), 5-hydroxyindoleacetate (Panel C), spermidine (Panel D), and p-hydroxyphenylacetate (Panel E).

FIG. 16 illustrates typical aberrant crypt foci (ACF) lesions located in DMH-induced rat colon in accordance with an embodiment of the present invention. The lesions are marked by white arrows. (A: 3ACs/Foci; B: 2ACs/Foci).

FIG. 17 illustrates chromatograms and a PCA scores plot of rat urine in accordance with an embodiment of the present invention. Panel A shows a typical total ion current (TIC) chromatograms from a 1,2-dimethylhydrazine (DMH)-treated Sprague-Dawley CRC model rat (upper) and a healthy control (lower). Panel B shows a PCA scores plot between CRC model rat (▴) and control groups (▪).

FIG. 18 illustrates a validation model of a 999 random permutation test for 1,2-dimethylhydrazine (DMH)-treated Sprague-Dawley CRC model rats (▴—R2) and control rats (▪—Q2) in accordance with an embodiment of the present invention. Y-axis intercepts: R2=(0.0, 0.418), Q2=(0.0, −0.276).

FIG. 19 illustrates disturbed metabolic pathways associated with CRC morbidity in accordance with an embodiment of the present invention. Panel A shows the TCA cycle. Panel B shows tryptophan metabolism. Panel C shows the gut flora related metabolism. Panel D shows polyamine metabolism. The (+) symbol indicates higher levels in the 1,2-dimethylhydrazine (DMH)-treated Sprague-Dawley CRC model rats compared to control rats. The (−) symbol indicates lower levels in the CRC model rats compared to control rats. The (↑) symbol indicates a higher level in CRC patients compared to healthy controls; the (↓) symbol indicates a lower level in CRC patients compared to healthy controls.

FIG. 20 illustrates exemplary GC-TOFMS total ion current (TIC) chromatograms of serum samples from one CRC patient (top) and one healthy control (bottom) in accordance with an embodiment of the present invention.

FIG. 21 illustrates a PCA scores plot (Panel A) and an OPLS-DA (Panel B) scores plot for CRC patients (∘) and one healthy control (▪) in accordance with an embodiment of the present invention. Each plot point represents the data generated from a sample from a single individual.

FIG. 22 illustrates box-plots of representative differential metabolites between CRC patients and healthy controls urine in accordance with an embodiment of the present invention. The metabolites are 2-hydroxybutyric acid (Panel A), 3-hydroxybutyric acid (Panel B), Malic acid (Panel C), 4-hydroxy-proline (Panel D), Tryptophan (Panel E) and Oleic acid (Panel F).

FIG. 23 illustrates line plots of metabolites in serum with characteristic expression levels among different CRC pathological stages (X axis) relative to levels in healthy subjects in accordance with an embodiment of the present invention. The metabolites in the top panel are 2-oxo-butanoic acid (♦), 3-hydroxybutyric acid (▪), succinic acid (upper ▴ line), fumarate (x), tryptophan () and hexanoic acid (lower ▴ line). The metabolites in the bottom panel are citrate (♦), oxaloacetic acid (▪), palmitic acid (▴), oleic acid (x) and 9-octadecenoate ().

FIG. 24 illustrates typical GC-TOFMS total ion current (TIC) chromatograms of urine samples from one CRC patient (top) and one healthy control (bottom) in accordance with an embodiment of the present invention.

FIG. 25-PCA (Panel A) and OPLS-DA (Panel B) scores plot CRC patients and one healthy control. Each square represents one sample from healthy control, and each circle represents one sample from healthy control.

FIG. 26—Box-plots of typical differential metabolites between CRC patients and healthy controls. Panel A, p-cresol; Panel B, 5-oxo-proline; Panel C, N-acetyl-aspartic acid; Panel D, citric acid; Panel E, Hippurate; Panel F, 2-aminobutyric acid.

FIG. 27 illustrates line plots of metabolites in urine with characteristic expression levels among different CRC pathological stages (X axis) relative to levels in healthy subjects in accordance with an embodiment of the present invention. The metabolites are pyruvic acid (♦), 2-oxo-3-methyl-butanoic acid (▪), 2-aminobutyric acid (▴), fumarate (x), allantoin ( [symbol] ), methylcysteine (), lactate ( [symbol] ), tryptophan (darker —) and leucine (lighter —).

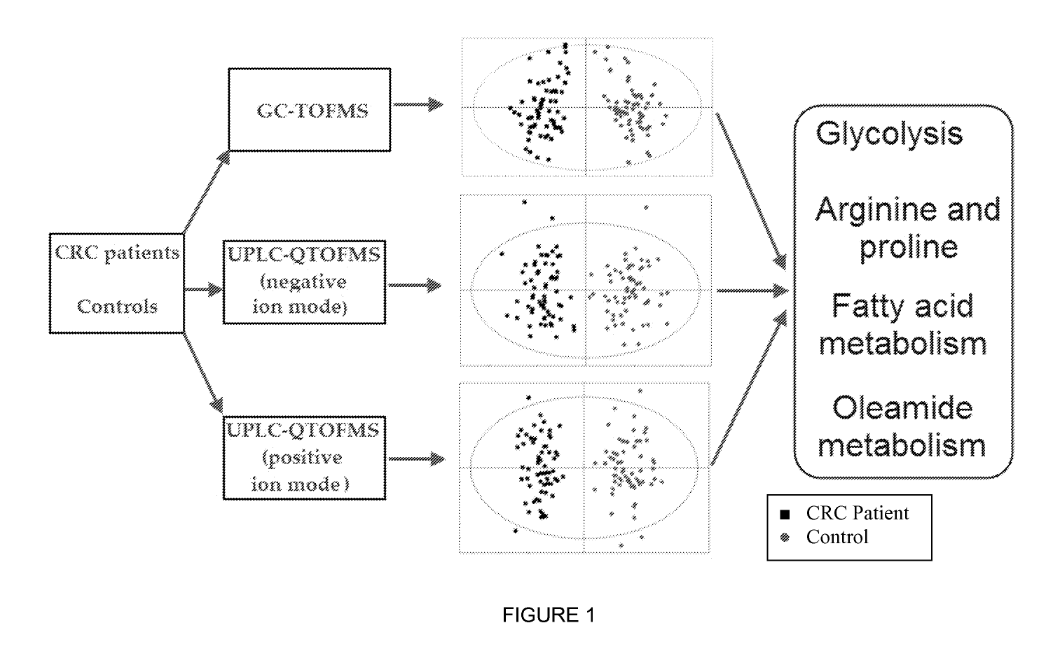

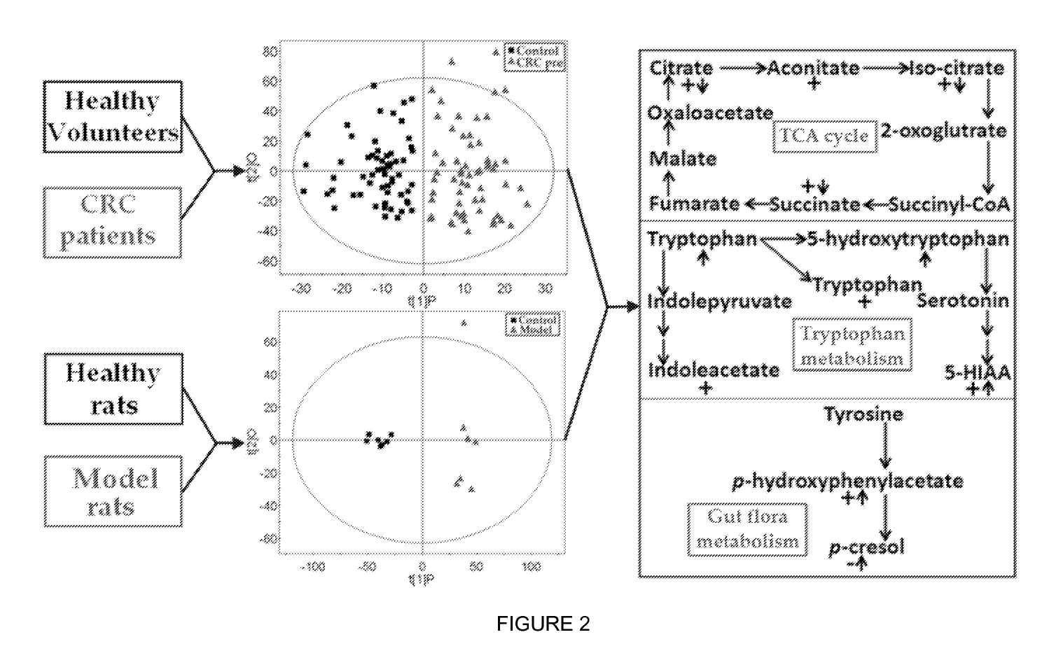

### DETAILED DESCRIPTION

Unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Also, where ranges are provided, it is understood that other embodiments within the specified ranges are to be included.

The following descriptions are meant to clarify, but not limit, the terms defined. If a particular term used herein is not specifically defined, such term should not be considered indefinite. Rather, terms are used within their ordinary meanings.

As used herein, a “subject” or an “individual” may be an animal. For example, the subject or individual may be a mammal. Also, the subject or individual may be a human. The subject or individual may be either a male or a female. The subject or individual may also be a patient, where a patient is an individual who is under dental or medical care and/or actively seeking medical care for a disorder or disease.

As used herein, “healthy” refers to an individual not having colorectal cancer or other related disorders.

As used herein, “metabolism” refers to the set of chemical reaction that occur in a living organism to maintain life. Metabolism is usually divided into two categories: catabolism and anabolism. Catabolism is a set of chemical reactions that breaks down organic matter (e.g., to harvest energy in cellular respiration). Anabolism is a set of chemical reactions that use energy to construct components of cells (e.g., protein and nucleic acid synthesis).

As used herein, a “metabolite” is an intermediate or product of metabolism. The term metabolite is generally restricted to small molecules. A “primary metabolite” is a metabolite directly involved in normal growth, development, and reproduction (e.g., alcohol). A “secondary metabolite” is a metabolite not directly involved in those processes, but that usually has an important ecological function (e.g., antibiotics, pigments). Some antibiotics use primary metabolites as precursors, such as actinomycin which is created from the primary metabolite, tryptophan. For the purposes of the present invention, the term metabolite does not refer to molecules such as nucleic acids or proteins. Rather, for the purposes of the present invention, the term metabolite refers to the small molecules (<1000 dalton) intermediates and products involved in metabolic pathways such as glycolysis, the citric acid (TCA) cycle, amino acid synthesis and fatty acid metabolism, amongst others.

As used herein, “metabolomics or “metabonomics” refers to the systematic study of metabolite profiles generated by biological processes in a biological system under a given set of conditions. “Metabolome” refers to refers to the complete set of small-molecule metabolites (such as metabolic intermediates, hormones and other signaling molecules, and secondary metabolites) to be found within a biological sample (e.g., a biological cell, tissue, organ or organism) that are the end products of cellular processes.

As used herein, “metabolite profile” or “metabolite biomarker profile” refers to a panel of metabolites that have been determined to have different levels (e.g., increased or decreased) in healthy subjects as compared to unhealthy subjects (e.g., subjects having colorectal cancer) or in different disease states (e.g., different stages of disease).

Thus, embodiments of the present invention use metabolomics analysis in diagnosis, determination of disease status, prognosis and treatment of human colorectal cancer.

Overexpression of many immediate-early response genes associated with growth and inflammation (e.g., proto-oncogenes, inflammatory mediators, and angiogenic growth factors) is commonly observed in CRC cells. These genetic modifications associated with colorectal carcinogenesis generally allow transformed cells to escape apoptosis while promoting proliferation, angiogenesis, and metastasis. Mendelsohn et al., 2001, In The Molecular Basis of Cancer, pp. 289-312. According to aspects of the invention, these changes can lead to significant alterations in downstream biochemical substances such as proteins and small-molecule metabolites. Small-molecule metabolites are the products of systemic biochemical regulations, and, in aspects of the invention, their levels can be regarded as the response of biological systems to genetic and environmental changes. See, e.g., Nicholson et al., 1999, Xenobiotica, 29:1181-1189.

According to aspects of the invention, metabonomics/metabolomics technology may use multivariate statistical techniques to analyze the highly complex data sets generated by high-throughput spectroscopy, such as nuclear magnetic resonance (NMR) and mass spectrometry (MS). See, e.g., Nicholson et al., 1999, Xenobiotica, 29:1181-1189; Williams et al., 2006, Mol. BioSyst., 2:174-183; Qiu et al., 2007, Anal. Chem. Acta, 583:277-283. In some aspects of the invention, the combined use of different types of spectroscopic platforms, such as GC-MS and LC-MS, can take advantage of complementary analytical outcomes and therefore, provide a broadened metabolic “window” for explaining the biological variations associated with pathophysiological conditions. In certain aspects of the invention, identifying metabolites that account for the differences between the metabolic profiles of people with CRC and healthy counterparts can reveal important underlying molecular mechanisms of the disease.

Certain embodiments of the present invention provide methods of diagnosing, monitoring, prognosing and treating colorectal cancer

In an embodiment of the present invention, a profiling method may be used to obtain data about metabolites in a patient sample. In some embodiments, the patient sample may be a bodily fluid. For example, the patient sample may be blood, serum, or urine. Other bodily fluids are also contemplated as patient samples according to various embodiments of the present invention.

In some embodiments, profiling methods according to the present invention may include gas chromatography and mass spectrometry. For example, the profiling methods according to an embodiment of the present invention may include gas chromatography-time-of-flight mass spectrometry (GC-TOFMS) and ultra-performance liquid chromatography-quadrupole time-of-flight mass spectrometry (UPLC-QTOFMS). In certain embodiments, more than one profiling method may be used to obtain data about metabolites in a patient sample. In some embodiments, one or more profiling methods may be used together with multivariate statistical techniques to assess a profile of metabolites in a patient sample.

In some embodiments of the present invention, the metabolites in a patient sample are assessed determine whether disease conditions are reflected in levels of individual metabolites in the metabolite profile (i.e., selected metabolites in the metabolome). In certain embodiments, identifying variations in the metablome that correspond to disease state may be used as non-invasive means for disease detection. In other embodiments of the present invention, knowledge of important metabolic variations associated with disease morbidity may be utilized for improved disease detection, diagnosis, and therapeutic strategies.

In some embodiments of the present invention, the disease may be cancer. In one embodiment of the present invention, the cancer may be colorectal cancer. In some embodiments of the present invention, the patient sample assessed for determination of metabolite profile is a bodily fluid, such as, for example, blood, serum or urine.

In an embodiment of the present invention, the metabolite profile in a serum sample from a patient with colorectal cancer may be assessed using profiling methods to determine whether the levels of certain metabolites are altered compared to a healthy individual, wherein variation between the metabolite profile of an individual with colorectal cancer may have abnormal levels of metabolites associated with glycolysis, arginine and/or proline metabolism, fatty acid metabolism and oleamide metabolism associated. In some embodiments of the present invention, the profiling methods may include gas chromatography-time-of-flight mass spectrometry (GC-TOFMS) and ultra-performance liquid chromatography-quadrupole time-of-flight mass spectrometry (UPLC-QTOFMS).

In another embodiment of the present invention, the metabolite profile in a urine sample from a patient with colorectal cancer may be assessed using profiling methods to determine whether the levels of certain metabolites are altered compared to a healthy individual, wherein variation between the metabolite profile of an individual with colorectal cancer may have abnormal levels of metabolites associated with the citric acid (TCA) cycle, tryptophan metabolism and/or gut flora metabolism. In other embodiments, the metabolite profile in a urine sample from a patient with colorectal cancer may be assessed using profiling methods to determine whether the levels of certain metabolites after surgical treatment of the cancer are altered in the patient compared to the levels of the metabolites prior to surgical treatment, wherein variation between the metabolite profile of an individual with colorectal cancer may have abnormal levels of metabolites associated with the citric acid (TCA) cycle, tryptophan metabolism and/or gut flora metabolism.

In one embodiment, the present invention provides methods of diagnosing colorectal cancer in a subject comprising: (a) obtaining a sample from the subject; (b) determining a metabolite profile for the subject's sample by measuring the amount of each of one or more metabolite biomarkers; (c) comparing the subject's metabolite profile to a healthy control metabolite profile for the same one or more metabolite biomarkers in each sample; and (d) identifying differences between the subject's metabolite profile and the healthy control metabolite profile; wherein an increase or decrease in the level of one or more metabolite biomarkers in the subject's metabolite profile as compared to the healthy control metabolite profile indicates the presence of colorectal cancer in the subject.

In certain embodiments, the one or more metabolite biomarkers have been previously identified as having increased or decreased levels in subjects having colorectal cancer. In some embodiments, the healthy control metabolite profile comprises representative levels of the one or more metabolite biomarkers in healthy subjects. For example, in some embodiments, healthy subject may be age- and gender-matched subjects. In some embodiments, the healthy control metabolite biomarker profile comprises a metabolite profile for the subject obtained at a preceding time when the subject was known to be healthy.

In various embodiments, the one or more metabolite biomarkers comprises at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

In some embodiments, the sample from the subject is a bodily fluid. In certain embodiments, the sample from the subject comprises blood, saliva, serum or urine. Alternatively, the sample may be a solid sample from the subject (e.g., tissue).

In certain embodiments, determining the subject's metabolite profile comprises subjecting the subject's sample to spectral analysis. In some embodiments, the spectral analysis comprises at least one of gas chromatography or mass spectrometry. Alternatively, in other embodiments, the determining the subject's metabolite profile comprises subjecting the subject's sample to analytical tests specific for the one or more metabolites (e.g., enzymatic, chemical, colorimetric, fluorometric, etc.).

In some embodiments, the level of one or more metabolite biomarkers in the subject's metabolite profile indicates at least one appropriate method of treatment for the subject's colorectal cancer.

In another embodiment, the present invention provides methods of determining the prognosis of a subject having colorectal cancer comprising: (a) obtaining a sample from the subject after diagnosis of colorectal cancer; (b) obtaining a sample from the subject after the subject has been treated for colorectal cancer; (c) determining a metabolite profile for the subject's samples obtained in steps (a) and (b) by measuring the amount of each of one or more metabolite biomarkers; (d) comparing the subject's metabolite profile before treatment to the subject's metabolite profile after treatment; and (e) identifying differences between the subject's metabolite profile before and after treatment; wherein an increase or decrease in the level of one or more metabolite biomarkers in the subject's metabolite profile after surgical treatment for colorectal cancer indicates the prognosis of the subject.

In some embodiments, the treatment for colorectal cancer is surgical treatment. In other embodiments, the treatment for colorectal is chemotherapy or radiation therapy. In other embodiments, any of these treatments, or a combination thereof, may be used.

In certain embodiments, the one or more metabolite biomarkers have been previously identified as having increased or decreased levels in subjects having colorectal cancer or in subjects with different stage colorectal cancers. In some embodiments, the one or more metabolite biomarkers comprises at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

In some embodiments, the sample from the subject is a bodily fluid. In certain embodiments, the sample from the subject comprises blood, saliva, serum or urine. Alternatively, the sample may be a solid sample from the subject (e.g., tissue).

In certain embodiments, determining the subject's metabolite profile comprises subjecting the subject's sample to spectral analysis. In some embodiments, the spectral analysis comprises at least one of gas chromatography or mass spectrometry. Alternatively, in other embodiments, the determining the subject's metabolite profile comprises subjecting the subject's sample to analytical tests specific for the one or more metabolites (e.g., enzymatic, chemical, colorimetric, fluorometric, etc.).

In some embodiments, the subject's metabolite biomarker profile after treatment for colorectal cancer indicates decreased morbidity.

In another embodiment, the present invention provides methods of identifying mechanisms of colorectal cancer pathogenesis comprising: (a) obtaining samples from subjects diagnosed with colorectal cancer; (b) obtaining samples from healthy subjects; (c) determining the metabolite profile in each of the samples obtained in steps (a) and (b) by measuring the amount of one or more metabolite biomarkers in each sample; (d) comparing the metabolite profile of subjects with colorectal cancer to the metabolite profile of healthy subjects; (e) identifying one or more metabolite biomarkers that differentiate between subjects with colorectal cancer and healthy subjects, or between subjects with different stage colorectal cancer, wherein the one or more metabolite biomarkers are present in different amounts in healthy subject, subjects with colorectal cancer and/or subjects having different stages of colorectal cancer; (f) identifying one or more cellular processes involving the one or more metabolite biomarkers, wherein the cellular processes comprise potential mechanisms of colorectal cancer pathogenesis.

In some embodiments, the metabolite profile of healthy subjects comprises a healthy control metabolite profile comprising one or more metabolite biomarkers having representative levels of the metabolite biomarkers in healthy subjects. For example, in some embodiments, healthy subject may be age- and gender-matched subjects.

In certain embodiments, the differentiating metabolite biomarkers comprise at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

In some embodiments, the sample from the subject is a bodily fluid. In certain embodiments, the sample from the subject comprises blood, saliva, serum or urine. Alternatively, the sample may be a solid sample from the subject (e.g., tissue).

In certain embodiments, determining the subject's metabolite profile comprises subjecting the subject's sample to spectral analysis. In some embodiments, the spectral analysis comprises at least one of gas chromatography or mass spectrometry. Alternatively, in other embodiments, the determining the subject's metabolite profile comprises subjecting the subject's sample to analytical tests specific for the one or more metabolites (e.g., enzymatic, chemical, colorimetric, fluorometric, etc.).

In some embodiments, the level of one or more metabolite biomarkers in the subject's metabolite profile indicates at least one appropriate method of treatment for the subject's colorectal cancer. In some embodiments of the invention, the one or more cellular processes comprising potential mechanisms of colorectal cancer pathogenesis indicates a desired method of treatment for colorectal cancer. In certain embodiments, selection of treatment for colorectal cancer comprises determining a method of treatment that will affect the one or more cellular processes comprising potential mechanisms of colorectal cancer pathogenesis.

In another embodiment, the present invention provides kits comprising: (a) a plurality of reference metabolites, wherein the reference metabolites are known to be increased or decreased in subjects having colorectal cancer as compared to healthy subjects; and (b) at least one container configured to hold the plurality of reference metabolites

In some embodiments, the plurality of reference metabolites comprises at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

In some embodiments, each of the plurality of reference metabolites are in separate containers. In certain embodiments, one or more known amounts of each of the plurality of reference metabolites.

In some embodiments, the container comprises a multi-chambered container. In certain embodiments, the container is configured to accept at least one biological sample from a subject.

In some embodiments, the container is configured for spectral analysis of metabolites within the container. In certain embodiments, the spectral analysis comprises at least one of gas chromatography or mass spectrometry. Alternatively, in other embodiments, the container may be configured for subjecting the subject's sample to analytical tests specific for the one or more metabolites (e.g., enzymatic, chemical, colorimetric, fluorometric, etc.).

In another embodiment, the present invention provides systems comprising: (a) one or more reference metabolites having differential levels in subjects based on colorectal cancer disease status; (b) a analytical container configured to accept the one or more reference metabolites and at least one sample from at least one subject, wherein the at least one sample comprises at least one metabolite the same as at least one reference metabolite; and (c) an analytical device configured to detect and/or measure the at least one reference metabolites and the at least one metabolite in the subject sample within the analytical container.

In some embodiments, the colorectal cancer disease status comprises the subject not having colorectal cancer, the subject having colorectal cancer or the subject having a particular stage of colorectal cancer.

In certain embodiments, the one or more reference metabolites comprise at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

In some embodiments, the analytical device comprises a spectral analytical device. In certain embodiments, the analytical device comprises at least one of a mass spectrometer or a gas chromatographer. Alternatively, other analytical devices may be used as appropriate to detect the read out of other analytical tests (including, e.g., a light spectrometer, a fluorometer, etc.).

In the various embodiments of the present invention, the one or more metabolites may comprise one or more of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine and p-hydroxyphenylacetate. In some embodiments, the one or more metabolites may comprise two, three, four, five, six, seven, eight, nine, ten, 11, 12, 13, 14, 15, 16, 17, 18, 19, 20, 21, 22 or 23 of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine and p-hydroxyphenylacetate.

In various embodiments of the present invention, the one or metabolites are serum metabolites or urine metabolites.

For example, in some embodiments, the serum metabolites are one or more of pyruvic acid, 2-hydroxybutyric acid, p-cresol, 3-hydroxybutyric acid, 2-aminobutanoic acid, urea, isoleucine, succinic acid, fumarate, methylmaleic acid, malic acid, aspartic acid, 4-hydroxy-proline, 2-oxo-pentanedioic acid, phenylalanine, asparagine, hypoxanthine, histidine, palmitic acid, tryptophan, linolic acid, oleic acid, octadecanoic acid or oleamide. In some embodiments, the one or more metabolites may comprise two, three, four, five, six, seven, eight, nine, ten, 11, 12, 13, 14, 15, 16, 17, 18, 19, 20, 21, 22, 23 or 24 of pyruvic acid, 2-hydroxybutyric acid, p-cresol, 3-hydroxybutyric acid, 2-aminobutanoic acid, urea, isoleucine, succinic acid, fumarate, methylmaleic acid, malic acid, aspartic acid, 4-hydroxy-proline, 2-oxo-pentanedioic acid, phenylalanine, asparagine, hypoxanthine, histidine, palmitic acid, tryptophan, linolic acid, oleic acid, octadecanoic acid and oleamide.

Also, in some embodiments, the urine metabolites are one or more of pyruvic acid, phenol, p-cresol, 2-aminobutyric acid, uracil, fumarate, methylmaleic acid, 5-oxoproline, 2-hydroxyglutaric acid, 2-oxopentanedioic acid, N-acetyl-aspartic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid. In some embodiments, the one or more metabolites may comprise two, three, four, five, six, seven, eight, nine, ten, 11, 12, 13, 14, 15, 16, 17, 18, 19, 20, 21, 22, 23, 24 or 25 of pyruvic acid, phenol, p-cresol, 2-aminobutyric acid, uracil, fumarate, methylmaleic acid, 5-oxoproline, 2-hydroxyglutaric acid, 2-oxopentanedioic acid, N-acetyl-aspartic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

In some embodiments, the metabolites are one or more of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

As shown in Examples 1 and 3, in some embodiments of the invention, the metabolite profile of CRC patients may be evaluated using serum samples, wherein the metabolite profile of CRC patients is compared to the metabolite profile of healthy controls to identify metabolic pathways useful in the diagnosis of CRC. Various embodiments of the present invention may include the use of gas chromatography and/or mass spectrometry to determine the metabolite profile in the CRC patient sample and healthy controls. For example, in an embodiment, metabolite profiles may be determined using gas chromatography time-of-flight mass spectrometry (GC-TOFMS) and ultraperformance liquid chromatography-quadrupole time-of-flight mass spectrometry in the negative ion mode (UPLC-QTOFMS) and in the positive ion mode (UPLC-QTOFMS). In additional embodiments, statistical analysis of the metabolic profile data may be performed. In some embodiments, statistical analysis may comprise multivariate statistics. In some embodiments, multivariate statistical analysis may be used to identify patterns of metabolites in the metabolite profile to generate a model profile of metabolites corresponding to disease state. For example, in some embodiments, orthogonal partial least-squares discriminate analysis (OPLS-DA) modeling may be performed using the metabolite profiles to assist in identifying variations between the metabolite profile of CRC patients and healthy controls. In some embodiments, CRC patient metabolite profiles include one or more metabolites involved in glycolysis, arginine and proline synthesis, fatty acid metabolism and/or oleamide metabolism that are different from the levels of the metabolites in healthy controls, wherein the differences in the metabolite levels are indicative of colorectal cancer.

As shown in Examples 2 and 4, in other embodiments of the invention, the metabolite profile of CRC patients may be evaluated using urine samples, wherein the metabolite profile of CRC patients is compared to the metabolite profile of healthy controls to identify metabolic pathways useful in the diagnosis of CRC. As described above, various embodiments of the present invention may include the use of gas chromatography and/or mass spectrometry to determine the metabolite profile in the CRC patient sample and healthy controls. For example, in an embodiment, metabolite profiles may be determined using gas chromatography mass spectrometry (GC-MS). In additional embodiments, statistical analysis of the metabolic profile data may be performed. In some embodiments, statistical analysis may comprise multivariate statistics. In some embodiments, multivariate statistical analysis may be used to identify patterns of metabolites in the metabolite profile to generate a model profile of metabolites corresponding to disease state. For example, in some embodiments, OPLS-DA modeling may be performed using the metabolite profiles to assist in identifying variations between the metabolite profile of CRC patients and healthy controls. In some embodiments, CRC patient metabolite profiles include one or more metabolites involved in the citric acid (TCA) cycle, tryptophan metabolism and/or gut flora metabolism that are different from the levels of the metabolites in healthy controls, wherein the differences in the metabolite levels are indicative of colorectal cancer.

In various embodiments, similar analysis may be conducted using an animal model to identify differences between the metabolite profile of healthy and diseased individuals. For example, animal models may include rodent models. In some embodiments, the rodent model may comprise 1,2-dimethylhydrazine (DMH)-treated Sprague-Dawley CRC model rats.

### EXAMPLES

### Example 1

### Metabolite Markers for Colorectal Cancer Identified in Serum (Initial Study)

### Materials and Methods for Serum Analysis

Chemicals. HPLC grade methanol, acetonitrile, and formic acid were purchased from Merck Chemicals (Darmstadt, Germany). Chloroform, pyridine, and anhydrous sodium sulfate were analytical grade and purchased from China National Pharmaceutical Group Corporation (Shanghai, China). L-2-chlorophenylalanine was purchased from Intechem Tech. Co. Ltd. (Shanghai, China). BSTFA (1% TMCS), heptadecanoic acid, methoxyamine, leucine-enkephalin were purchased from Sigma-Aldrich (St. Louis, Mo., USA).

Clinical samples. The patients, ages 42 to 74 years and diagnosed with CRC (32 colon cancers and 32 rectal cancers), were categorized according to histopathological features and stages according to TNM classification of malignant tumors: stage I, 9 patients; stage II, 27 patients; stage III, 20 patients; stage IV, 8 patients. Patients enrolled in this research were not on any medication before sample collection. The clinical diagnosis and pathological reports of all the patients were obtained from the hospital. The healthy volunteers, ages 42 to 69 years, were selected by a routine physical examination and any subjects with inflammatory conditions or gastrointestinal tract disorders were excluded. Body mass index (BMI) and carcinoembryonic antigen (CEA) levels for each CRC patient were also assessed. Clinical information on participants is provided in Table 1. Venous blood was collected in the morning before breakfast from a total of 64 CRC patients and 65 healthy volunteers at Cancer Hospital, Shanghai Medical College, Fudan University (Shanghai, China). The protocol was approved by the Cancer Hospital Institutional Review Board and all participants gave informed consent before they were involved in the study.

TABLE 1
Demographic and Clinical Chemistry
Characteristics of Human Subjects

| | CRC patients | Healthy controls |
|---|---|---|
| n | 64 | 65 |
| Age (median, range) | 59, 42-74 | 55, 42-69 |
| Male/female ratio | 35/29 | 34/31 |
| BMI (median, range) | 22.2, 16.4-28.9 | 23.5, 17.8-27.4 |
| CEA (>5.0 ng/ml) | 26 | N/A |
| Stage I | 9 | / |
| Stage II | 27 | / |
| Stage III | 20 | / |
| Stage IV | 8 | / |
| Colon cancer | 32 | / |
| Rectal cancer | 32 | / |

GC-TOFMS spectral acquisition of serum samples and data pretreatment. Serum metabolites were analyzed with chemical derivatization following our previously published procedure with minor modifications. Bao et al., 2009, J. Proteome Res., 8:1623-1630; Li et al., 2008, J. Proteome Res., 7:4775-4783. A 100 μL aliquot of serum sample was spiked with two internal standard solutions (10 μl L-2-chlorophenylalanine in water, 0.3 mg/ml; 10 μL heptadecanoic acid in methanol, 1 mg/mL) and vortexed for 10 seconds. The mixed solution was extracted with 300 μL of methanol:chloroform (3:1) and vortexed for 30 seconds. After storing for 10 minutes at −20° C., the samples were centrifuged at 12,000 g for 10 minutes. An aliquot of the 300 μL supernatant was transferred to a glass sampling vial to vacuum dry at room temperature. The residue was derivatized using a two-step procedure. First, 80 μL methoxyamine (15 mg/mL in pyridine) was added to the vial and kept at 30° C. for 90 minutes, followed by 80 μL BSTFA (1% TMCS) at 70° C. for 60 minutes.

Each 1 μL aliquot of the derivatized solution was injected in splitless mode into an Agilent 6890N gas chromatograph coupled with a Pegasus HT time-of-flight mass spectrometer (Leco Corporation, St. Joseph, USA). The CRC and control samples were run in the order of “control-CRC-control”, alternately, to minimize systematic analytical deviations. Separation was achieved on a DB-5 ms capillary column (30 m×250 μm I.D., 0.25 μm film thickness; (5%-phenyl)-methylpolysiloxane bonded and cross-linked; Agilent J&W Scientific, Folsom, Calif., USA), with helium as the carrier gas at a constant flow rate of 1.0 ml/min. The temperature of injection, transfer interface, and ion source was set to 270° C., 260° C., and 200° C., respectively. The GC temperature programming was set to 2 min isothermal heating at 80° C., followed by 10° C./min oven temperature ramps to 180° C., 5° C./min to 240° C., and 25° C./min to 290° C., and a final 9 min maintenance at 290° C. Electron impact ionization (70 eV) at full scan mode (m/z 30-600) was used, with an acquisition rate of 20 spectra/second in the TOFMS setting.

GC-TOFMS Data Analysis. The acquired MS files from GC/TOFMS analysis were exported in NetCDF format by ChromaTOF software (v3.30, Leco Co., CA, USA). CDF files were extracted using custom scripts (revised Matlab toolbox hierarchical multivariate curve resolution (H-MCR)) developed in the MATLAB 7.0 (The MathWorks, Inc, USA) for data pretreatment procedures such as baseline correction, de-noising, smoothing, alignment, time-window splitting, and multivariate curve resolution (based on multivariate curve resolution algorithm). Jonsson et al., 2004, Anal. Chem., 76:1738-1745; Jonsson et al., 2005, Anal. Chem., 77:5635-5642. The resulting three dimensional data set includes sample information, peak retention time and peak intensities. Internal standards and any known artificial peaks, such as peaks caused by noise, column bleed and BSTFA derivatization procedure, were removed from the data set. The resulting data was mean centered and unit variance scaled during chemometric data analysis in the SIMCA-p 12.0 Software package (Umetrics, Umeå, Sweden). Principal component analysis (PCA) and orthogonal projection to latent structures-discriminant analysis (OPLS-DA) were carried out to discriminate between CRC patients and healthy controls. In this study, the default 7-round cross-validation in SIMCA-p software package was applied with 1/7^{th} of the samples being excluded from the mathematical model in each round, in order to guard against over-fitting. Based on a variable importance in the projection (VIP) threshold of 1 from the 7-fold cross-validated OPLS-DA model, a number of metabolites responsible for the difference in the metabolic profiles of diseased individuals and healthy controls could be obtained. In parallel, the metabolites identified by the OPLS-DA model were validated at a univariate level using the nonparametric Wilcoxon-Mann-Whitney test from the Matlab statistical toolbox with the critical p-value set to 0.05. The corresponding fold change shows how these selected differential metabolites varied between the CRC and healthy control groups. Additionally, compound identification was performed by comparing the mass fragments with NIST 05 Standard mass spectral databases in NIST MS search 2.0 (NIST, Gaithersburg, Md.) software with a similarity of more than 70% and finally verified by available reference compounds.

UPLC-QTOFMS spectral acquisition of serum samples and data pretreatment. Each 100 μL serum was used for metabolite extraction prior to UPLC-QTOFMS analysis. The metabolite extraction procedure was carried out after adding 100 μL of water (containing 0.1 mg/mL L-2-chlorophenylalanine as the internal standard) and 400 μL of a mixture of methanol and acetonitrile (5:3) in the serum. After vortexing for 2 min, the mixture was kept at room temperature for 10 min, then centrifuged at 14,500 g for 20 min. The supernatant was filtered through a syringe filter (0.22 μm) and placed into the sampling vial pending UPLC-QTOFMS analysis.

A 5 μL aliquot of the filtrate was injected into a 10 cm×2.1 mm, 1.7 μm BEH C18 column (Waters, USA) held at 40° C. using an Acquity ultra performance liquid chromatography system (Waters, USA). Control and CRC samples were alternately run. The column was eluted with a linear gradient of 1-20% B over 0-1 min, 20-70% B over 1-3 min, 70-85% B over 3-8 min, 85-100% B over 8-9 min, the composition was held at 100% B for 0.5 min. For positive ion mode (ESI+) where A=water with 0.1 formic acid and B=acetonitrile with 0.1% formic acid, while A=water and B=acetonitrile for negative ion mode (ESI−). The flow rate was 0.4 mL/min. All the samples were kept at 4° C. during the analysis.

The mass spectrometric data was collected using a Waters Q-TOF micro MS (Manchester, UK) equipped with an electrospray ionization source operating in either positive or negative ion mode. The source temperature was set at 120° C. with a cone gas flow of 50 L/h, a desolvation gas temperature of 300° C. with a desolvation gas flow of 600 L/h. In the case of positive and negative ion modes the capillary voltage was set to 3.2 kV and 3 kV, and the cone voltage of 35 V and 50 V, respectively. Centroid data was collected from 50 to 1000 m/z with a scan time of 0.3 sec and interscan delay of 0.02 sec over a 9.5 min analysis time. Waters MassLynx™ Software (Waters Corp.) was used for system controlling and data acquisition. Leucine enkephalin was used as the lock mass (m/z 556.2771 in ESI+ and 554.2615 in ESI−) at a concentration of 100 ng/mL and flow rate of 0.2 mL/min, with a lockspray frequency of 20 sec.

UPLC-QTOFMS Data Analysis. The UPLC-QTOFMS ES⁺ and ES⁻ raw data were analyzed by the MarkerLynx™ Applications Manager version 4.1 (Waters, Manchester, U.K.) using parameters reported in our previous work. Xie et al., 2008, J. Sep. Sci., 31:1015-1026. A list of the ion intensities of each peak detected was generated, using retention time (RT) and the m/z data pairs as the identifier for each ion. The resulting three-dimensional matrix contained arbitrarily assigned peak indexes (retention time-m/z pairs), sample names (observations), and ion intensity information (variables). To obtain consistent differential variables, the resulting matrix was further reduced by removing any peaks with missing value (ion intensity=0) in more than 40% of the samples from both CRC and healthy groups. The ion peaks generated by the internal standard were also removed. The data was then normalized by dividing the sum of all peak intensities within the sample. The resulting data set was imported to SIMCA-p software 12.0 (Umetrics, Umeå, Sweden) for multivariate statistical analysis similar to the GC-TOFMS data analysis using PCA and OPLS-DA. Compound identification was performed by comparing the accurate mass spectrum and retention time of reference compounds in our established library.

### Results

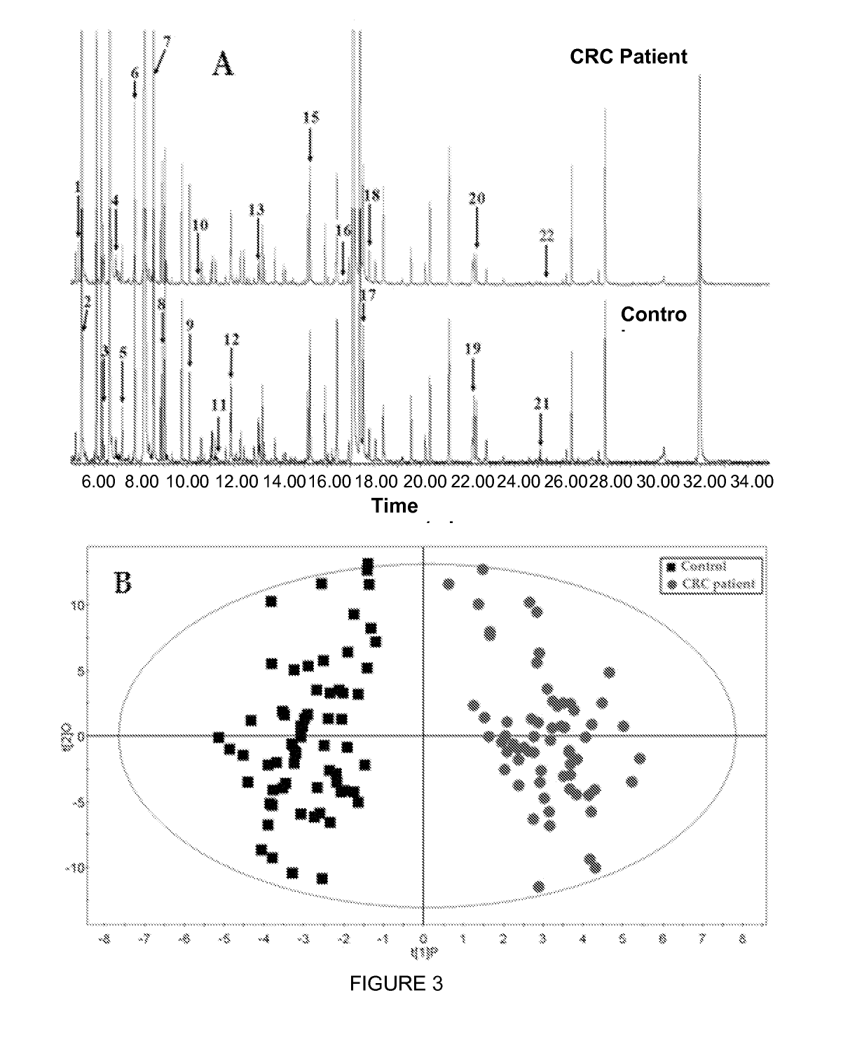

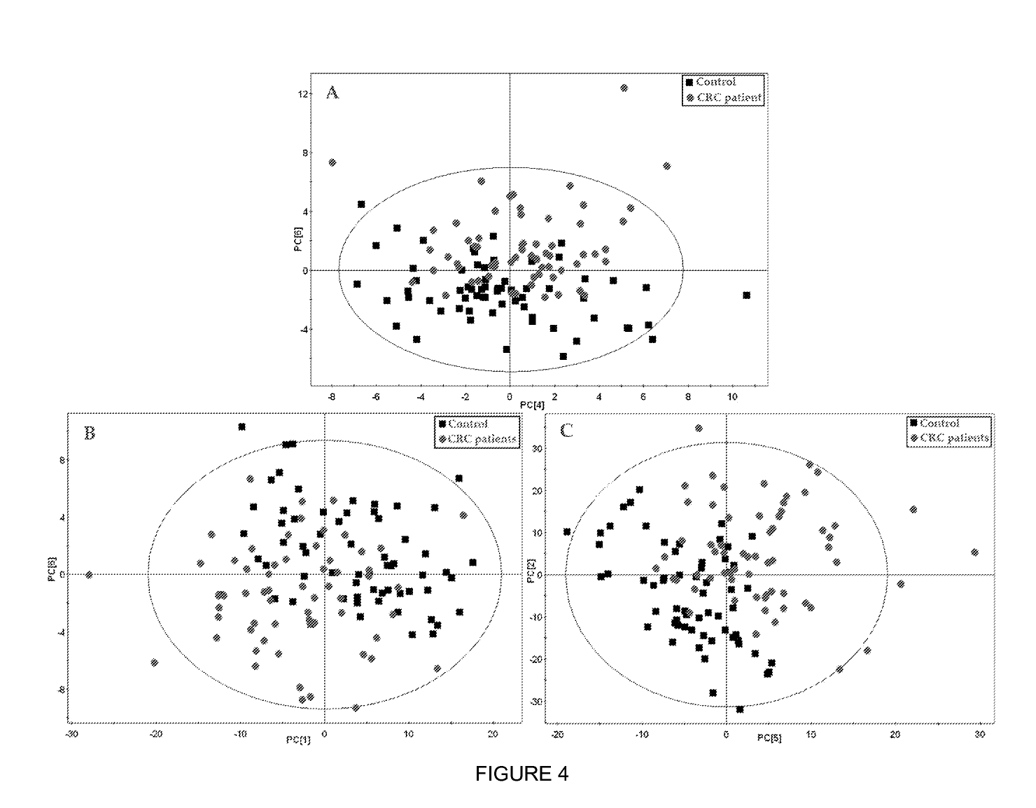

GC-TOFMS analysis. Typical GC-TOFMS total ion current (TIC) chromatograms of serum samples from a cancer patient and a healthy control are shown in FIG. 3A. The resulting spectrum was analyzed with H-MCR toolbox in Matlab and SIMCA-p software. After removing two internal standards, a total of 223 variables were used in the following analysis. In the PCA scores plot, we can see the separation trends between CRC patients and controls as shown in FIG. 4A. The OPLS-DA model demonstrated satisfactory modeling and predictive abilities using one predictive component and two orthogonal components (R²Ycum=0.901, Q²cum=0.758), achieving a distinct separation between the metabolite profiles of the two groups (FIG. 3B).

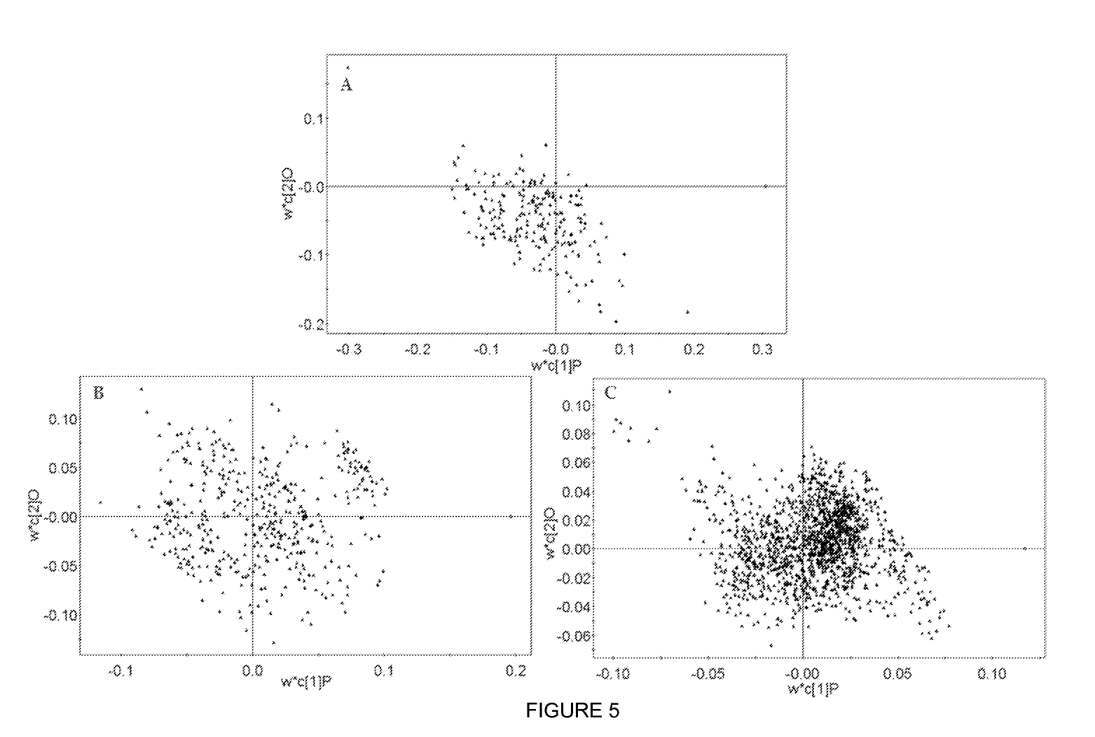

Notably, nine early stage CRC patients (stage I), in addition to the patients belonging to stage II-IV, were correctly discriminated from the healthy controls by the OPLS-DA model. The loading plots were provided in the supporting information (FIG. 5A). However, a separate OPLS-DA model without the healthy group failed to discriminate different pathological stages (I to IV) of CRC patients.

Twenty-two metabolites were identified using MS spectral databases and fourteen were confirmed using reference standards among the differential variables using VIP values (VIP>1) in the OPLS-DA model and the Wilcoxon-Mann-Whitney test (p<0.05) (Table 2). Among the identified metabolites, oleamide was the serum metabolite found to be most depleted in the CRC patients, compared to controls, showing the greatest fold change (FC=−3). Pyruvate was the metabolite most increased (FC=2.1) in CRC patients. The most significantly altered serum metabolites included decreased lysine, tryptophan, citrulline and tyrosine, and elevated lactate, 2-hydroxybutrate, and 3-hydroxybutrate in the serum of CRC patients compared with healthy controls.

TABLE 2
Differential metabolites derived from OPLS-DA mode of
GC-TOFMS analysis with Wilcoxon-Mann-Whitney test

| | | Retention | | | | |
|---|---|---|---|---|---|---|
| | | Time | | | | Chemical |
| No. | Metabolite^{a} | (min) | VIP^{C} | P^{d} | FC^{e} | Class |
| 1 | Pyruvate^{b} | 5.343 | 2.9 | 9.57E−13 | 2.1 | Organic |
| | | | | | | acid |
| 2 | Lactate | 5.497 | 1.3 | 8.99E−03 | 1.3 | Organic |
| | | | | | | acid |
| 3 | 2-hydroxy- | 6.452 | 1.4 | 2.84E−04 | 1.4 | Organic |
| | butanoic | | | | | acid |
| | acid | | | | | |
| 4 | 3-hydroxy- | 6.956 | 1.1 | 0.017 | 1.4 | Organic |
| | butanoic | | | | | acid |
| | acid^{b} | | | | | |
| 5 | urea | 7.231 | 1.5 | 1.15E−03 | −1.4 | Amine |
| 6 | Valine^{b} | 7.762 | 1.6 | 3.09E−05 | −1.5 | Amino |
| | | | | | | acid |
| 7 | Leucine^{b} | 8.563 | 1.6 | 1.20E−04 | −1.5 | Amino |
| | | | | | | acid |
| 8 | Proline^{b} | 8.948 | 1.1 | 0.0101 | −1.3 | Amino |
| | | | | | | acid |
| 9 | Threonine^{b} | 10.11 | 1.4 | 0.0053 | −1.4 | Amino |
| | | | | | | acid |
| 10 | Threonic | 10.727 | 1.5 | 5.51E−05 | −1.6 | Organic |
| | acid | | | | | acid |
| 11 | Malic acid | 11.442 | 1.4 | 3.82E−03 | 1.3 | Organic |
| | | | | | | acid |
| 12 | 4-hydroxy- | 11.928 | 1.6 | 1.71E−04 | −1.5 | Amino |
| | proline^{b} | | | | | acid |
| 13 | Citrulline^{b} | 13.059 | 1.8 | 3.93E−04 | −1.4 | Amino |
| | | | | | | acid |
| 14 | 2- | 15.261 | 1.9 | 0.0079 | −1.3 | Organic |
| | Piperidine- | | | | | acid |
| | carboxylic | | | | | |
| | acid | | | | | |
| 15 | Ornithine^{b} | 16.028 | 1.6 | 3.93E−04 | −1.4 | Amino |
| | | | | | | acid |
| 16 | Hippurate | 16.32 | 1.3 | 0.0377 | −1.5 | Aromatic |
| | | | | | | compound |
| 17 | Lysine^{b} | 17.551 | 1.3 | 0.00291 | −1.4 | Amino |
| | | | | | | acid |
| 18 | Tyrosine^{b} | 17.807 | 1.6 | 9.89E−05 | −1.5 | Amino |
| | | | | | | acid |
| 19 | Tryptophan^{b} | 22.225 | 2 | 2.04E−05 | −1.6 | Amino |
| | | | | | | acid |
| 20 | Oleic acid | 22.495 | 1.1 | 0.0423 | 1.1 | Fatty acid |
| 21 | Oleamide^{b} | 25.135 | 4.5 | 2.04E−15 | −3 | Fatty acid |
| | | | | | | amine |
| 22 | Uridine | 25.392 | 1.1 | 1.14E−06 | −1.7 | Pyrimidine |

^{a}Metabolites are identified using available library databases;
^{b}verified by reference compounds.
^{c}Variable importance in the projection (VIP) was obtained from OPLS-DA with a threshold of 1.0.
^{d}P-value and fold change (FC) are calculated from nonparametric Wilcoxon-Mann-Whitney test.
^{e}FC with a positive value indicates a relatively higher concentration present in CRC patients while a negative value means a relatively lower concentration as compared to the healthy controls.

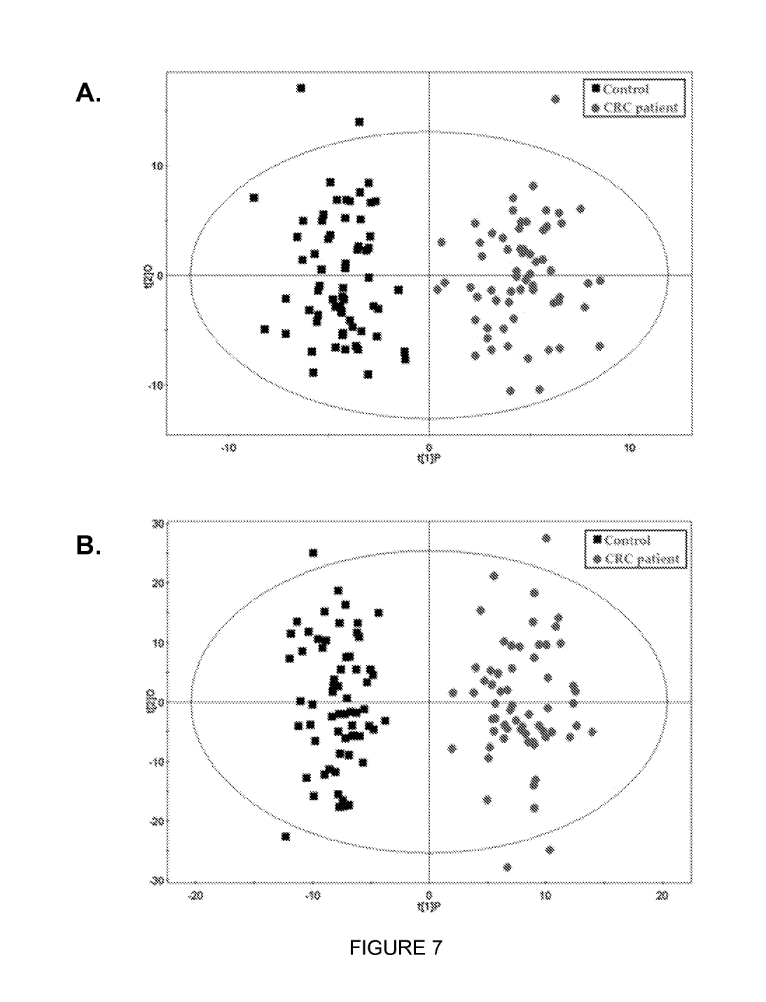

UPLC-QTOFMS analysis. Due to an insufficient volume of serum samples, one CRC patient and two healthy controls were not analyzed in the UPLC-QTOFMS platform. After removing those peaks with missing value in more than 40% of individuals in both groups, a total of 1570 peaks of ESI⁺ and 450 peaks of ESI⁻ were obtained from the acquired data using MarkerLynx™ software. These two sets of data were normalized and induced to SIMCA-P 12.0, respectively. PCA scores plots showed the separation trend between CRC patients and controls in negative ion mode (FIG. 4B) and positive ion mode (FIG. 4C). OPLS-DA model score plots from both negative (FIG. 7A) and positive (FIG. 7B) ion mode showed clear separations between CRC patients and healthy controls using one predictive component and three orthogonal components, with satisfactory modeling and predictive abilities (R²Ycum=0.881, Q²cum=0.633 for negative and R²Ycum=0.918, Q²cum=0.812 for positive mode, respectively). The loading plots are shown in FIG. 5B and FIG. 5C. Similar to GC-TOFMS analysis, the model failed to distinguish the stages of CRC.

A total of 16 metabolites identified from UPLC-QTOFMS positive ion mode and negative ion mode are shown in Table 3 using a method similar to GC-TOFMS analysis. Among these, five metabolites were also detected in the GC-TOFMS analysis with the same direction. Those metabolites include increased levels of pyruvate and lactate, and decreased levels of tryptophan, tyrosine, and uridine in the CRC patients compared to healthy controls. Table 4 lists seven metabolites identified as having consistent trends in alteration (increase or decrease) from stage I to IV of CRC. The values were normalized to the corresponding mean values from stage I patients.

TABLE 3
Differential metabolites derived from OPLS-DA mode of
UPLC-QTOFMS analysis with Wilcoxon-Mann-Whitney test

| | | | | | | |
|---|---|---|---|---|---|---|
| | | Reten- | | | | |
| | | tion | | | | |
| | | Time | | | | Chemical |
| No. | metabolite^{a} | (min) | VIP^{b} | P^{c} | FC^{d} | Class |

Negative ion mode

| | | | | | | |
|---|---|---|---|---|---|---|
| 1 | Glycerol | 0.683 | 1.7 | 3.89E−05 | 1.5 | Organic |
| | phosphate | | | | | acid |
| 2 | Pyruvic acid | 0.698 | 1.3 | 0.0001 | 1.5 | Organic |
| | | | | | | acid |
| 3 | Lactate | 0.718 | 1.3 | 0.0013 | 1.4 | Organic |
| | | | | | | acid |
| 4 | Tyrosine | 1.119 | 1.1 | 0.0098 | −1.3 | Amino |
| | | | | | | acid |
| 5 | Uridine | 1.287 | 1.1 | 0.0114 | −1.3 | Pyrimidine |
| 6 | Phenylalanine | 1.457 | 1.0 | 0.0334 | −1.2 | Amino |
| | | | | | | acid |
| 7 | Tryptophan | 1.653 | 1.0 | 0.0381 | −1.2 | Amino |
| | | | | | | acid |
| 8 | Myristic acid | 3.983 | 1.2 | 0.0034 | −1.3 | Fatty acid |
| 9 | Palmitic acid | 4.655 | 1.1 | 0.0009 | −1.4 | Fatty acid |
| 10 | Nervonic acid | 6.082 | 1.3 | 0.0032 | −1.3 | Fatty acid |

Positive ion mode

| | | | | | | |
|---|---|---|---|---|---|---|
| 11 | Arginine | 0.7031 | 1.2 | 0.0129 | −1.3 | Amino |
| | | | | | | acid |
| 12 | Carnitine | 0.7249 | 1.2 | 0.0073 | 1.3 | Carnitines |
| 13 | Glutamic acid | 0.7303 | 2.0 | 1.21E−06 | −1.7 | Amino |
| | | | | | | acid |
| 14 | Nicotinamide | 1.2926 | 1.0 | 0.0032 | −1.2 | Amino |
| | | | | | | acid |
| 15 | Dopamine | 1.3779 | 1.2 | 0.0098 | −1.3 | Aromatic |
| | | | | | | compound |

^{a}Metabolites are identified by comparing the exact mass and retention time of our reference metabolites in our laboratory.
^{b}Variable importance in the projection (VIP) was obtained from OPLS-DA with a threshold of 1.0.
^{c}P-value and fold change (FC) are calculated from nonparametric Wilcoxon-Mann-Whitney test.
^{d}FC with a positive value indicates a relatively higher concentration present in CRC patients while a negative value means a relatively lower concentration as compared to the healthy controls.

TABLE 4
Consistent pattern of metabolite alteration in different CRC stages.

| | Stage 1 | | Stage 2 | | Stage 3 | | Stage 4 | | Analytical |
|---|---|---|---|---|---|---|---|---|---|
| Metabolite | mean | SEM^{a} | mean | SEM | mean | SEM | mean | SEM | Platform |
| myristic acid | 1 | 0.39 | 0.79 | 0.34 | 0.62 | 0.45 | 0.57 | 0.35 | UPLC- |
| | | | | | | | | | QTOFMS |
| | | | | | | | | | (ESI−) |
| 2-piperidine- | 1 | 1.40 | 0.86 | 1.57 | 0.48 | 0.27 | 0.44 | 0.24 | GC- |
| carboxylic acid | | | | | | | | | TOFMS |
| 1H-Indole-3- | 1 | 1.08 | 0.77 | 0.74 | 0.73 | 0.41 | 0.42 | 0.17 | GC- |
| acetic acid | | | | | | | | | TOFMS |
| glycocholate | 1 | 0.89 | 0.85 | 0.76 | 0.72 | 0.84 | 0.57 | 0.84 | UPLC- |
| | | | | | | | | | QTOFMS |
| | | | | | | | | | (ESI+) |
| 3-hydroxy- | 1 | 1.69 | 1.67 | 2.08 | 2.05 | 4.76 | 2.24 | 3.07 | GC- |
| butanoic acid | | | | | | | | | TOFMS |
| oleic acid | 1 | 0.42 | 1.34 | 0.58 | 1.43 | 0.89 | 1.61 | 1.70 | GC- |
| | | | | | | | | | TOFMS |
| lithocholic acid | 1 | 0.51 | 1.42 | 1.33 | 2.12 | 4.36 | 2.65 | 2.61 | UPLC- |
| | | | | | | | | | QTOFMS |
| | | | | | | | | | (ESI+) |

^{a}Standard error of the mean.

### Discussion

As metabonomic data often contains a large number of variables that are interrelated, multivariate statistical methods such as PCA and OPLS-DA coupled with a univariate statistical method such as Wilcoxon-Mann-Whitney test were used to assess patient samples. Feature selection from variables was performed using two parameters, a threshold of 1 by VIP and a p-value set to 0.05, to identify variables with biological significance as endpoints of altered interdependent biochemical pathways.

GC-TOFMS based metabonomic study identified significant variations between CRC patients and healthy controls in 22 metabolites including oleamide, proline, citrulline, ornithine, and 3-hydroxybutrate. UPLC-QTOFMS was able to identify 16 differential metabolites including arginine, glutamate, palmitate, and carnitine. Among them 5 differential metabolites, pyruvate, lactate, tryptophan, tyrosine, and uridine were identified in both analytical platforms with the same alteration direction (up- or down-regulation). Thus, the combination of the two analytical platforms broadened the spectrum of detected metabonomes and cross-validated each other.

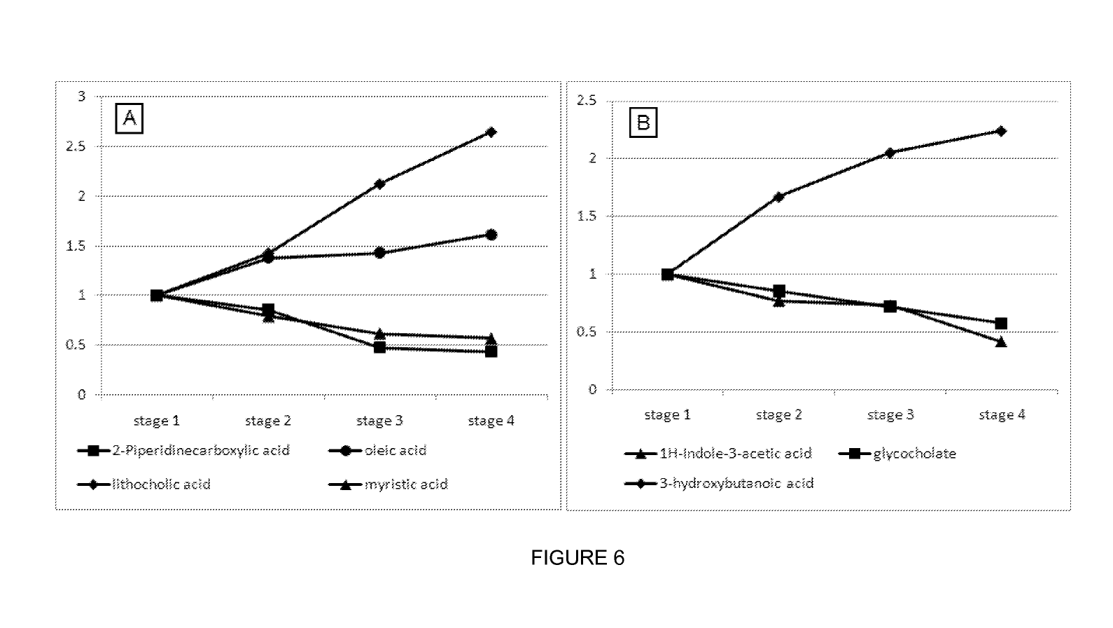

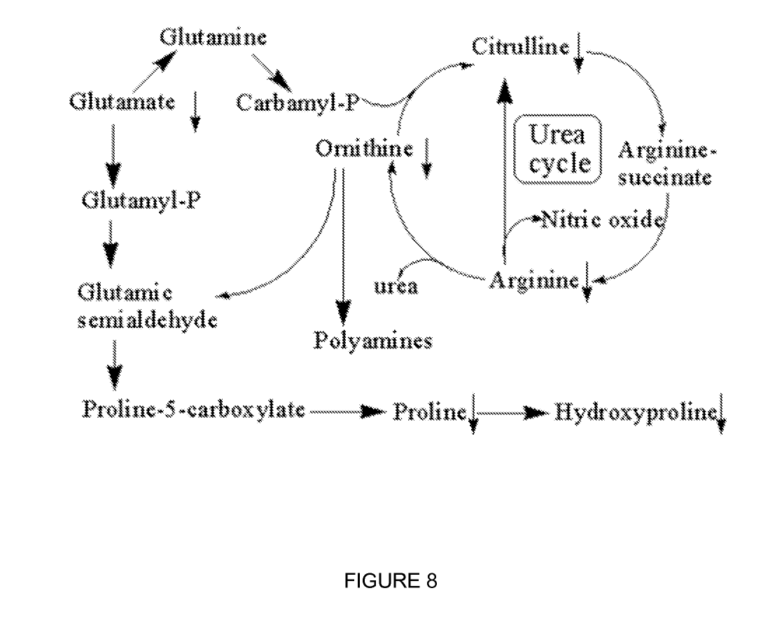

The OPLS-DA models derived from the described GC-TOFMS and UPLC-QTOFMS (both positive and negative ion mode) metabolic analysis showed good and similar separations between patients with CRC and healthy controls, highlighting the diagnostic potential of this non-invasive analytical approach. The histopathological diagnosis of CRC reflects a chronological development of impairment in the intestinal tract, as a relatively “static” readout of the disease, while variations in metabolites can be regarded as “real time” readout, reflecting the dynamic states of the CRC pathophysiology. There are a number of serum metabolites which showed a consistent trend of alteration (up- or down-regulation) from stage I to IV of CRC patients (Table 4; FIGS. 6 and 8).

To facilitate this metabolic approach into clinical use and to understand the underlying biological alterations associated with CRC morbidity, metabolite identification is important. Detection using both instruments may allow greater confidence to be placed on the measurement made for this subset of differential metabolites, including higher levels of pyruvate and lactate, and lower levels of tryptophan, tyrosine, and uridine in the CRC patients compared to the healthy controls. Pyruvate and lactate are the intermediate and the endpoint product of glycolysis. Increased glycolysis is associated with many tumors or cancer cells, even in the presence of oxygen, which is known as the Warburg effect. Gatenby and Gillies, 2004, Nat. Rev., 4:891-899. Recent proteomic analysis of colonic tissues from CRC patients has identified increased glycolysis as an important metabolic variation associated with CRC morbidity. Bi et al., 2006, Mol. Cell. Proteomics, 5:1119-1130. Thus, the abnormal accumulation of pyruvate and lactate in the CRC patients may be the result of a higher energy demand in the solid colorectal tumor tissues.

Many serum amino acids were found to be down-regulated in CRC patients compared with healthy controls (Tables 2 and 3). This observation may indicate an over utilization of amino acids in the tumor tissue, as evidenced by recent tissue based metabolic profile research that up-regulation of most amino acids was detected in CRC carcinoma tissue compared to normal tissue. Denkert et al., 2008, Mol. Cancer, 7:72. The complementary results between serum and CRC tissue are similar to previous reports of decreased histamine levels in the serum of colon cancer patients compared with healthy controls (Previati et al., 2002, J. Chromatogr., B: Anal. Technol. Biomed. Life Sci., 780:331-339), and an up-regulation in colorectal cancer tissue compared to normal tissue. Garcia-Cabellero et al., 1988, Agents Actions, 23:357-360.

The UPLC-QTOFMS positive ion mode analysis described herein showed a decrease in the level of arginine in CRC patients. L-arginine has been reported to inhibit chemical-induced colorectal cancer and can reduce cell proliferation in patients with colorectal adenoma. Ma et al., 1996, World J. Surg., 20:1087-1091; Ma et al., 2007, Clin. Cancer Res., 13:7407-7412. It has been suggested that the underlying correlation between arginine and colorectal cancer is its regulation of the immune system via nitric oxide. Ma et al., 2007, Clin. Cancer Res., 13:7407-7412; Tong and Barbul, 2004, Mini Rev. Med. Chem., 4:823-832. Arginine is an intermediate of the urea cycle as illustrated in FIG. 8. The disturbed arginine metabolism associated with CRC morbidity can be confirmed by our observations in GC-TOFMS analysis, where ornithine, citrulline and urea were found to be down-regulated in CRC patients compared to healthy controls. Moreover, arginine metabolism is connected with glutamate metabolism and proline metabolism via glutamic semialdehyde. Morris, 2007, J. Nutr., 137:1602 S-1609S. The observation of depleted glutamate, proline, and hydroxyproline in the serum of CRC patients may further support the down-regulation of arginine metabolism, and reflect disturbed proline metabolism in CRC patients as well. The abnormal proline level in CRC patient serum may also indicate disturbed expression of proline oxidase (catalyzes the first step of proline degradation to pyrroline-5-carboxylate), which was reported to highly correlate with p53-dependent inhibition of apoptosis in colorectal cancer. Liu et al., 2008, Oncogene, 27:6723-6737. Thus, depletion of arginine and proline metabolism may be an indication of an immune system impairment related to nitric oxide metabolism and may correlate with abnormal apoptosis in CRC patients.

An alternation of fatty acid metabolism was also observed. GC-TOFMS analysis detected higher levels of 3-hydroxybutanoic acid, the endpoint product of fatty acids β-oxidation, in the CRC patients, while UPLC-QTOFMS analysis revealed decreased levels of palmitic acid, myristic acid and carnitine, the carrier of fatty acid, in the CRC patients compared to healthy controls. These findings are an indication of the dysfunction of fatty acid β-oxidation metabolism, which can be confirmed by previous proteomic research. Mazzanti et al., 2006, Am. J. Physiol.: Gastrointest. Liver Physiol., 290:G1329-G1338.

Oleamide (or cis-9,10-octadecenoamide) was the most down-regulated metabolite observed in all of the differential metabolites obtained from GC-TOFMS analysis. It is a primary fatty acid amide reported to mediate conjugated linoleic acid inhibition of Caco-2 in colon cancer cell growth. Kim et al., 2002, Anticancer Res., 22:2193-2197. Oleamide has also been reported to enhance the activity of certain types of serotonin receptors (e.g., 5-HT_{1A}, 5-HT_{2A}, and 5-HT_{2c}). Boger, et al., 1998, Curr. Pharm. Des., 4:303-314; Thomas et al., 1997, Proc. Natl. Acad. Sci. U.S.A., 94:14115-14119. Down-regulation of tryptophan, the precursor of serotonin, in CRC patients may correlate with the alternated serotonin function. Oleamide can further influence CRC morbidity through competitively inhibiting the degradation of endocannabinoids via fatty acid amides hydrolase. Bisogno et al., 1998, Eur. J. Biochem., 254:634-642. As evidence shows that endocannabinoids have the ability to influence tumor cell proliferation and cell apoptosis in vivo and in vitro through activating cannabinoid receptors (CB1 and CB2) and vanilloid receptors (Kogan, 2005, Mini Rev. Med. Chem., 5:941-952; Bifulco and DiMarzo, 2002, Nat. Med., 8:547-550), endocannbinoids have been suggested as a potential target for CRC therapy. Patsos et al., 2005, Biochem Soc. Trans., 33:712-714.

The observed differently expressed metabolites in CRC patients using multivariate and univariate statistical significance collectively constitute a metabolic window into CRC morbidity; providing metabolic endpoints that complement the interpretation of genomic, proteomic, and epidemiological data. These results also highlight the potential of this sufficiently robust and non-invasive profiling approach for detection of CRC.

### Example 2

### Metabolite Markers for Colorectal Cancer Identified in Urine (Initial Study)

### Material and Methods for Urine Analysis

Chemicals. Ethyl chloroformate (ECF), pyridine, anhydrous ethanol, sodium hydroxide, chloroform, and anhydrous sodium sulfate were analytical grade from China National Pharmaceutical Group Corporation (Shanghai, China). L-2-chlorophenylalanine was purchased from Intechem Tech. Co. Ltd. (Shanghai, China).

Clinical samples. Urine samples were collected from the same 60 CRC patients and 63 healthy volunteers from Cancer Hospital, Shanghai Medical College, Fudan University (Shanghai, China) as described in Example 1. All subjects signed an informed consent under local research ethics committee approval. The urine samples were taken from most of the human subjects participated in our previous serum metabonomic study. Qiu et al., 2009, J. Proteome Res., 8:4844-4850. All the CRC patients were diagnosed with different histopathological features and stages according to recent TNM classification: stage I, 7 patients; stage II, 23 patients; stage III, 21 patients; stage IV, 9 patients. Body mass index (BMI) and carcinoembryonic antigen (CEA) level for each patient were assessed. More detailed demographic profiles of participants are provided in Table 5. Patients enrolled in this study were subject to surgical operation, and were not on any medication before preoperative sample collection. Liquid paraffin and lactulose oral solution were used to flush the colon two days prior to the surgical operation. In the first 3-5 days after surgical operation, patients were administered an amino acid mixture by intravenous drip containing the following 18 amino acids: L-alanine, L-arginine, L-aspatrate, L-cysteine, glycine, L-histidine, L-isoleucine, L-leucine, L-lysine, L-methionine, L-phenylalanine, L-proline, L-serine, L-threonine, L-tryptophan, L-tyrosine, L-valine and L-glutamate. The postoperative samples were collected on the 7^{th} day after surgery. All urine samples were collected in the morning before breakfast, immediately centrifuged, and stored at −80° C. until analysis.

TABLE 5
Demographic and clinical chemistry
characteristics of human subjects

| | CRC patients | Healthy controls |
|---|---|---|
| n | 60 | 63 |
| Age (median, range) | 58.8, 42-76 | 55.5, 42-74 |
| Male/female ratio | 34/26 | 32/31 |
| BMI (median, range) | 22.1, 16.4-28.9 | 23.3, 17.8-27.4 |
| CEA (>5.0 ng/ml) | 25 | N/A |
| Stage I | 7 | / |
| Stage II | 23 | / |
| Stage III | 21 | / |
| Stage IV | 9 | / |

Animal treatment and sampling. The animal study was conducted in accordance with Chinese national legislation and local guidelines, and performed at the Centre of Laboratory Animals, Shanghai University of Traditional Chinese Medicine, P. R. China. A total of 16 male Wistar rats (90±10 g) were commercially obtained from Shanghai Laboratory Animal Co. Ltd. (SLAC, China) and kept in a barrier system with regulated temperature (23-24° C.) and humidity (60±10%), on a 12/12-hr light/dark cycle (lights on at 08:00 AM), and fed certified standard rat chow and tap water ad libitum. After two weeks acclimatization, the 16 rats were divided randomly into two groups: a 1,2-dimethylhydrazine (DMH) treated (precancerous lesion model) group (n=8), dosed with DMH solution prepared in physiological saline via intraperitoneal (i.p.) injection at 30 mg/kg twice (one week interval); and the healthy control group (n=8), dosed with the same volume of saline via i.p. injection at the same times as the model group. Alkylating agents, such as 1,2-dimethylhydrazine (DMH) and azoxymethane, are often used to produce precancerous colorectal lesions or colorectal cancer animal models. Newell and Heddle, 2004, Mutat. Res., 564:1-7. DMH has been reported to induce DNA mutations and subsequently cause aberrant crypt foci (ACF), which serve as a reliable intermediate biomarker for colorectal carcinogenesis, (Bird, 1995, Cancer Lett., 93:55-71; Bird, 1987, Cancer Lett., 37:147-151) in the colon and rectum. Agner et al., 2005, Mutat. Res., 582:146-154. Urine samples were collected seven weeks after the second DMH injection and centrifuged at 10,000 rpm for 10 minutes to remove suspended debris and stored at −80° C. pending GC-MS analysis. After sample collection, all of the animals were sacrificed by decapitation after halothane anesthesia and subjected to autopsy.

GC-MS spectral acquisition of urine samples. Urine samples from humans and rats were prepared for GC-MS analysis and spectral acquisition with minor modifications to our previously developed method. Qiu et al., 2007, Anal. Chim. Acta, 583:277-287. Briefly, after thaw at room temperature, the urine samples were subjected to a 3,000 g centrifugation for 10 minutes. A typical 600 μL aliquot of supernatant of human urine sample (300 μL rat urine was diluted with 300 μL distilled water) spiked with internal standards (100 μl of L-2-chlorophenylalanine in water, 0.1 mg/mL) was prepared for ECF derivatization. After adding 400 μL of anhydrous ethanol, 100 μL of pyridine, and 50 μL of ECF to the urine sample, the derivatization was conducted at 40 KHz ultrasonication for 60 seconds. The extraction was carried out using 300 μL of chloroform, with the aqueous layer pH carefully adjusted to 9-10 using 100 μL of NaOH (7 mol/L). The derivatization procedure was repeated with another 50 μL ECF into the aforementioned products. After the overall mixtures were vortexed for 30 seconds and centrifuged at 1,900 g for 10 minutes, the aqueous layer was aspirated off, while the remaining chloroform layer containing derivatives was obtained and dried with anhydrous sodium sulfate for subsequent GC-MS analysis.

Each 1 μL chloroform layer containing ECF-derivatives was injected into a DB-5MS capillary column (30 m×250 μm i.d., 0.25 μm film thickness; Agilent J&W Scientific, Folsom, USA) and conducted on a hyphenated PerkinElmer gas chromatography-TurboMass-Autosystem XL mass spectrometer (PerkinElmer Inc., USA). Helium was used as the carrier gas at a constant flow rate of 1.0 ml/min. The injection and transfer interface temperature were both set to 260° C. The GC oven temperature was started at 80° C. for 2 min, then raised to 140° C. with a rate of 10° C./min, followed by 4° C./min to 180° C., and 10° C./min to 280° C. and maintained at 280° C. for 3 min. Electron impact ionization (70 eV) at full scan mode (m/z 30-500) was used, with the ion source temperature at 200° C. Urine samples were analyzed at a CRC-control-CRC (model-control-model for rats) sequence to eliminate any systemic bias, and I-2-chlorophenylalanine was used as an internal standard to monitor batch reproducibility.

Data Analysis. GC-MS raw data files were initially converted into NetCDF format using DataBridge™ software (Perkin-Elmer Inc., USA), then were directly processed using the XCMS Toolbox (XCMS Analyte Profiling Software) (freely available at http://metlin.scripps.edu/download/). Data was processed using XCMS's default settings with the following exceptions: xcmsSet (full width at half-maximum: fwhm=4; S/N cutoff value: snthresh=8, max=20), group (bw=5). Smith et al., 2006, Anal. Chem., 78:779-787. XCMS (various forms of (“X”) chromatography mass spectrometry) is part of the official Bioconductor project. Bioconductor is an open source and open development software project for the analysis and comprehension of genomic data. The xcms package reads and processes LC/MS data stored in NetCDF (AIA/ANDI), mzXML, and mzData files. The software includes numerous options for visualizing and interacting with that data, as well as functionality for peak picking, non-linear retention time alignment, and relative quantitation. It is capable of simultaneously preprocessing, analyzing, and visualizing the raw data from hundreds of samples. The resulting comma separated values (CSV) file was exported into MATLAB 7.0 (The MathWorks, Inc. USA), where exclusion of the internal standard peak, and normalization of the total sum of the chromatogram were performed. The resultant three dimensional matrix encompassing peak indices (retention time-m/z pairs), sample names (observations), and normalized peak areas (variables) were imported into the SIMCA-P 12.0 Software package (Umetrics, Umeå, Sweden). The data was mean-centered and pareto-scaled prior to multivariate statistical analysis.

Unsupervised principal component analysis (PCA) was initially carried out to obtain an overview of urinary GC-MS data from cancer patients or precancerous lesion rats and their healthy control counterparts. However, the PCA results can be influenced by many factors, such as gender, age, and pathological variations. To specify the metabolic variations associated with CRC morbidity, supervised partial least squares (PLS) and orthogonal partial least squares-discriminant analysis (OPLS-DA) were subsequently used.

Differential variables responsible for the deviated metabolic profiles of CRC or precancerous lesion individuals from healthy controls were selected based on a threshold of variable importance in the projection (VIP) value (VIP>1) from a typical 7-fold cross-validated OPLS-DA model. To guard against model over-fitting, the default 7-round cross-validation in SIMCA-P software package was applied with 1/7^{th} of the samples being excluded from the model in each round. A 999 random permutations test was also performed to further validate the supervised model. A 999 permutation test (also known as a randomization test) is a statistical significance test in which a reference distribution is obtained by calculating all possible values of the test statistic under rearrangements of the labels on the observed data points. In other words, the method by which treatments are allocated to subjects in an experimental design is mirrored in the analysis of that design. If the labels are exchangeable under the null hypothesis, then the resulting tests yield exact significance levels. Confidence intervals can then be derived from the tests.

In parallel, these differential metabolites from the OPLS-DA model were validated at a univariate level using student's t test. To obtain a wide range of differential metabolites with large VIP values, the critical p value of the test was set to 0.1 in this study. The corresponding fold change shows how the urinary GC-MS profiles of diseased individuals varied from that of the healthy controls. The box plots of some typical differential metabolites were conducted by SPSS for Windows Software (16.0, Chicago, Ill., USA). Compound identification was performed by comparing the mass fragments with those present in commercially available mass spectral databases such as NIST, Wiley and NBS, with a similarity threshed of 70%. Finally, about half of them were verified by reference compounds.

### Results

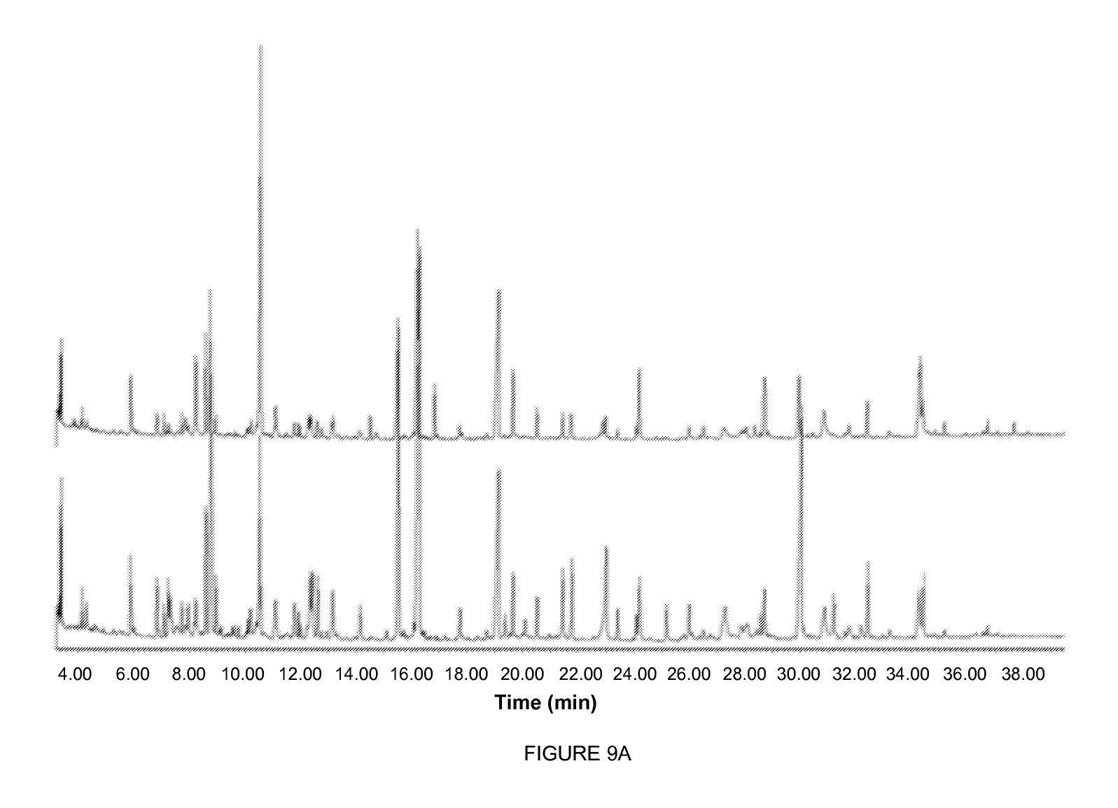

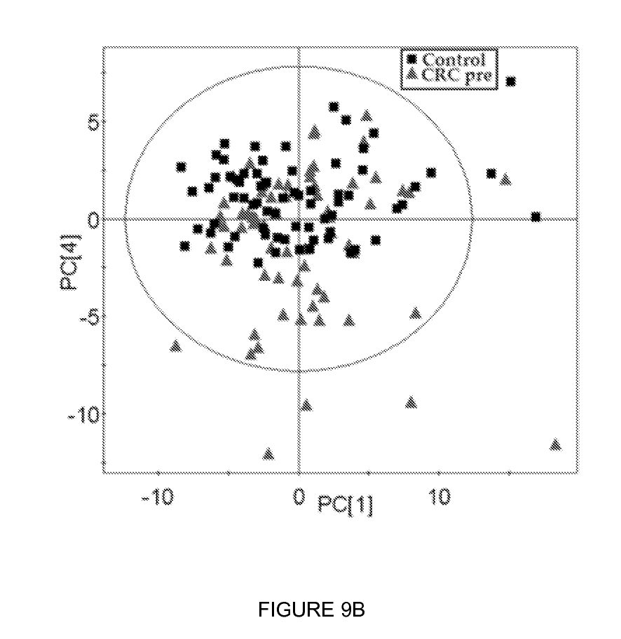

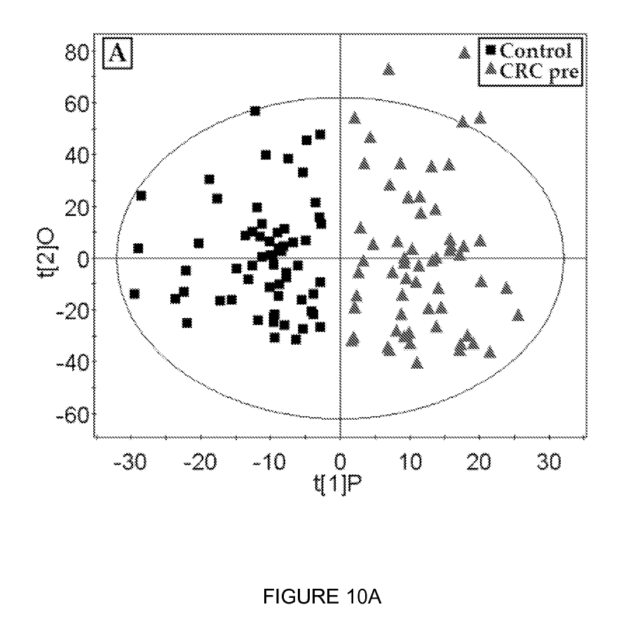

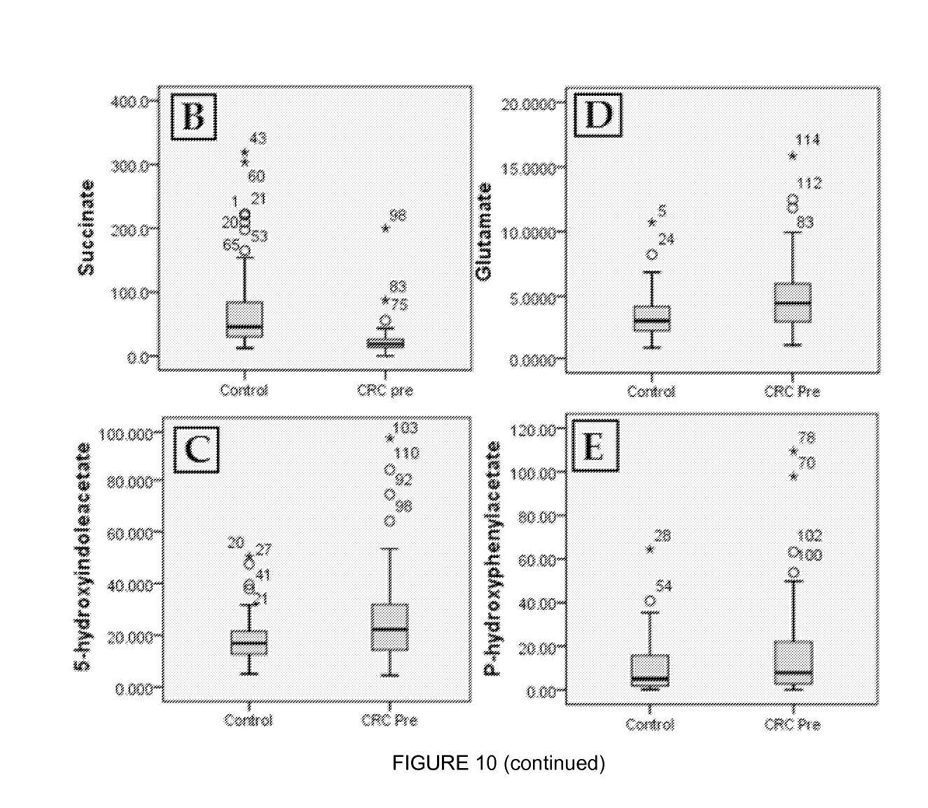

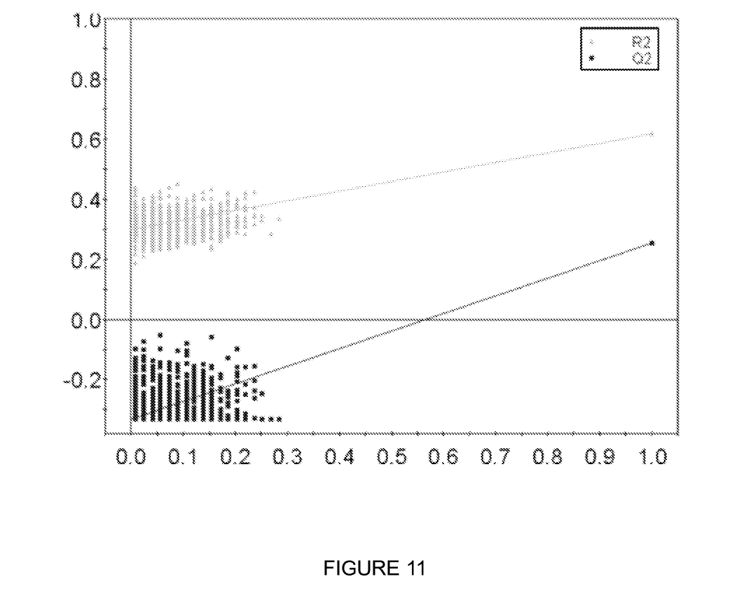

Metabolic profiles between preoperative CRC patients and healthy controls. Typical GC-MS total ion current (TIC) chromatograms of urine samples derived from a preoperative cancer patient and a healthy control are illustrated in FIG. 9A. After excluding internal standards, a total of 187 individual metabolites were consistently detected in nearly 90% of the urine samples, including organic acids, amines, and amino acids. The average relative standard deviation (RSD) of these 187 variables are 84.66%, 93.98%, and 108.71% for the healthy control group, the preoperative samples and the post operative group, respectively, suggesting a consistent intra-group variation among the three groups. PCA scores plot based on the resulting 187 variables using 5 components (R²Xcum=0.343, Q²cum=0.187) showed a separation tendency from preoperative CRC patients and healthy controls FIG. 9B. In order to specify cancer-related metabolic variations, a cross-validated OPLS-DA model was constructed with satisfactory predictive ability using one predictive component and three orthogonal components (R²Xcum=0.4, R²Xp=0.119, R²Ycum=0.763, Q²Ycum=0.467) (FIG. 10A). In the permutation test, all the R² (cum) and Q2 (cum) values calculated from the permuted data were lower than the original ones in the validation plot. The Q2 (cum) intercepted the y-axis at −0.334 (FIG. 11). These results assured the validity of the OPLS-DA model between preoperative CRC patients and healthy controls. All the cancer patients were completely discriminated from the healthy controls in the predictive component, including seven patients diagnosed at TNM stage I. This result indicates great potential for early diagnosis of CRC using the current, noninvasive urinary metabonomic analysis. However, similar to the serum metabonomics study of Example 1, further classification of CRC patients based on their different pathological stages using PLS or OPLS-DA models of urinary metabolite profiles was not conducted. Attempts were made to stratify patients with different CEA levels using metabonomic data, but no valid model of metabonomic profile differentiation was established between CRC patients with high CEA (>5 ng/mL) and CRC patients with low CEA levels.

Based on the OPLS-DA results with a good group classification between 60 CRC patients and 63 controls, a total of 36 paired retention time-mass to charge ratio (RT-M/Z) variables were selected according to the VIP threshold (VIP>1). A list of 16 differential metabolites was identified by library search, and 9 metabolites were verified by the available reference compounds (Table 6). To further interpret the biological significance associated with CRC morbidity, the Kyoto Encyclopedia of Genes and Genomes (KEGG) database was used to link these metabolites to metabolic pathways. Williamson et al., 1977, Clin. Sci. Mol. Med., 52:P17-P18. The main metabolic pathways involved in CRC patients were TCA cycle, histamine metabolism, glutamate metabolism, tryptophan metabolism and the altered structure of gut flora. Box plots of succinate, 5-hydroxyindoleacetate, glutamic acid, and 2-hydroxyhippurate, which correlated with four disturbed metabolic pathways, are provided in FIG. 10B to 10E, in order to demonstrate the individual metabolite differences between CRC patients (preoperative) and healthy controls. Six metabolites with characteristic expression levels were identified among different CRC stages (FIG. 11; Table 7).

TABLE 6
A list of identified differential metabolites between
preoperative CRC patients and healthy controls

| | | | Fold | | |
|---|---|---|---|---|---|
| | | | Change^{c} | | |
| | | | (CRC/ | | Metabolic |
| No. | VIP^{a} | p^{b} | control) | metabolite | pathways |
| 1 | 3.77 | 9.3e−7 | −2.9 | Succinate^{d} | TCA cycle |
| 2 | 1.76 | 0.0123 | −1.3 | Isocitrate^{d} | TCA cycle |
| 3 | 1.04 | 0.0432 | −1.2 | Citrate^{d} | TCA cycle |
| 4 | 1.65 | 0.0005 | 2.0 | 5-hydroxytryptophan^{d} | Tryptophan |
| | | | | | metabolism |
| 5 | 1.44 | 0.0070 | 1.5 | 5- | Tryptophan |
| | | | | hydroxyindoleacetate^{d} | metabolism |
| 6 | 2.48 | 0.0308 | 1.2 | Tryptophan^{d} | Tryptophan |
| | | | | | metabolism |
| 7 | 5.79 | 0.0014 | 2.0 | Glutamate | Glutathione |
| | | | | | metabolism |
| 8 | 2.07 | 0.0203 | 1.2 | 5-oxoproline | Glutathione |
| | | | | | metabolism |
| 9 | 1.32 | 1.6e−6 | 1.9 | N-acetyl-aspartate | Aspartic |
| | | | | | acid |
| | | | | | metabolism |
| 10 | 1.51 | 0.0582 | −1.3 | 3-methyl-histidine | Degradation |
| | | | | | of actin and |
| | | | | | myosin |
| 11 | 1.77 | 0.0601 | −1.1 | Histidine^{d} | Histamine |
| | | | | | metabolism |
| 12 | 2.27 | 0.0961 | 1.4 | p-cresol^{d} | Gut flora |
| | | | | | metabolism |
| 13 | 1.99 | 0.0004 | 1.8 | 2-hydroxyhippurate | Gut flora |
| | | | | | metabolism |
| 14 | 1.61 | 0.0875 | 1.2 | Phenylacetate^{d} | Gut flora |
| | | | | | metabolism |
| 15 | 1.80 | 0.0231 | 1.4 | Phenylacetylglutamine | Gut flora |
| | | | | | metabolism |
| 16 | 1.30 | 0.0304 | 2.6 | p- | Gut flora |
| | | | | hydroxyphenylacetate | metabolism |

^{a}Variable importance in the projection (VIP) was obtained from OPLS-DA with a threshold of 1.0.
^{b}p-value was calculated from paired student t test.
^{c}Fold change was calculated from the arithmetic mean values of each group. Fold change with a positive value indicates a relatively higher concentration present in CRC patients, while a negative value means a relatively lower concentration as compared to the healthy controls.
^{d}Metabolites verified by reference compounds, other were directly obtained from library searching.

TABLE 7
Mean (Mean ± SD) values of six metabolites showed
characteristic expression levels among different CRC stages.

| | Stage I | Stage II | Stage III | Stage IV |
|---|---|---|---|---|
| | (mean ± SD) | (mean ± SD) | (mean ± SD) | (mean ± SD) |
| Indoleacetate | 1.94 ± 1.76 | 0.64 ± 0.35 | 0.83 ± 0.66 | 0.60 ± 0.47 |
| P-hydroxy- | 0.97 ± 1.62 | 1.72 ± 3.83 | 0.64 ± 0.63 | 0.68 ± 0.68 |
| phenylacetate | | | | |
| 5-hydroxy- | 0.70 ± 0.36 | 1.12 ± 0.56 | 1.51 ± 1.53 | 0.67 ± 0.45 |
| indoleacetate | | | | |
| 2- | 1.07 ± 2.5 | 1.53 ± 3.54 | 1.31 ± 3.64 | 0.09 ± 0.07 |
| methyl- | | | | |
| propanoate | | | | |
| Glutamate | 0.56 ± 0.25 | 0.88 ± 1.05 | 1.24 ± 1.60 | 1.32 ± 1.16 |
| Leucine | 2.11 ± 1.39 | 0.80 ± 1.20 | 0.74 ± 1.26 | 0.36 ± 0.76 |

The values were normalized to the mean value of all the patients within each metabolite.

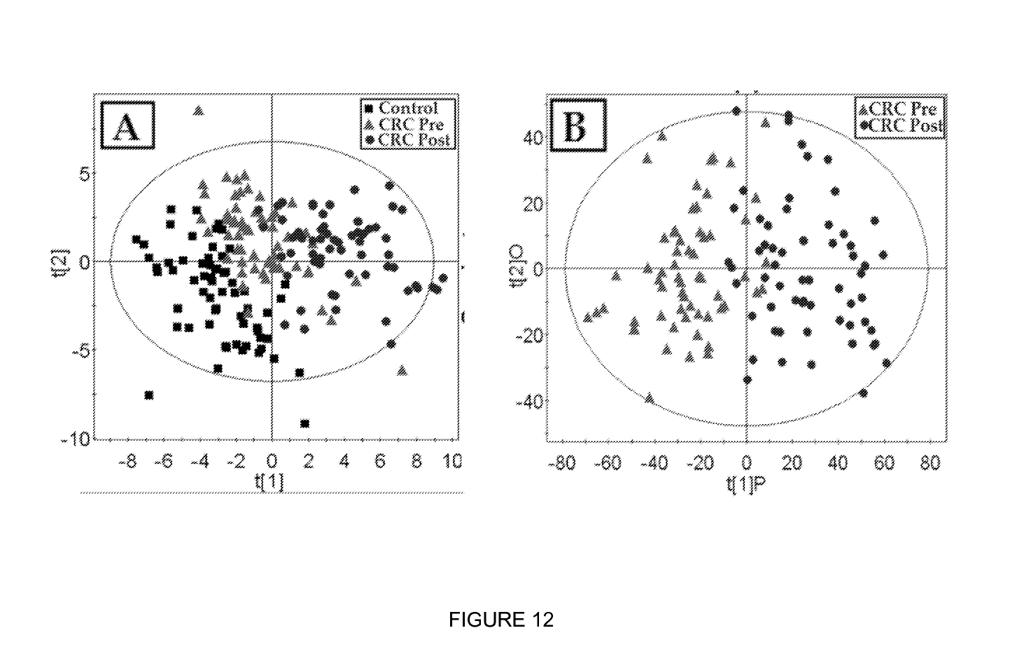

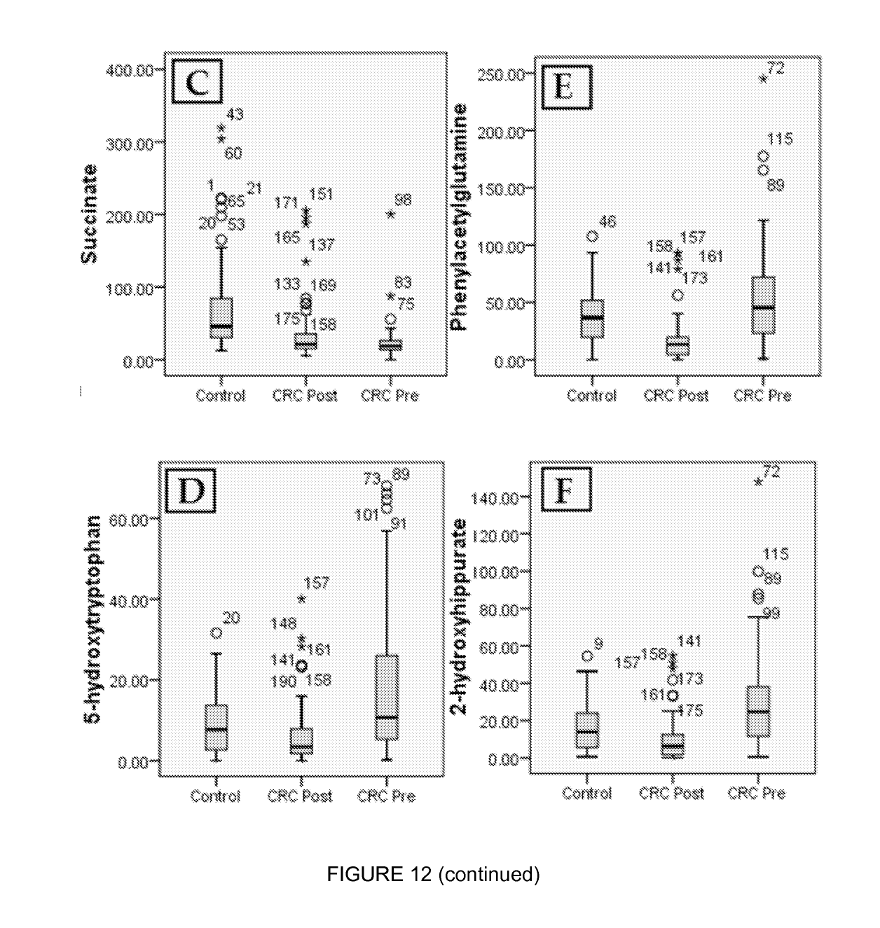

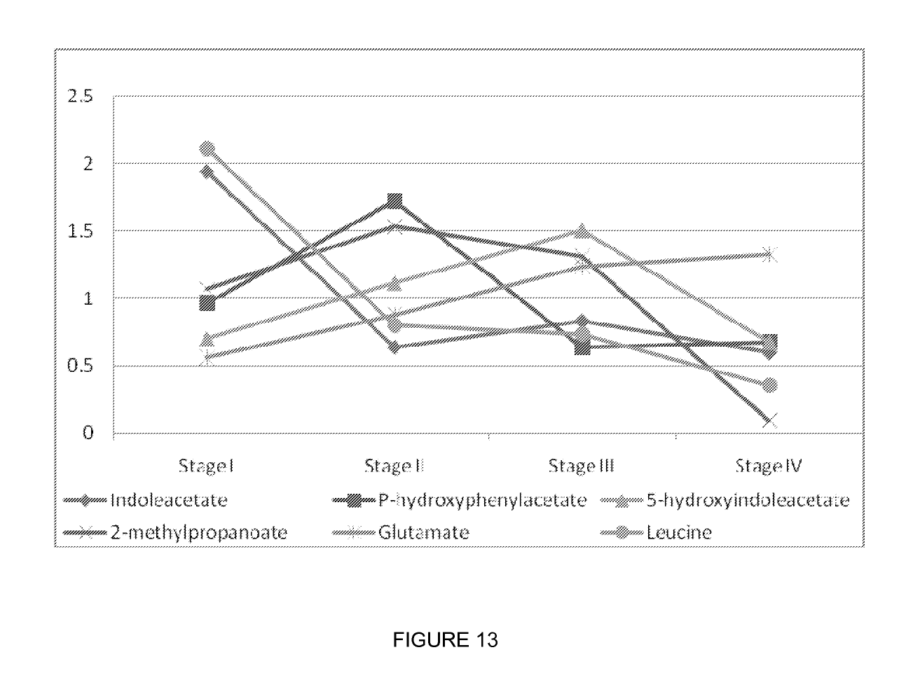

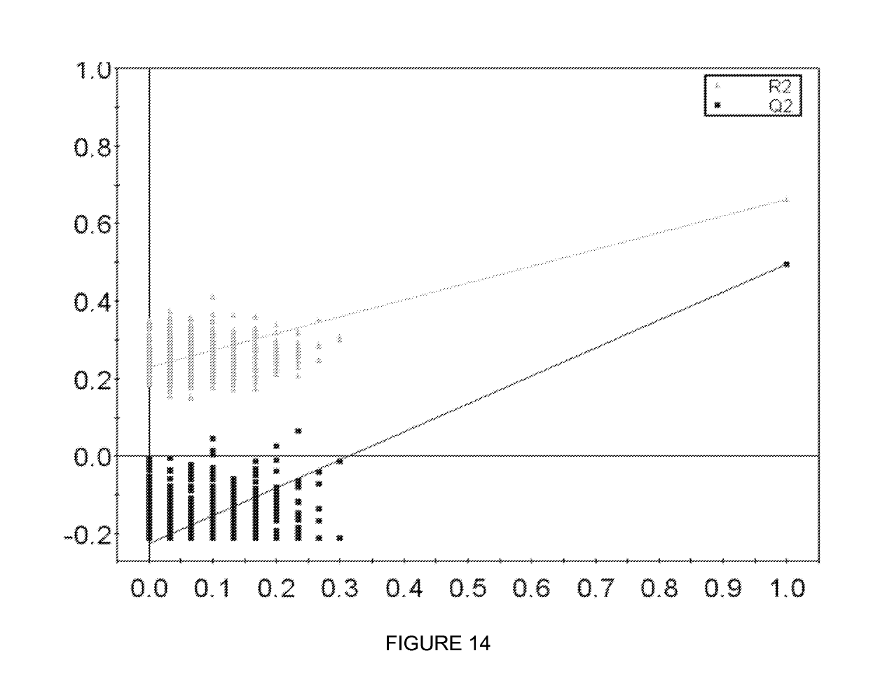

The metabolite variations in post operative CRC patients. A PLS scores plot of metabonomic profiles from controls and CRC patients (preoperative and postoperative) was constructed in FIG. 12A (three components, R²Xcum=0.194, R²Ycum=0.694, Q²Ycum=0.554). Separations can be achieved among these three groups, especially between control samples to the other two groups. Furthermore, the OPLS-DA scores plot was also able to demonstrate a good separation between preoperative and postoperative samples using one predictive component and one orthogonal components (R²Xcum=0.198, R²Xp=0.146, R²Ycum=0.662, Q²Ycum=0.525) (FIG. 12B). The permutation test indicated the model is not over-fitting (FIG. 14). Based on the VIP values and paired student's t test, 45 differential variables were selected, among which 21 were identified (Table 8). Most of them were amino acids and phenyl-containing metabolites. The variations of the significantly altered metabolites in preoperative CRC patients relative to controls were also investigated. Among those 16 identified metabolites, four of them (succinate, phenylacetylglutamine, 2-hydroxyhippurate, and 5-hydroxytryptophan) showed a recovering tendency towards healthy state in the post operative samples (FIG. 12C to 12F; Table 9).

TABLE 8
Differential metabolites between pre- and post-operative
CRC patient samples.

| | | | fold | | |
|---|---|---|---|---|---|
| | | | change^{c} | | |
| | | | (CRC/ | | Metabolic |
| No. | VIP^{a} | p^{b} | control) | metabolite | pathways |
| 1 | 1.76 | 0.0024 | −1.3 | Aconitate^{d} | TCA cycle |
| 2 | 3.81 | 0.0019 | −1.3 | Citrate^{d} | TCA cycle |
| 3 | 1.21 | 0.0024 | 1.3 | 5-oxoproline | Glutathione |
| | | | | | metabolism |
| 4 | 3.45 | 1.0e−6 | 1.6 | Tryptophan^{d} | Tryptophan |
| | | | | | metabolism |
| 5 | 1.73 | 0.0396 | 1.3 | Histidine^{d} | Histamine |
| | | | | | metabolism |
| 6 | 2.11 | 0.0039 | 1.6 | Tyrosine^{d} | Tyrosine |
| | | | | | metabolism |
| 7 | 2.50 | 0.0364 | 1.7 | Lysine^{d} | Lysine |
| | | | | | metabolism |
| 8 | 1.86 | 2.0e−5 | 1.9 | Asparagine | Aspartate |
| | | | | | Metabolism |
| 9 | 1.27 | 0.0020 | 2.3 | Threonine^{d} | Threonine |
| | | | | | Metabolism |
| 10 | 1.13 | 2.6e−5 | 1.5 | Leucine^{d} | Leucine |
| | | | | | degradation |
| 11 | 1.70 | 0.0008 | 1.3 | Isoleucine^{d} | Isoleucine |
| | | | | | degradation |
| 12 | 2.10 | 0.0003 | 1.6 | Serine^{d} | Serine |
| | | | | | Metabolism |
| 13 | 2.77 | 0.0002 | −3.9 | p-cresol^{d} | Gut flora |
| | | | | | metabolism |
| 14 | 8.32 | 9.6e−9 | −4.8 | Hippurate^{d} | Gut flora |
| | | | | | metabolism |
| 15 | 1.05 | 3.9e−5 | −2.1 | Phenylacetate^{d} | Gut flora |
| | | | | | metabolism |
| 16 | 1.12 | 3.8e−6 | −3.1 | 2-hydroxyhippurate | Gut flora |
| | | | | | metabolism |
| 17 | 1.69 | 5.1e−7 | −3.4 | Phenylacetylglutamine | Gut flora |
| | | | | | metabolism |
| 18 | 1.46 | 0.0010 | −2.8 | Methyl o-hydroxy- | Gut flora |
| | | | | hippurate | metabolism |
| 19 | 1.67 | 0.0839 | 1.15 | Aminomalonate | Others |
| 20 | 1.16 | 0.0123 | −7.6 | Pentanedioate | Others |
| 21 | 1.46 | 2.8e−9 | 2.4 | Glycylproline | Others |

^{a}Variable importance in the projection (VIP) was obtained from OPLS-DA with a threshold of 1.0.
^{b}p-value was calculated from paired student t test.
^{c}Fold change was calculated from the arithmetic mean values of each group. Fold change with a positive value indicates a relatively higher concentration present in 1,2-dimethylhydrazine (DMH)-treated Sprague-Dawley CRC model rats while a negative value means a relatively lower concentration as compared to the healthy controls.
^{d}Metabolites verified by reference compounds, other were directly obtained from library searching.

TABLE 9
Comparison of the mean values, standard deviations (SDs) and the p values from student
t test of four differential metabolites showing alteration of postoperative samples (Post-Op)
values towards values of healthy controls as compared to preoperative samples (Pre-Op).

| | | | | p | p | p |
|---|---|---|---|---|---|---|
| | | | | values^{a} | values^{a} | values^{a} |
| | Mean ± SD | Mean ± SD | Mean ± SD | Pre-Op | Pre-Op | Post-Op |
| | control | Pre-Op | Post-Op | vs. | vs. | vs. |
| Metabolites | (n = 63) | (n = 60) | (n = 60) | Control | Post-Op | Control |
| Succinate | 70.51 ± 66.45 | 24.12 ± 20.31 | 35.57 ± 23.01 | 9.3E−07 | 0.0698 | 0.0005 |
| Phenylacetyl- | 38.34 ± 25.32 | 53.42 ± 46.19 | 17.53 ± 16.02 | 0.0231 | 1.1E−07 | 1.8E−06 |
| glutamine | | | | | | |
| 2-hydroxy- | 16.17 ± 12.44 | 29.83 ± 27.19 | 10.08 ± 9.71 | 0.0003 | 6.1E−07 | 0.0092 |
| hippurate | | | | | | |
| 5-hydroxy- | 9.09 ± 7.51 | 18.26 ± 16.15 | 6.67 ± 6.19 | 0.0005 | 1.9E−05 | 0.1193 |
| tryptophan | | | | | | |

^{a}p values were calculated based on student t test.

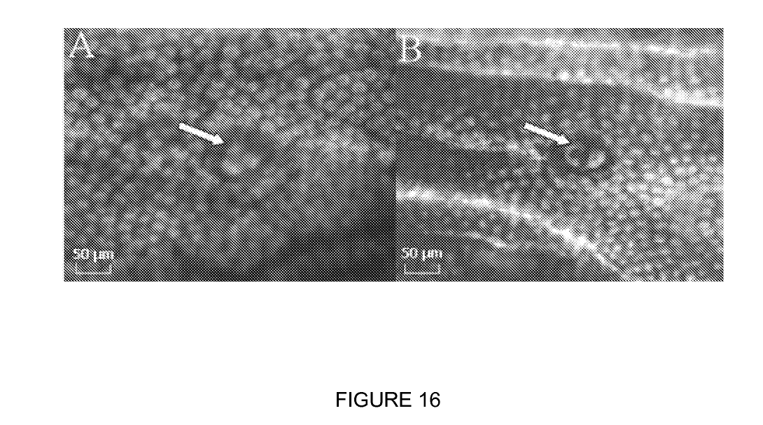

Metabolic profiles between model rats and controls. A sharply increased ACF number (37.7±2.6) was observed microscopically in the DMH-treated precancerous colorectal lesion model rat colons and rectums, as compared with that of healthy controls (the number of ACF is 0) at post-dose week 7. Typical histological ACF lesions are shown in FIGS. 16A and 16B; which confirm that the precancerous colorectal lesion rat model was successfully produced in the current experiment.

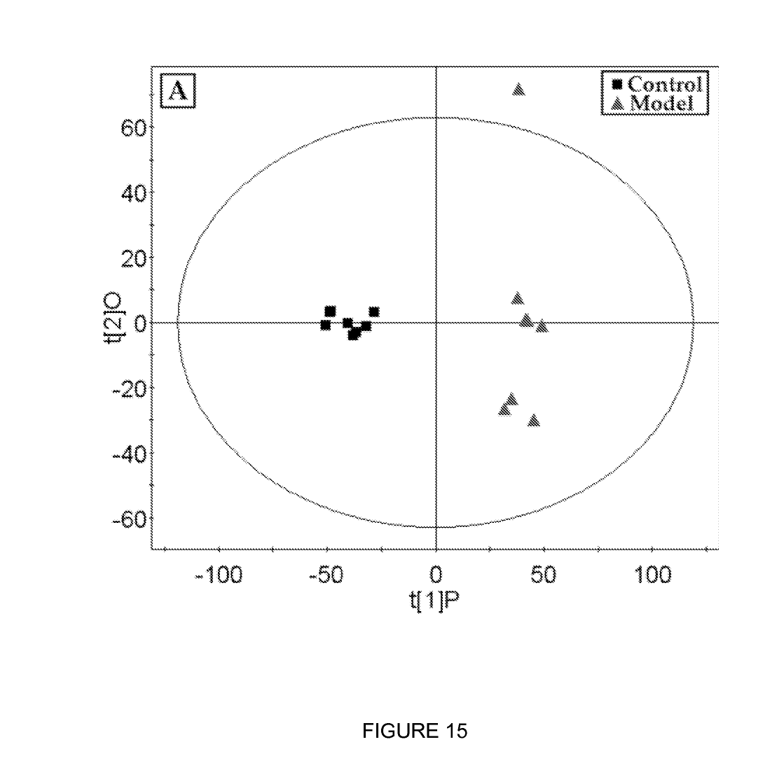

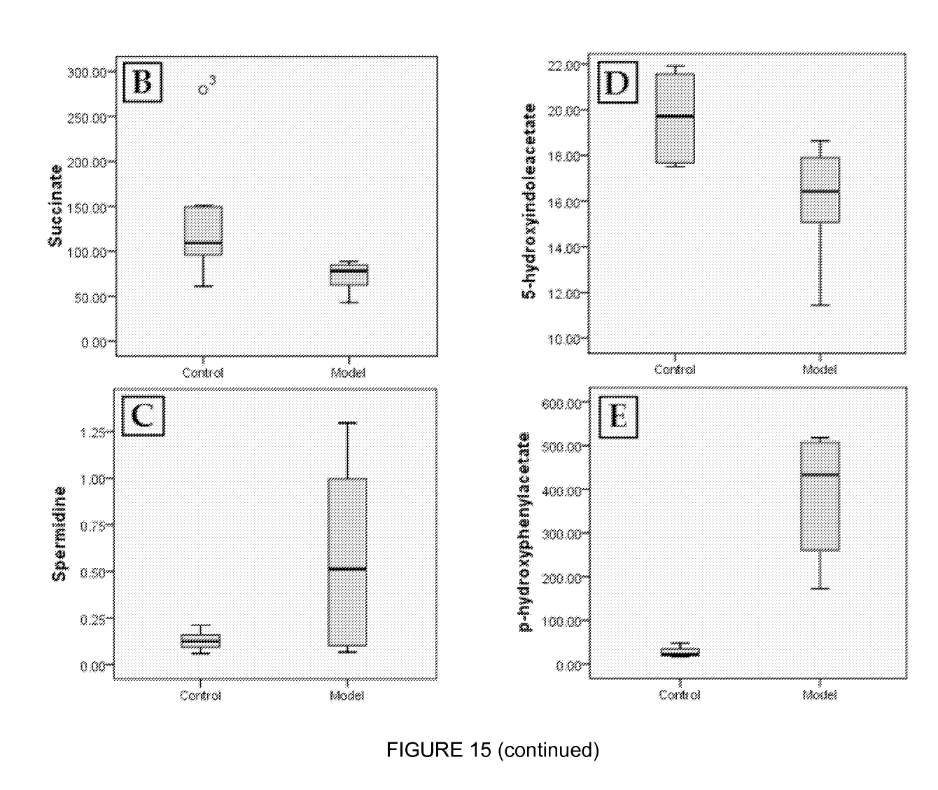

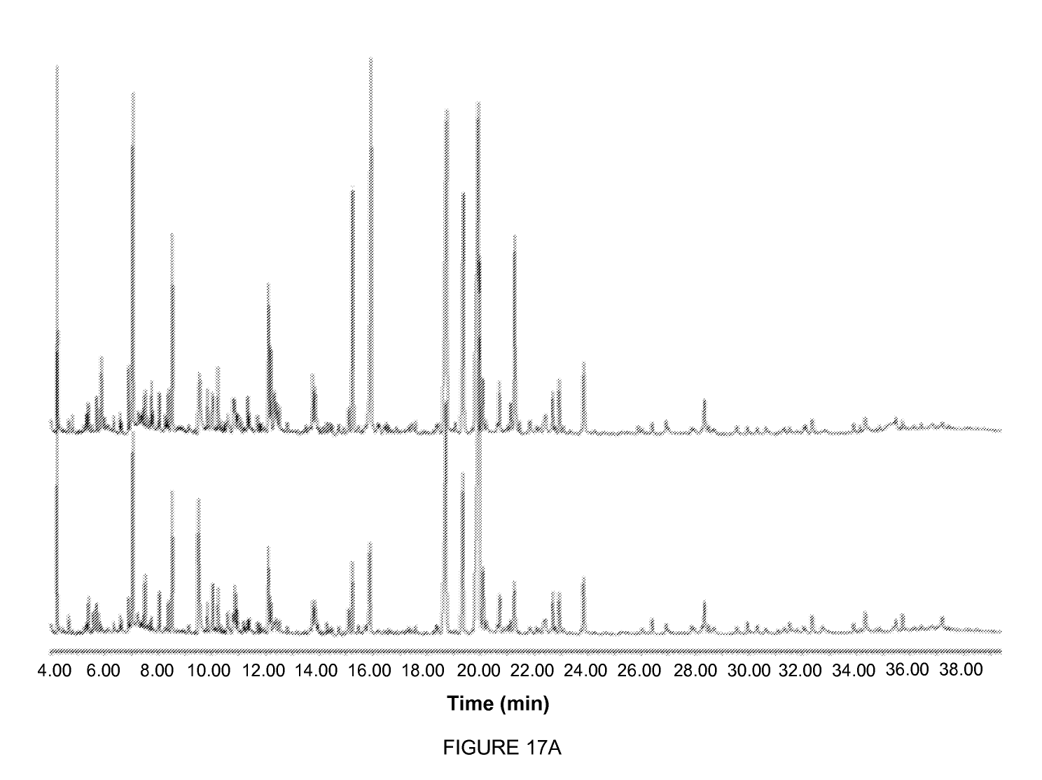

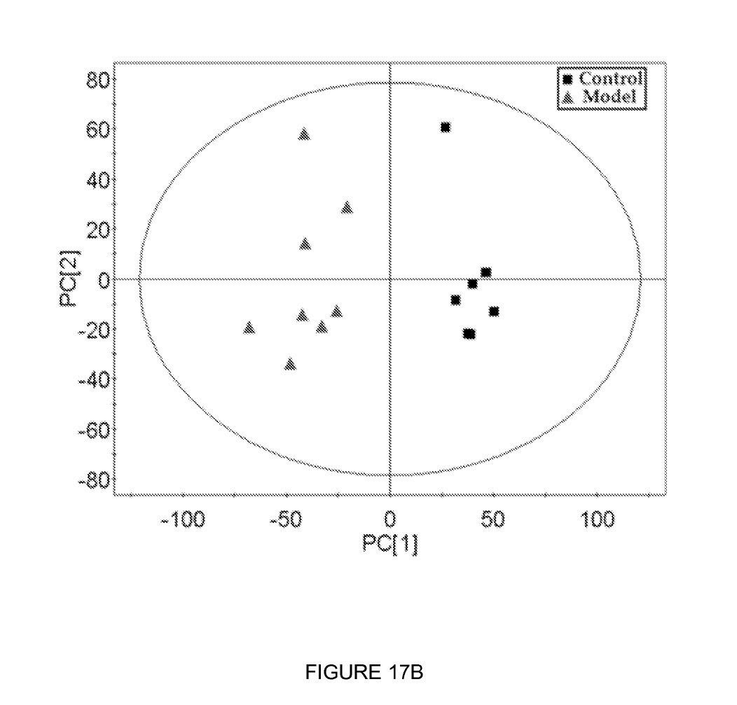

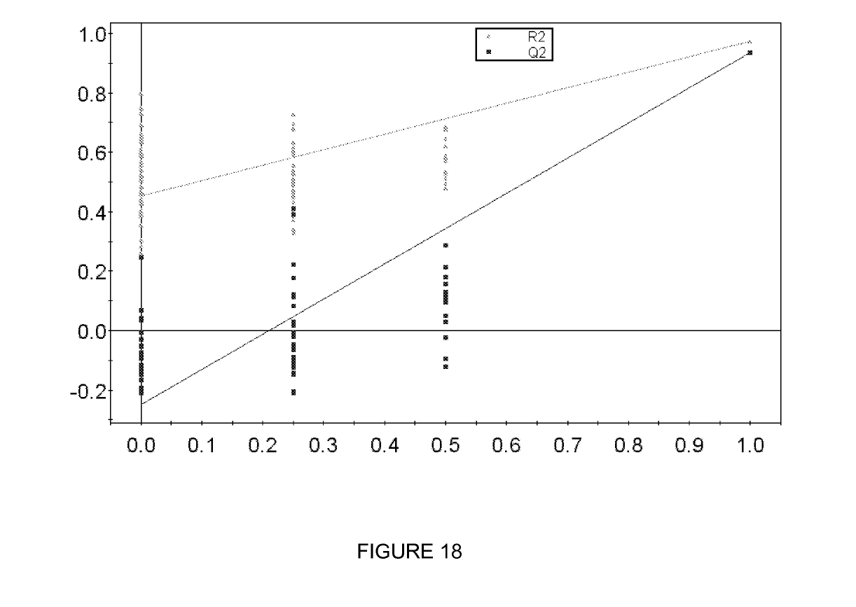

GC-MS TIC chromatograms of urine samples deriving from a DMH-induced rat and a healthy control rat are shown in FIG. 17A. A total of 215 individual variables were detected in the rat urine samples, in addition to the internal standard. PCA scores plot showed clear separation between model rats and controls (four components with R²Xcum=0.839, Q²cum=0.279). To obtain the metabolic differences accounting for pathological influence, a supervised OPLS-DA model was carried out. Distinct differentiation was also found in the OPLS-DA scores plot (FIG. 15A) between model rats and the healthy controls, with a high value of R²Y or Q²Y for the model using one predictive component and one orthogonal components (R²Ycum=0.571, R²Xp=0.437, R²Ycum=0.974, Q²Ycum=0.934). This model was also validated by the permutation test (see FIG. 18).

A total of 41 of the most significant variables contributing to class separation were selected from the cutoff value of VIP (VIP>1). Using GC-MS spectral databases, fifteen metabolites were identified (Table 10), nine of which were verified by standard compounds. Seven of the fifteen identified metabolites were also identified in the differential variables between preoperative CRC patient and healthy human samples: p-cresol, succinate, phenylacetate, isocitrate, citrate, p-hydroxyphenylacetate and 5-hydroxyindoleacetate. By connecting the individual metabolites with metabolic pathways in KEGG database, those differential metabolites included intermediates of the TCA cycle at significantly elevated levels: succinate, citrate, aconitate and isocitrate; tryptophan metabolism: indoleacetate, tryptamine and 5-hydroxyindoleacetate; and polyamines: putrescine and spermidine. Additionally, some phenyl containing metabolites, generally believed to be metabolized via gut flora, were also observed in the model rat urine compared with the healthy control, including significantly down-regulated p-cresol, hippurate, phenylacetate, m-hydroxyphenylpropionate and up-regulated p-hydroxyphenylacetate and phenylacetylglycine. Box plots for typical differential metabolites, succinate, 5-hydroxyindoleacetate, spermidine and p-hydroxyphenylacetate from different metabolic pathways are shown in FIGS. 15B to 15E.

TABLE 10
Differential metabolites between CRC precancerous
colon lesion model and healthy control rats.

| | | | FC^{c} | | |
|---|---|---|---|---|---|
| | | | (model/ | | Metabolic |
| No. | VIP^{a} | p^{b} | control) | metabolite | pathways |
| 1 | 2.18 | 0.0266 | 1.8 | Succinate^{d} | TCA cycle |
| 2 | 1.85 | 0.0390 | 2.5 | Isocitrate^{d} | TCA cycle |
| 3 | 2.17 | 0.0424 | 1.8 | Citrate^{d} | TCA cycle |
| 4 | 1.95 | 0.0634 | 2.0 | Aconitate^{d} | TCA cycle |
| 5 | 2.11 | 0.0063 | 1.9 | 5-hydroxyindole- | Tryptophan |
| | | | | acetate^{d} | metabolism |
| 6 | 1.14 | 0.0031 | 2.0 | Indoleacetate^{d} | Tryptophan |
| | | | | | metabolism |
| 7 | 1.31 | 0.0046 | 2.3 | Tryptamine | Tryptophan |
| | | | | | metabolism |
| 8 | 4.08 | 0.0009 | 2.8 | Putrescine^{d} | Polyamine |
| | | | | | metabolism |
| 9 | 2.38 | 0.0270 | 2.8 | Spermidine^{d} | Polyamine |
| | | | | | metabolism |
| 10 | 1.64 | 0.0259 | −2.2 | p-cresol | Gut flora |
| | | | | | metabolism |
| 11 | 7.26 | 0.0001 | −2.6 | Hippurate^{d} | Gut flora |
| | | | | | metabolism |
| 12 | 1.03 | 0.0810 | 1.6 | Phenylacetyl- | Gut flora |
| | | | | glycine | metabolism |
| 13 | 3.21 | 3.6e−6 | 2.2 | p-hydroxyphenyl- | Gut flora |
| | | | | acetate | metabolism |
| 14 | 1.48 | 0.0301 | −2.0 | Phenylacetate | Gut flora |
| | | | | | metabolism |
| 15 | 4.27 | 0.0566 | −2.5 | M-hydroxyphenyl- | Gut flora |
| | | | | propionate | metabolism |

^{a}Variable importance in the projection (VIP) was obtained from OPLS-DA with a threshold of 1.0.
^{b}p-value was calculated from student t test.
^{c}Fold change was calculated from the arithmetic mean values of each group. Fold change with a positive value indicates a relatively higher concentration present in model rats while a negative value means a relatively lower concentration as compared to the healthy controls.
^{d}Metabolites verified by reference compounds, other were directly obtained from library searching.

### Discussion

All the preoperative CRC patients, including 7 stage I patients, and precancerous colorectal lesion rats were distinguished by these experiments from their healthy counterparts in a OPLS-DA analysis of GC-MS urinary metabolite spectra. Sixteen up- and down-regulated metabolites were identified between CRC patients and the healthy control subjects, while fifteen differential metabolites were identified between model rats and control rats. Seven of the identified metabolites were found in both human samples and rat samples. Furthermore, distinct separation was also achieved between the urine metabonomic profiles from the same cohort of CRC patients in their preoperative and postoperative states. Twenty one differential metabolites were identified which are highly associated with the metabolic changes resulting from the surgical operation.

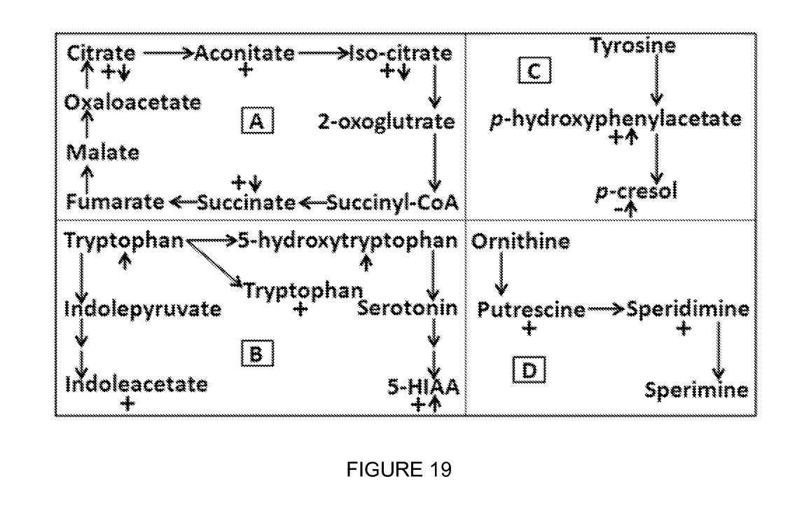

Of the 16 identified differential metabolites in CRC patients, succinate, an important intermediate in the TCA cycle (FIG. 19A), was the metabolite with the most down regulated fold change (−2.9). This suggests that TCA cycle is down-regulated in CRC patients, as evidenced by significantly decreased level of isocitrate and citrate, two other intermediates in the TCA cycle. In the rat studies, however, succinate, isocitrate, citrate and aconitate were found up-regulated in the colorectal lesion rats. The abnormal expression of these metabolites in urine suggests a close association of TCA cycle with CRC morbidity accompanied by disordered aerobic respiration and mitochondrial functions (the main organelle for TCA cycle). The disorder of aerobic respiration (mainly TCA cycle) and the impairment of mitochondrial enzymes have been studied in some cancers, including CRC. Halabe, 2007, Med. Hypotheses, 69:826-828; Bi et al., 2006, Mol. Cell. Proteomics, 5:1119-1130. The down regulation of the TCA in human CRC subjects may be due to the energy consumption status after the onset of CRC and at its several pathological stages. For example, weakened physical condition has previously been reported in 43% subjects in a study of 295 CRC patients, Funch, 1988, Med. Care, 26:1000-1008. In the instant investigation, physical condition of individuals was not record. However, no evident physical activity decrease was noticed in the precancerous model rats, which suggests that the energy consumption with increased TCA cycle is needed along with the development of colon lesion. In addition, as glucose is the main source of glycolysis and TCA cycle, the higher levels of glycolysis in these CRC patients, as described in Example 1, may be another cause of the reduced levels of TCA intermediates in the patient urine. Qiu et al., 2009, J. Proteome Res., 8:4844-4850.

Several differentially expressed metabolites in the tryptophan metabolism (FIG. 19B) including tryptophan, 5-hydroxytryptophan, 5-hydroindoletate and tryptamine were elevated both in CRC patients and CRC model rats compared with their controls. As the precursor, or the product of serotonin, the disordered expression of these metabolites may indicate the disruption of serotonin levels associated with CRC morbidity. As described in Example 1, a significantly lower expression level of tryptophan was observed in serum of the same group of CRC subjects. Qiu et al., 2009, J. Proteome Res., 8:4844-4850. Serotonin has been reported to promote cell proliferation in colorectal carcinomas and selective serotonin reuptake inhibitors were recently reported to reduce the risk of CRC in a nested case-control study. Xu et al., 2006, Lancet Oncol., 7:301-308; Tutton and Barkla, 1982, Cancer, 46:260-265. A previous study also reported elevated levels of 5-hydrotryptophan and 5-hydroindoleacetate in patients with foregut carcinoid tumors. Granerus and Ahlman, 1993, Agents Actions, 38 (Special Issue II). Therefore, up-regulation of tryptophan metabolism may be highly associated with CRC morbidity.

From those differential metabolites identified, prominent variations in the levels of many phenyl-containing compounds, such as p-hydroxyphenylacetate, were elevated both in CRC patients and rat model, p-cresol and phenylacetate were elevated in CRC patients and decreased in rat model, as compared with healthy controls. These metabolites are mainly produced in gut microbiota through fermentation of dietary polyphenols and aromatic amino acids. Rechner et al., 2004, Free Radic. Biol. Med., 36:212-215. For example, p-cresol and p-hydroxyphenylacetate are the metabolites of tyrosine (FIG. 19C) when fermented by C. difficile, a Clostridium species widely distributed in the gut. Stella et al., 2006, J. Proteome Res., 5:2780-2788; Selmer and Andrei, 2001, Eur. J. Biochem., 268:1363-1372. The opposite expression direction of p-cresol between patients (down-regulated) and rat model (up-regulated) suggests that the metabolism from tyrosine to p-cresol by the gut flora may involve multiple pathways. This is consistent with the report that several Clostridium species produce p-hydroxyphenylacetate from tyrosine, but do not decarboxylate it to p-cresol. Elsden et al., 1976, Arch. Microbiol., 107:283 Z-8. The higher level of p-hydroxyphenylacetate and the lower level of p-cresol in the urine may be an indication of the changed population of Clostridium species in the gut associated with the development of CRC morbidity. This is evidenced by an increased diversity and reduced stability of members of the Clostridia as observed in the cultured fecal samples from CRC patients by Scanlan et al., 2008, Environ. Microbiol., 10:1382. Additionally, it has been reported that the induction of CRC by genotoxic carcinogens such as DMH and N-methyl-N′-nitro-N-nitrosoguanidin (MNNG) can severely impact gut flora. Wollowski et al., 2001, Am. J. Clin. Nutr., 73:451 S-4515S. The significantly altered metabolic signatures as reflected by those phenylic compounds in urine clearly indicate a modulated gut microbial metabolism associated with CRC morbidity.

Apart from the three shared metabolic pathways, there were other differential metabolites observed either in humans or in rats. Histidine was found down regulated in CRC patients compared with healthy controls. This may be caused by the higher activity of histidine decarboxylase, thereby accelerating decarboxylation of histidine to histamine in the CRC patients. Garcia-Caballero et al., 1988, Agents Actions, 23:357-360. Glutamate and 5-oxoproline (also known as pyroglutamate) were significantly elevated in CRC patients, which further supports the possible correlation between CRC morbidity and glutamate metabolism, as previously identified in our serum study. Qiu et al., 2009, J. Proteome Res., 8:4844-4850. On the other hand, pyroglutamate is an important intermediate of the γ-glutamyl cycle of glutathione synthesis and degradation. Meister and Anderson, 1983, Annu. Rev. Biochem., 52:711-760. As a naturally occurring cellular antioxidant to detoxify reactive oxygen metabolites, glutathione is involved in the initiation and development of cancers. Balendiran et al., 2004, Cell Biochem. Funct., 22:343-352. Studies revealed that glutathione S-transferase Pi (GSTP1) is highly expressed in cancer tissues including colon cancer (Howie et al., 1990, Carcinogenesis, 11:451-458) and can influence cell apoptosis by regulating Jun N-terminal kinase (JNK) signaling system. Adler et al., 1999, EMBO J., 18:1321-1334.

Two polyamines, putrescine and spermidine, which are produced from ornithine (FIG. 19D), were up-regulated in CRC model rats. Polyamines may modulate the RNA expression of the cancer-related gene cyclooxygenase-2 (COX-2) through the polyamine-dependent gene, elF 5A. Parker and Gerner, 2002, Biochimie, 84:815-819. The elevated urinary excretion levels of putrescine and spermidine from the DMH-treated rats suggest an association between DMH-induced precancerous colorectal lesions and enhanced COX-2 enzyme activity.

Metabolic variations in CRC patients post-surgery compared with their preoperative CRC samples and healthy controls was also investigated. As shown in Table 8, the differential metabolites mainly include up-regulated amino acids, down-regulated TCA intermediates, and down-regulated gut flora metabolism. Nine amino acids such as tryptophan, histidine and tyrosine were observed with higher expression levels in the post operative patient urine. The increased level of these amino acids is mainly due to the nutritional supplementation (containing 18 amino acids for 3-5 days) after the surgery, see Table 11. The other possible cause for the higher amino acids levels is the increased muscle protein breakdown in the post operative patients, since a slightly higher level of 3-methyl-histidine, a marker of muscle protein breakdown (Williamson et al., 1997, Clin. Sci. Mol. Med., 52:P17-P18), was observed in the post operative urine samples (pre-38.99±27.50 VS post 49.25±34.61). The significantly decreased level of citrate and aconitate may be an indication of down-regulated energy metabolism in the post operative patients. An exception is the slightly higher level of succinate, which may be produced from the upstream production of certain amino acids such as alanine and glutamate. The significantly down-regulated gut flora metabolites, as listed in Table 8, are resulted from the colon flush operation prior to surgery. Increased level of 5-oxoproline was observed in the post operative urine samples. As mentioned before, 5-oxoproline is an intermediate of the antioxidant, glutathione, the higher level of this metabolite may be indicative of increased oxidative stress in the post operative patients.

Four out of the 16 identified differential metabolites, succinate, phenylacetylglutamine, 2-hydroxyhippurate, and 5-hydroxytryptophan showed recovering tendency in the postoperative samples compared with the preoperative samples in their mean values. Such an alteration is due to several factors. For example, colon flush prior to surgery removed most of the gut flora and as a result, all gut flora related metabolites, including phenylacetylglutamine and 2-hydroxyhippurate, are down-regulated. The increased level of succinate is presumably derived from the amino acids supplementation, as discussed in the above paragraph. The significantly down regulated 5-hydrotryptophan in post-operative subjects may suggest an improved tryptophan metabolism as a result of surgery.

This investigation identified clear distinctions were observed for both cancerous human subjects and precancerous colorectal lesion rats as compared to their healthy counterparts. These results demonstrate the potential of this non-invasive urinary metabonomic strategy as a complementary diagnostic tool for CRC. Several important metabolic pathways disturbed in association with CRC morbidity, most of which were shared in the human and rat samples. Notably, tryptophan metabolism is highlighted in several observations including serum metabonomic study, urinary metabonomic results of rat model, preoperative, and post operative urine samples.

### Example 3

### Metabolite Markers for Colorectal Cancer Identified in Serum (Expanded Study)

### Material and Methods for Serum Analysis

Chemicals. HPLC grade methanol and chloroform were obtained from Merck Chemicals (Darmstadt, Germany). Analytical grade pyridine, and anhydrous sodium sulfate were Analytical grade from China National Pharmaceutical Group Corporation (Shanghai, China). L-2-chlorophenylalanine was Analytical grade from Intechem Tech. Co. Ltd. (Shanghai, China). BSTFA (1% TMCS), heptadecanoic acid and methoxyamine were purchased from Sigma-Aldrich (St. Louis, Mo., USA).

Clinical samples. A total of 103 CRC patients between 24 to 83 years were enrolled in the study. The patients were newly diagnosed with CRC and had not received any medication. According to TNM classification of malignant tumors based on histopathological features, the patients were classified into CRC stages: stage I (26 patients), stage II (45 patients), stage III (27 patients), and stage IV (5 patients). The clinical diagnosis and pathological reports of all the patients were obtained from the hospital (Ruijin hospital, Shanhai, China). The healthy volunteers (106 individuals), ages 31 to 76 years, were selected by a routine physical examination and any subjects with inflammatory conditions or gastrointestinal tract disorders were excluded. Clinical information on participants is provided in Table 11. Venous blood was collected in the morning before breakfast from a total of 103 CRC patients and 106 healthy volunteers at Ruijin hospital (Shanghai, China). The protocol was approved by the Ruijin Hospital Institutional Review Board and all participants gave informed consent before they were involved in the study. These procedures are consistent with those set forth in Example

TABLE 11
Demographic and Clinical Chemistry
Characteristics of Human Subjects

| | CRC patients | Healthy controls |
|---|---|---|
| n | 103 | 106 |
| Age (median, range) | 60, 24-83 | 58, 31-76 |
| Male/female ratio | 69/44 | 31/75 |
| Stage I | 26 | / |
| Stage II | 45 | / |
| Stage III | 27 | / |
| Stage IV | 5 | / |

GC-TOFMS spectral acquisition of serum samples and data pretreatment. A 100 μL aliquot of serum sample was spiked with two internal standard solutions (10 μl L-2-chlorophenylalanine in water, 0.3 mg/ml; 10 μL heptadecanoic acid in methanol, 1 mg/mL) and vortexed for 10 seconds. The mixed solution was extracted with 300 μL of pre-cooled (−20° C.) methanol:chloroform (3:1) and vortexed for 30 seconds. After storing for 10 minutes at −20° C., the samples were centrifuged at 10,000 g for 10 minutes at 4° C. An aliquot of the 300 μL supernatant was transferred to a glass sampling vial to vacuum dry at room temperature. The residue was derivatized using a two-step procedure. First, 80 μL methoxyamine (15 mg/mL in pyridine) was added to the vial and kept at 30° C. for 90 minutes. An amount of 10 μL retention index compounds (the mixture of 010-040, 50 μg/mL), and 80 μL BSTFA (1% TMCS) were added into the reaction vials. Then the samples were subjected to a 70° C. for 60 minutes derivatization reaction.

Each 1-μL aliquot of the derivatized solution was injected in splitless mode into an Agilent 7890N gas chromatograph coupled with a Pegasus HT time-of-flight mass spectrometer (Leco Corporation, St Joseph, USA). Separation was achieved on a DB-5 ms capillary column (30 m×250 μm I.D., 0.25-μm film thickness; Agilent J&W Scientific, Folsom, Calif., USA), with helium as the carrier gas at a constant flow rate of 1.0 ml/min. The temperature of injection, transfer interface, and ion source was set to 260° C., 260° C., and 210° C., respectively. The GC temperature programming was set to 2 min isothermal heating at 80° C., followed by 10° C./min oven temperature ramps to 220° C., 5° C./min to 240° C., and 25° C./min to 290° C., and a final 8 min maintenance at 290° C. Electron impact ionization (70 eV) at full scan mode (m/z 40-600) was used, with an acquisition rate of 20 spectra/second in the TOFMS setting. These procedures are consistent with those set forth in Example 1.

GC-TOFMS Data Analysis. The acquired MS files from GC/TOFMS analysis were analyzed by ChromaTOF software (v4.22, Leco Co., CA, USA). After alignment by the Statistics component in ChromaTOF software, a three dimensional data set includes sample information, peak retention time and peak intensities was obtained. Internal standards and any known artificial peaks, such as peaks caused by noise, column bleed and BSTFA derivatization procedure, were removed from the data set. The resulting data was normalized to internal standard prior to statistic analysis. The normalized data was mean centered and unit variance scaled during chemometric data analysis in the SIMCA-p 12.0 Software package (Umetrics, Umeå, Sweden). Differential variables were selected based on the VIP values (VIP>1) in the OPLS-DA model and the Wilcoxon-Mann-Whitney test (p<0.05). The Wilcoxon-Mann-Whitney test was performed in SPSS software (19.0, IBM, USA). Differential variables were selected based on the Student's t test (p<0.05). Box-plots were also performed in SPSS software. Compound identification was performed by comparing the mass fragments with NIST 05 Standard mass spectral databases in NIST MS search 2.0 (NIST, Gaithersburg, Md.) software with a similarity of more than 70% and verified by available reference compounds. These procedures are consistent with those set forth in Example 1.

### Results

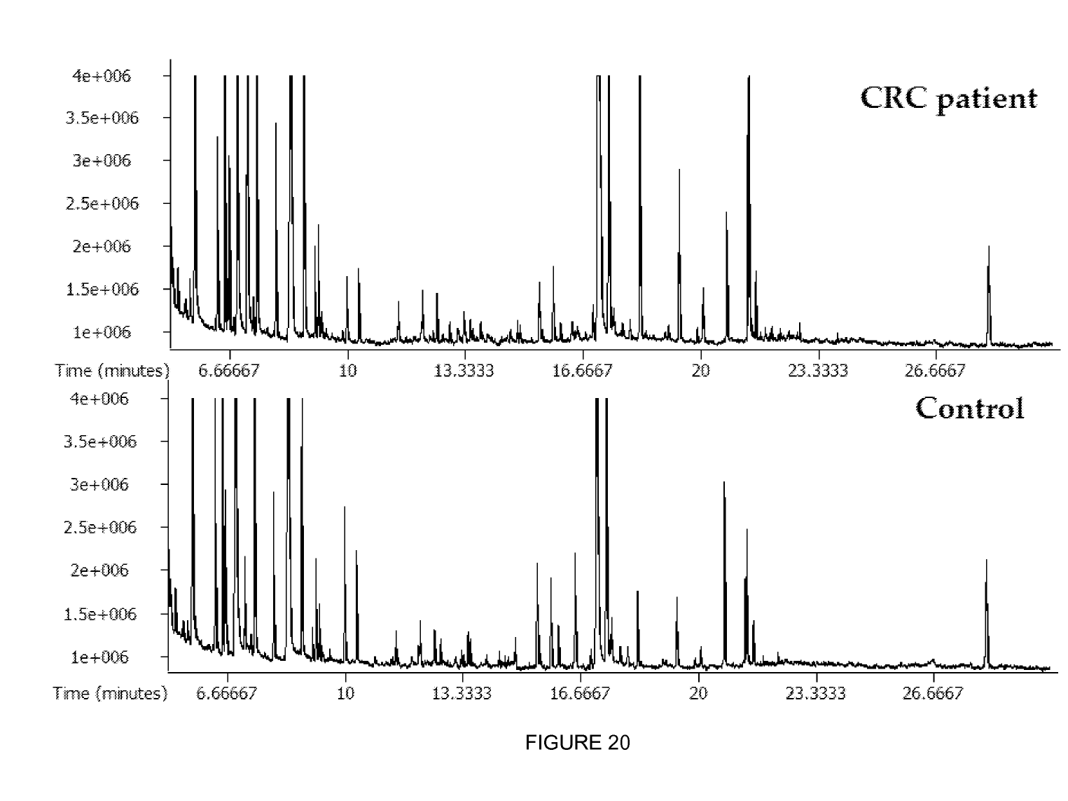

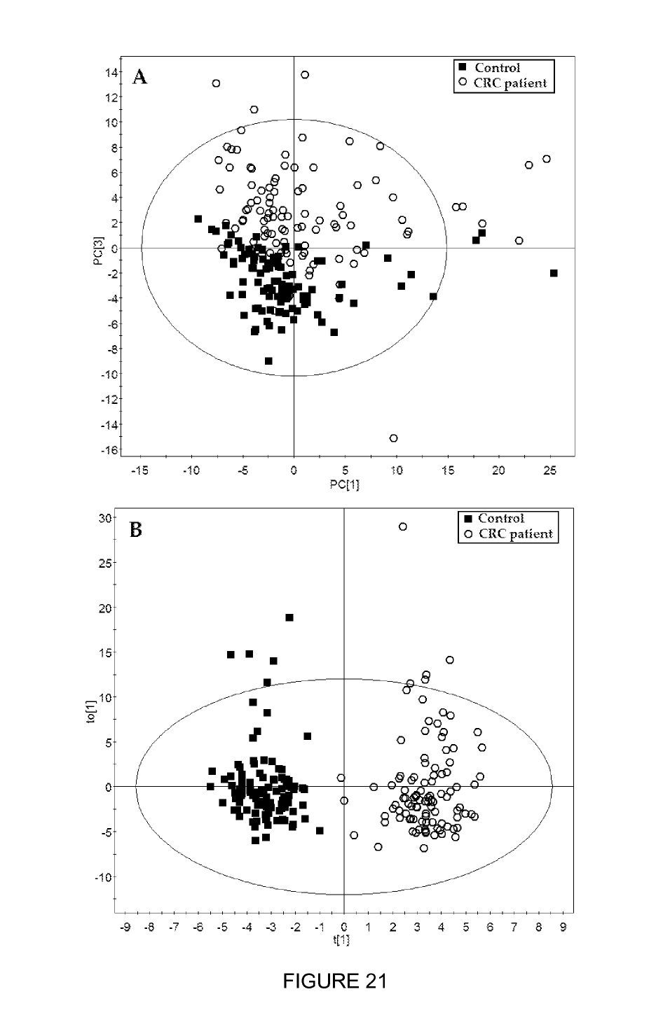

GC-TOFMS analysis. Typical GC-TOFMS total ion current (TIC) chromatograms of serum samples from one cancer patient and one healthy control are shown in FIG. 20. The spectrum was analyzed with statistic component function in ChormaTOF software. After removal of two internal standards, a total of 219 variables were obtained and used in the following analysis. The data was normalized to one of the internal standards (L-2-chlorophenylalanine, with lower relative standard derivation than heptadecanoic acid among all the samples). The normalized data was input into SIMCA-p software (v12.0). PCA was first performed. After removal of four outliers (these samples were excluded in all of the flowing analysis), a separation tendency can be seen from the scores plot (FIG. 21A, PC1 vs PC 3, 9 components, R2X=0.584, Q2=0.396). Clear separation was obtained from the OPLS-DA model as shown in FIG. 21B (one prediction component with 4 orthogonal components, R2X=0.437, R2Y=0.916, Q2=0.853). These procedures are consistent with those set forth in Example 1.

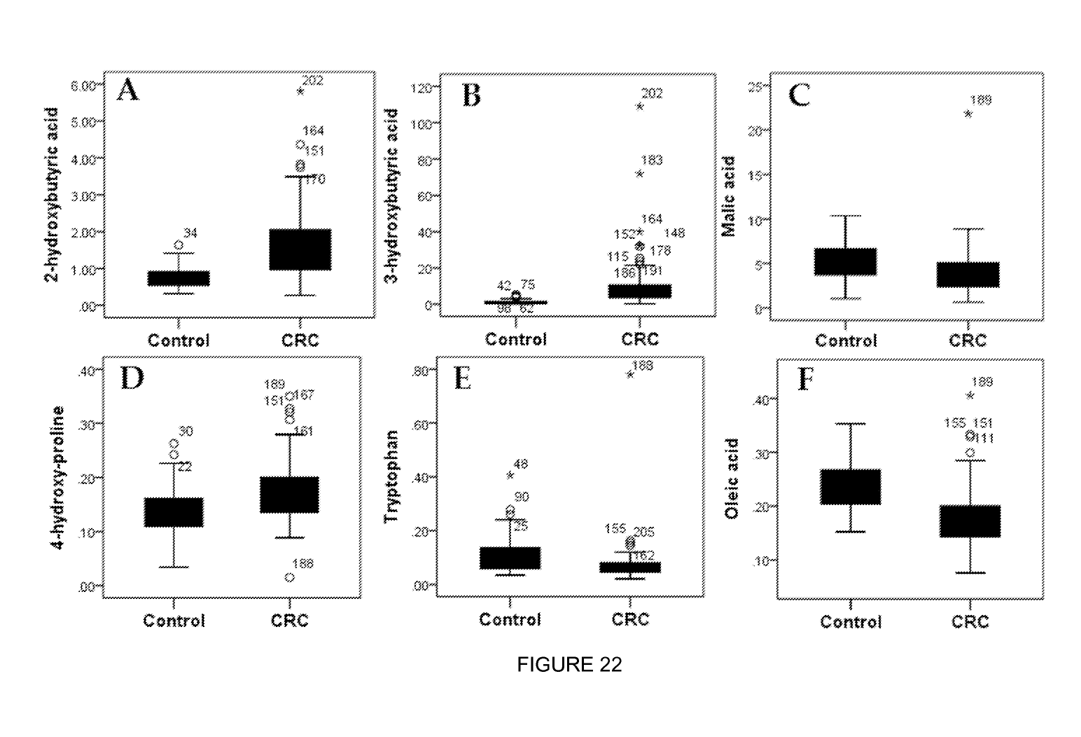

Wilcoxon-Mann-Whitney test statistical analysis. Based on VIP values from OPLS-DA model and the Wilcoxon-Mann-Whitney test, 59 differential variables were obtained. A number of 43 metabolites were identified from those 59 differential variables, and 35 were verified by authentic standards (Table 12). The different expression levels in the serum of individual metabolites were shown in the box-plots of six typical metabolites (FIG. 22). This analysis is consistent with that set forth in Example 1.

TABLE 12
Differential metabolites derived from OPLS-DA mode of GC-TOFMS analysis
with Wilcoxon-Mann-Whitney test

| | | Retention | | | | |
|---|---|---|---|---|---|---|
| No. | Metabolite^{a} | Time (min) | VIP^{C} | P^{d} | FC^{e} | Chemical Class |
| 1 | Pyruvic acid^{b} | 5.56 | 1.03 | 1.48E−03 | 1.29 | Organic |
| | | | | | | acid |
| 2 | Alanine^{b} | 6.33 | 1.76 | 1.02E−09 | −1.65 | Amino acid |
| 3 | 2-hydroxybutyric acid^{b} | 6.66 | 2.45 | 2.25E−19 | 2.13 | Organic |
| | | | | | | acid |
| 4 | 2-oxo-butanoic acid | 6.79 | 2.33 | 1.20E−26 | 2.49 | Organic |
| | | | | | | acid |
| 5 | p-Cresol^{b} | 7.13 | 1.14 | 6.31E−05 | 1.38 | Aromatic |
| | | | | | | compound |
| 6 | 3-hydroxybutyric acid^{b} | 7.19 | 1.83 | 3.79E−25 | 2.41 | Organic |
| | | | | | | acid |
| 7 | Octanoic acid^{b} | 7.27 | 1.01 | 1.12E−03 | 1.30 | Organic |
| | | | | | | acid |
| 8 | 2-aminobutanoic acid^{b} | 7.36 | 2.43 | 4.76E−16 | 1.96 | Amino acid |
| 9 | Urea^{b} | 7.45 | 1.29 | 2.29E−07 | −1.53 | Amine |
| 10 | Glycerol^{b} | 8.78 | 2.39 | 3.14E−18 | 2.07 | Lipid |
| 11 | Allisoleucine | 9.03 | 1.83 | 2.95E−09 | 1.62 | Amino acid |
| 12 | Isoleucine^{b} | 9.12 | 1.18 | 3.44E−04 | 1.34 | Amino acid |
| 13 | Succinic acid^{b} | 9.43 | 1.02 | 4.63E−02 | 1.17 | Organic |
| | | | | | | acid |
| 14 | Glyceric acid^{b} | 9.57 | 2.48 | 8.83E−16 | −1.96 | Organic |
| | | | | | | acid |
| 15 | Methylmaleic acid | 9.87 | 1.52 | 9.81E−13 | 1.80 | Organic |
| | | | | | | acid |
| 16 | Fumarate^{b} | 9.91 | 2.61 | 8.60E−23 | −2.32 | Organic |
| | | | | | | acid |
| 17 | Serine^{b} | 10.00 | 1.10 | 4.34E−04 | −1.33 | Amino acid |
| 18 | Aminomalonic acid^{b} | 11.46 | 1.03 | 9.53E−05 | −1.37 | Amino acid |
| 19 | Malic acid^{b} | 11.69 | 1.52 | 2.91E−06 | 1.46 | Organic |
| | | | | | | acid |
| 20 | Threitol^{b} | 11.89 | 1.69 | 2.18E−12 | −1.79 | Polyol |
| 21 | Aspartic acid^{b} | 12.08 | 2.41 | 1.36E−16 | −2.00 | Amino acid |
| 22 | 4-hydroxy-proline^{b} | 12.16 | 1.78 | 5.40E−08 | −1.56 | Amino acid |
| 23 | à-Aminoadipic acid | 12.43 | 1.26 | 4.08E−04 | −1.33 | Amino acid |
| 24 | Creatinine^{b} | 12.54 | 1.03 | 3.17E−09 | 1.62 | Amine |
| 25 | 2-oxo-pentanedioic | 12.81 | 1.22 | 1.41E−05 | 1.42 | Organic |
| | acid^{b} | | | | | acid |
| 26 | Phenylalanine^{b} | 13.50 | 2.06 | 2.01E−15 | −1.95 | Amino acid |
| 27 | Asparagine^{b} | 14.02 | 1.17 | 9.72E−05 | −1.37 | Amino acid |
| 28 | Ribitol | 14.44 | 1.90 | 2.10E−10 | −1.69 | Polyol |
| 29 | Hypoxanthine^{b} | 15.93 | 1.07 | 3.29E−04 | 1.34 | Organic |
| | | | | | | acid |
| 30 | d- | 16.51 | 1.40 | 2.91E−06 | −1.47 | Amino acid |
| | Erythrotetrofuranose | | | | | |
| 31 | Histidine^{b} | 17.51 | 1.70 | 2.20E−08 | −1.58 | Amino acid |
| 32 | Palmitic acid^{b} | 19.41 | 1.06 | 6.12E−05 | 1.38 | Fatty acid |
| 33 | Gluconic acid, | 21.28 | 2.07 | 5.03E−12 | −1.77 | Sugar |
| | phosphate | | | | | phosphate |
| 34 | Tryptophan^{b} | 21.32 | 2.48 | 2.83E−18 | −2.09 | Amino acid |
| 35 | Linolic acid^{b} | 21.33 | 1.33 | 1.28E−05 | 1.42 | Fatty acid |
| 36 | Oleic acid^{b} | 21.38 | 1.55 | 1.33E−08 | 1.59 | Fatty acid |
| 37 | 9-octadecenoate^{b} | 21.42 | 1.27 | 7.41E−06 | 1.44 | Fatty acid |
| 38 | Octadecanoic acid^{b} | 21.56 | 1.14 | 1.08E−05 | 1.43 | Fatty acid |
| 39 | Cystine^{b} | 21.83 | 1.68 | 1.49E−07 | 1.53 | Amino acid |
| 40 | 11,14-Eicosadienoic | 22.50 | 1.21 | 3.69E−03 | 1.26 | Fatty acid |
| | acid^{b} | | | | | |
| 41 | Oleamide^{b} | 22.50 | 2.20 | 3.17E−10 | 1.67 | Fatty acid |
| | | | | | | amine |
| 42 | Matitol | 23.87 | 3.56 | 7.11E−29 | 2.61 | Sugar |
| 43 | Cholesterol^{b} | 28.17 | 1.03 | 7.25E−06 | −1.44 | Cholesterol |

^{a}Metabolites are identified using available library databases;
^{b}verified by reference compounds.
^{c}Variable importance in the projection (VIP) was obtained from OPLS-DA with a threshold of 1.0.
^{d}P-value and fold change (FC) are calculated from nonparametric Wilcoxon-Mann-Whitney test.
^{e}FC with a positive value indicates a relatively higher concentration present in CRC patients while a negative value means a relatively lower concentration as compared to the healthy controls.

Student's t test statistical analysis. Based on p values from Student's t test (p<0.05), 96 differential variables were obtained. A number of 64 metabolites were identified from those 96 differential variables, and 45 were verified by comparison to commercially obtained reference standards (Table 13). This analysis is consistent with that set forth in Example 1.

TABLE 13
Differential metabolites between CRC patients
and controls using Student's t test (p < 0.05)

| | | Retention | | | |
|---|---|---|---|---|---|
| | | Time | | | Chemical |
| No. | Metabolite^{a} | (min) | P^{c} | FC^{d} | Class |
| 1 | Pyruvic acid^{b} | 5.56 | 1.31E−03 | 1.23 | Organic acid |
| 2 | Ethanolamine | 5.59 | 2.39E−02 | 1.11 | Amine |
| 3 | Alanine^{b} | 6.33 | 5.42E−09 | −1.31 | Amino acid |
| 4 | 2-hydroxybutyric | 6.66 | 5.41E−18 | 2.22 | Organic acid |
| | acid^{b} | | | | |
| 5 | 2-oxo-butanoic acid | 6.79 | 2.98E−16 | 3.92 | Organic acid |
| 6 | p-cresol^{b} | 7.13 | 2.04E−04 | 1.15 | Aromatic |
| | | | | | compound |
| 7 | 3-hydroxybutyric | 7.19 | 7.66E−10 | 9.38 | Organic acid |
| | acid^{b} | | | | |
| 8 | 2-hydroxy-3- | 7.22 | 4.68E−02 | 1.21 | Organic acid |
| | methylbutric acid | | | | |
| 9 | Octanoic acid^{b} | 7.27 | 1.11E−03 | 1.20 | Organic acid |
| 10 | 2-aminobutanoic | 7.36 | 2.54E−17 | 1.74 | Amino acid |
| | acid^{b} | | | | |
| 11 | 2-oxo-4- | 7.74 | 1.20E−03 | 1.56 | Organic acid |
| | methylvaleric acid | | | | |
| 12 | Urea^{b} | 8.49 | 4.34E−03 | −1.26 | Amine |
| 13 | Benzoic acid | 8.52 | 2.53E−03 | 1.26 | Aromatic |
| | | | | | compound |
| 14 | Glycerol^{b} | 8.78 | 9.62E−17 | 2.67 | Lipid |
| 15 | Allisoleucine | 9.03 | 6.80E−10 | 1.44 | Amino acid |
| 16 | Isoleucine^{b} | 9.12 | 4.57E−05 | 1.20 | Amino acid |
| 17 | Succinic acid^{b} | 9.43 | 5.33E−04 | 3.39 | Organic acid |
| 18 | Glyceric acid^{b} | 9.57 | 5.02E−18 | −1.47 | Organic acid |
| 19 | Methylmaleic acid | 9.87 | 4.79E−07 | 1.80 | Organic acid |
| 20 | Fumarate^{b} | 9.91 | 2.71E−20 | −2.06 | Organic acid |
| 21 | Serine^{b} | 10.00 | 3.49E−04 | −1.11 | Amino acid |
| 22 | 2- | 10.13 | 1.53E−03 | −1.42 | Organic acid |
| | Piperidinecarboxylic | | | | |
| | acid | | | | |
| 23 | Threonine^{b} | 10.34 | 3.57E−02 | −1.08 | Amino acid |
| 24 | Pyrrole-2-carboxylic | 10.34 | 4.60E−03 | 1.22 | Organic acid |
| | acid | | | | |
| 25 | Beta-alanine^{b} | 10.93 | 2.26E−02 | −1.19 | Amino acid |
| 26 | Aminomalonic acid^{b} | 11.46 | 8.82E−04 | −1.39 | Amino acid |
| 27 | Malic acid^{b} | 11.69 | 2.55E−06 | 1.24 | Organic acid |
| 28 | Threitol^{b} | 11.89 | 1.79E−08 | −1.23 | Polyol |
| 29 | Aspartic acid^{b} | 12.08 | 6.34E−17 | −1.54 | Amino acid |
| 30 | Methionine^{b} | 12.09 | 3.04E−03 | −1.11 | Amino acid |
| 31 | 5-oxoproline^{b} | 12.14 | 1.03E−02 | −1.14 | Amino acid |
| 32 | 4-hydroxy-proline^{b} | 12.16 | 3.04E−04 | −1.49 | Amino acid |
| 33 | a-Aminoadipic acid | 12.43 | 4.16E−05 | −1.68 | Amino acid |
| 34 | Creatinine^{b} | 12.54 | 5.36E−04 | 1.49 | Amine |
| 35 | 2-oxo-pentanedioic | 12.81 | 4.52E−05 | 1.22 | Organic acid |
| | acid^{b} | | | | |
| 36 | Phenylalanine^{b} | 13.50 | 2.87E−12 | −1.27 | Amino acid |
| 37 | Xylose^{b} | 13.86 | 3.75E−03 | 1.13 | Sugar |
| 38 | Asparagine^{b} | 14.02 | 1.68E−04 | −1.14 | Amino acid |
| 39 | Arabinose | 14.05 | 2.70E−03 | −1.36 | Sugar |
| 40 | Ribitol | 14.44 | 5.62E−08 | −1.35 | Polyol |
| 41 | Rhamnose | 14.71 | 2.56E−02 | 2.27 | Sugar |
| 42 | Hypoxanthine^{b} | 15.45 | 5.49E−03 | −1.09 | Organic acid |
| 43 | Glutamine^{b} | 15.93 | 3.47E−04 | 1.24 | Amino acid |
| 44 | Ornithine^{b} | 16.07 | 2.02E−03 | −1.18 | Amino acid |
| 45 | Erythrotetrofuranose | 16.51 | 5.71E−06 | −1.28 | Amino acid |
| 46 | Myristic acid^{b} | 16.58 | 3.35E−02 | 1.18 | Fatty acid |
| 47 | Fructose | 16.77 | 4.15E−02 | −1.18 | Sugar |
| 48 | Histidine^{b} | 17.51 | 2.41E−08 | −1.20 | Amino acid |
| 49 | Tyrosine^{b} | 17.80 | 4.43E−03 | 1.10 | Amino acid |
| 50 | Gluconic acid | 19.00 | 2.41E−03 | −1.11 | Sugar |
| | | | | | derivate |
| 51 | Palmitic acid^{b} | 19.41 | 9.60E−04 | 1.21 | Fatty acid |
| 52 | Myo-Inositol | 19.92 | 1.84E−02 | −1.12 | Polyol |
| 53 | Gluconic acid, | 21.28 | 2.00E−12 | −1.41 | Sugar |
| | phosphate | | | | phosphate |
| 54 | Tryptophan^{b} | 21.32 | 6.34E−18 | −1.38 | Amino acid |
| 55 | Linolic acid^{b} | 21.33 | 2.17E−05 | 1.31 | Fatty acid |
| 56 | Oleic acid^{b} | 21.38 | 3.17E−07 | 1.49 | Fatty acid |
| 57 | 9-octadecenoate^{b} | 21.42 | 4.46E−05 | 1.37 | Fatty acid |
| 58 | Octadecanoic acid^{b} | 21.56 | 4.85E−04 | 1.16 | Fatty acid |
| 59 | Cystine^{b} | 21.83 | 2.68E−07 | 1.19 | Amino acid |
| 60 | 11,14-Eicosadienoic | 22.50 | 1.18E−04 | 1.40 | Fatty acid |
| | acid^{b} | | | | |
| 61 | Oleamide^{b} | 22.50 | 2.73E−13 | 3.11 | Fatty acid |
| | | | | | amine |
| 62 | Matitol | 23.87 | 8.14E−47 | 12.15 | Sugar |
| 63 | Vitamin E^{b} | 27.93 | 1.48E−02 | −1.15 | Vitamine |
| 64 | Cholesterol^{b} | 28.17 | 5.47E−04 | −1.15 | Cholesterol |

^{a}Metabolites are identified using available library databases;
^{b}verified by reference compounds.
^{c}P-values are calculated from Student's t test.
^{d}Fold changes (FC) are calculated from mean values. FC with a positive value indicates a relatively higher concentration present in CRC patients while a negative value means a relatively lower concentration as compared to the healthy controls.

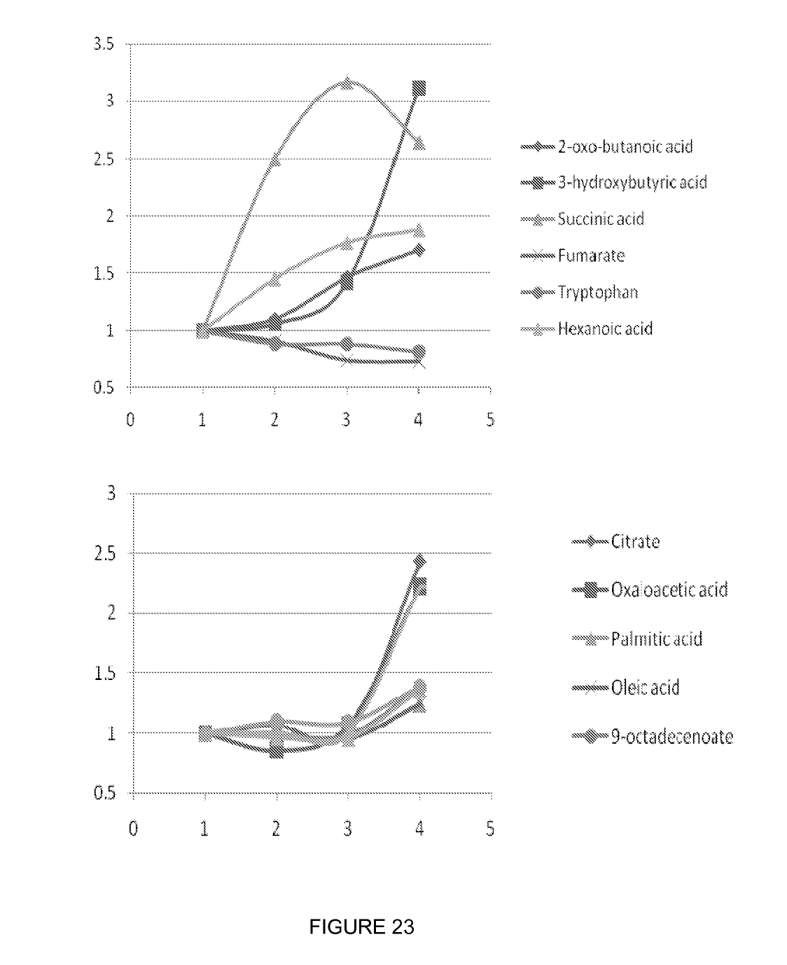

CRC Stage Differentiation of Markers. Some metabolites showed some characteristic in certain pathological stage (TNM stage), which may be the biomarkers for stage discrimination (Table 14 and FIG. 23). The values were normalized to the corresponding mean values from stage I patients. Interestingly, some fatty acids such as oleic acid, palmitic acid, and myristic acid, and citrate and oxaloacetic acid showed a consistent increase tendency in stage IV patients, which may reveal higher fatty acid synthesis from citrate in stage IV patients. In addition, 3-hydroxybutryic acid also increased dramatically in stage IV patients compared with that in other patients. These results may reveal higher activity of fatty acid metabolism in metastatic CRC patients.

TABLE 14
Some metabolite alteration in different CRC stages.

| | Stage 1 | | Stage 2 | | Stage 3 | | Stage 4 | |
|---|---|---|---|---|---|---|---|---|
| Metabolite | mean | SEM^{a} | mean | SEM | mean | SEM | mean | SEM |
| 2-oxo-butanoic | 1 | 0.62 | 1.10 | 0.79 | 1.47 | 1.36 | 1.70 | 1.96 |
| acid | | | | | | | | |
| 3- | 1 | 1.04 | 1.06 | 1.05 | 1.42 | 1.71 | 3.11 | 5.81 |
| hydroxybutyric | | | | | | | | |
| acid | | | | | | | | |
| Succinic acid | 1 | 1.23 | 2.50 | 5.32 | 3.16 | 5.83 | 2.64 | 2.69 |
| Fumarate | 1 | 0.63 | 0.91 | 0.84 | 0.74 | 0.41 | 0.73 | 0.27 |
| Tryptophan | 1 | 0.28 | 0.89 | 0.28 | 0.88 | 0.32 | 0.81 | 0.17 |
| Hexanoic acid | 1 | 1.00 | 1.45 | 2.40 | 1.77 | 1.61 | 1.88 | 2.15 |
| Citrate | 1 | 0.24 | 1.07 | 0.42 | 1.01 | 0.59 | 2.44 | 3.36 |
| Oxaloacetic | 1 | 0.88 | 0.86 | 0.97 | 1.07 | 1.30 | 2.22 | 3.55 |
| acid | | | | | | | | |
| Palmitic acid | 1 | 0.35 | 1.00 | 0.33 | 0.96 | 0.37 | 1.24 | 1.19 |
| Oleic acid | 1 | 0.53 | 0.99 | 0.40 | 0.99 | 0.44 | 1.38 | 1.65 |
| 9- | 1 | 0.47 | 1.04 | 0.43 | 1.02 | 0.47 | 1.52 | 1.72 |
| octadecenoate | | | | | | | | |
| 11,14- | 1 | 0.49 | 1.10 | 0.78 | 1.09 | 0.84 | 1.39 | 0.71 |
| Eicosadienoic | | | | | | | | |
| acid | | | | | | | | |
| Myristic acid | 1 | 0.49 | 0.97 | 0.44 | 0.96 | 0.48 | 1.36 | 1.43 |

^{a}Standard error of the mean.

### Conclusions

Thus, according to an embodiment of the invention, a GC-TOFMS-based serum metabolomics approach enabled identification of a number of metabolites that are differentially present in subjects with CRC as compared to healthy subjects (Table 15). Thus, in some aspects of the invention, GC-TOFMS can be used to detect metabolites as biomarkers for the diagnosis and/or prognosis of subjects with CRC. The identified potential biomarkers showed lower levels of some sugars and polyols such as threitol, ribitol, and eyrthrotetrofuranose. In addition, some fatty acids and their product, 3-hydroxybyric acid, were detected at higher levels in the CRC patients. These results may indicate higher energy consumption in the CRC patients. Some amino acids (such as tryptophan, asparagine, aspartic acid, and alanine) were detected at lower levels in the serum of CRC patients, while 2-aminobutyric acid was observed at higher levels in CRC patients. The higher level of 2-aminobutyrioc acid may indicate higher oxidative stress in the CRC patients. The disordered levels of TCA intermediates, such as e.g., lower levels of fumarate and 2-oxopentanedioic acid and higher levels of succinic acid, may indicate disrupted mitochondrial function in the CRC patients. Other differential metabolites were identified as well.

TABLE 15
Differential serum metabolites for CRC

| | | Retention | | | | |
|---|---|---|---|---|---|---|
| No. | Metabolite^{a} | Time (min) | VIP^{C} | P^{d} | FC^{e} | Chemical Class |
| 1 | Pyruvic acid^{b} | 5.56 | 1.03 | 1.48E−03 | 1.29 | Organic acid |
| 2 | 2-hydroxybutyric acid^{b} | 6.66 | 2.45 | 2.25E−19 | 2.13 | Organic acid |
| 3 | p-cresol^{b} | 7.13 | 1.14 | 6.31E−05 | 1.38 | Aromatic |
| | | | | | | compound |
| 4 | 3-hydroxybutyric | 7.19 | 1.83 | 3.79E−25 | 2.41 | Organic acid |
| | acid^{b} | | | | | |
| 5 | 2-aminobutanoic | 7.36 | 2.43 | 4.76E−16 | 1.96 | Amino acid |
| | acid^{b} | | | | | |
| 6 | Urea^{b} | 7.45 | 1.29 | 2.29E−07 | −1.53 | Amine |
| 7 | Isoleucine^{b} | 9.12 | 1.18 | 3.44E−04 | 1.34 | Amino acid |
| 8 | Succinic acid^{b} | 9.43 | 1.02 | 4.63E−02 | 1.17 | Organic acid |
| 9 | Fumarate^{b} | 9.91 | 2.61 | 8.60E−23 | −2.32 | Organic acid |
| 10 | Methylmaleic | 9.87 | 1.52 | 9.81E−13 | 1.80 | Organic acid |
| | acid | | | | | |
| 11 | Malic acid^{b} | 11.69 | 1.52 | 2.91E−06 | 1.46 | Organic acid |
| 12 | Aspartic acid^{b} | 12.08 | 2.41 | 1.36E−16 | −2.00 | Amino acid |
| 13 | 4-hydroxy-proline^{b} | 12.16 | 1.78 | 5.40E−08 | −1.56 | Amino acid |
| 14 | 2-oxo- | 12.81 | 1.22 | 1.41E−05 | 1.42 | Organic acid |
| | pentanedioic | | | | | |
| | acid^{b} | | | | | |
| 15 | Phenylalanine^{b} | 13.50 | 2.06 | 2.01E−15 | −1.95 | Amino acid |
| 16 | Asparagine^{b} | 14.02 | 1.17 | 9.72E−05 | −1.37 | Amino acid |
| 17 | Hypoxanthine^{b} | 15.93 | 1.07 | 3.29E−04 | 1.34 | Nuclear acid |
| 18 | Histidine^{b} | 17.51 | 1.70 | 2.20E−08 | −1.58 | Amino acid |
| 19 | Palmitic acid^{b} | 19.41 | 1.06 | 6.12E−05 | 1.38 | Fatty acid |
| 20 | Tryptophan^{b} | 21.32 | 2.48 | 2.83E−18 | −2.09 | Amino acid |
| 21 | Linolic acid^{b} | 21.33 | 1.33 | 1.28E−05 | 1.42 | Fatty acid |
| 22 | Oleic acid^{b} | 21.38 | 1.55 | 1.33E−08 | 1.59 | Fatty acid |
| 23 | Octadecanoic acid^{b} | 21.56 | 1.14 | 1.08E−05 | 1.43 | Fatty acid |
| 24 | Oleamide^{b} | 22.50 | 2.20 | 3.17E−10 | 1.67 | Fatty acid |
| | | | | | | amine |

^{a}Metabolites are identified using available library databases;
^{b}verified by reference compounds.
^{c}Variable importance in the projection (VIP) was obtained from OPLS-DA with a threshold of 1.0.
^{d}P-value and fold change (FC) are calculated from nonparametric Wilcoxon-Mann-Whitney test.
^{e}FC with a positive value indicates a relatively higher concentration present in CRC patients while a negative value means a relatively lower concentration as compared to the healthy controls.

### Example 4

### Metabolite Markers for Colorectal Cancer Identified in Urine (Expanded Study)

### Material and Methods for Urine Analysis

Chemicals. HPLC grade methanol was obtained from Merck Chemicals (Darmstadt, Germany). Analytical grade pyridine and anhydrous sodium sulfate were obtained from China National Pharmaceutical Group Corporation (Shanghai, China). L-2-chlorophenylalanine was obtained from Intechem Tech. Co. Ltd. (Shanghai, China). Urease, BSTFA (1% TMCS), heptadecanoic acid and methoxyamine were obtained from Sigma-Aldrich (St. Louis, Mo., USA).

Clinical samples. Urine samples were collected from the same CRC patients and volunteers as described in Example 3. Samples were collected and processed in accordance with the procedures set forth in Example 2.

GC-MS spectral acquisition of urine samples. For each 100 μl urine sample, a total of 10 μl of urease (30 U/10 μl, Type C) was added and the sample was stored at 37° C. for 15 min. Two internal standard solutions (10 μl of L-2-chlorophenylalanine in water, 0.1 mg/ml; 10 μl of heptadecanoic acid in methanol, 1 mg/ml) were added to the sample. An aliquot of 300 μl of methanol was added for metabolite extraction. After centrifugation at 16,100×g at 4° C. for 5 min, an aliquot of 300 μl of the supernatant was transferred to a GC sampling vial for vacuum drying at room temperature. The residue remaining after drying off the supernatant was derivatized with 80 μl of methoxyamine HCl (15 mg/ml in pyridine) at 30° C. for 90 minutes and followed by 80 μl BSTFA (1% TMCS) at 70° C. for 60 minutes.

Each 1 μL aliquot of the derivatized solution was injected in splitless mode into a GC-TOF mass spectrometer (Leco Corporation, St. Joseph, USA). A DB-5 ms capillary column (30 m×250 μm I.D., 0.25-μm film thickness; Agilent J&W Scientific, Folsom, Calif., USA) was also used to assess the urine samples using helium as the carrier gas at a constant flow rate of 1.0 ml/min. The temperature of injection, transfer interface, and ion source was set to 260° C., 260° C. and 210° C., respectively. The GC temperature programming was set to 2 min isothermal heating at 80° C., followed by 10° C./min oven temperature ramps to 140° C., 4° C./min to 210° C., 10° C./min to 240° C., and 25° C./min to 290° C., and a final 4.5 min maintenance at 290° C. Electron impact ionization (70 eV) at full scan mode (m/z 40-600) was used, with an acquisition rate of 20 spectra/second in the TOFMS setting. These procedures are consistent with those set forth in Example 2.

UPLC-QTOFMS analysis of urine samples. The urine samples were allowed to thaw at room temperature, vortexed for 1 min, and centrifuged at 13,000 rpm (15,700 g) for 5 min prior to analysis. A 300 μL of supernatant was diluted with 600 μL of ultrapure water containing internal standard of L-2-chlorophenylalanine (10 μg/mL), vortexed and filtered through a PTFE syringe filter (0.22 μm) for analysis (1, 2).

Chromatographic separations were performed on a 2.1×100 mm 1.7 μm ACQUITY BEH C18 column (Waters Corp., Milford, Mass.) using an ultra performance liquid chromatography system (Waters Corp., Milford, Mass.). The column was maintained at 40° C. and the temperature of the system's sampler manager was maintained at 4° C. Mobile phases consisting of water+0.1% formic acid (Solution A) and acetonitrile+0.1% formic acid (Solution B) were set as gradient elution. Elution started with 1% of Solution B for 1 min, increasing to 100% of Solution B over 8.5 min, maintaining 100% of Solution B for 1 min, re-equilibrating to the initial condition (1% of Solution B) in 1 min and maintaining for 1.5 min. The flow rate was 0.4 mL/min and the injection volume was 5 μL.

A Waters Q-TOF Premier MS system (Waters Corp., Milford, Mass.) was operated in positive ion electrospray ionization (ESI) mode. The temperature and flow rate for desolvation gas (nitrogen) was set to 350° C. and 600 L/h, respectively. The cone gas (nitrogen) was set to 50 L/h and the source temperature was 100° C. The capillary and cone voltages were set to 3, 200 and 35 V, respectively. The centroid data was acquired for each sample from 50 to 1,000 Da with a 0.28 s scan time and a 0.02 s interscan delay over a 13 min run time. Leucine-enkephalin (m/z 556.2771 in positive mode) was used as external lockmass at a concentration of 200 pg/μL with the flow rate of 0.1 mL/min and introduced to LockSpray interface to perform online exact mass correction. These procedures are consistent with those set forth in Example 1.

Data Analysis. For UPLC-QTOFMS data, the acquired raw data files were processed by the MarkerLynx application manager version 4.1 (Waters Corp., Milford, Mass.) for peak detection and alignment (1, 2). The parameters were set as: retention time (RT) range 0-9 min, mass range 50-1000 Da and mass tolerance 0.02 Da; internal standard detection parameters were deselected for peak retention time alignment; isotopic peaks and background ions from associated blank injections were excluded for analysis; noise elimination level was set at 10; minimum intensity was set to 15% of base peak intensity; maximum masses per RT were set at 6, RT tolerance was set at 0.01 min; peak width at 5% height and peak-to-peak baseline noise was calculated automatically by the software. A data set was generated containing sample names, peak intensity, and retention time and mass-to-charge ratio (m/z). Peak normalization to total area for each sample was used to eliminate the variations caused by the different volume of individual urine sample. The data was analyzed with SIMCA software and nonparametric Wilcoxon-Mann-Whitney test was used to verify those significantly altered variables selected by multivariate statistical models. Metabolites obtained from UPLC-QTOFMS analysis were identified with the aid of commercially obtained reference standards and web-based resources such as the Human Metabolome Database (www.hmdb.ca). The methods of analyzing the GC-MS raw data for the urine sample study were consistent with those set forth in Examples 1 and 3.

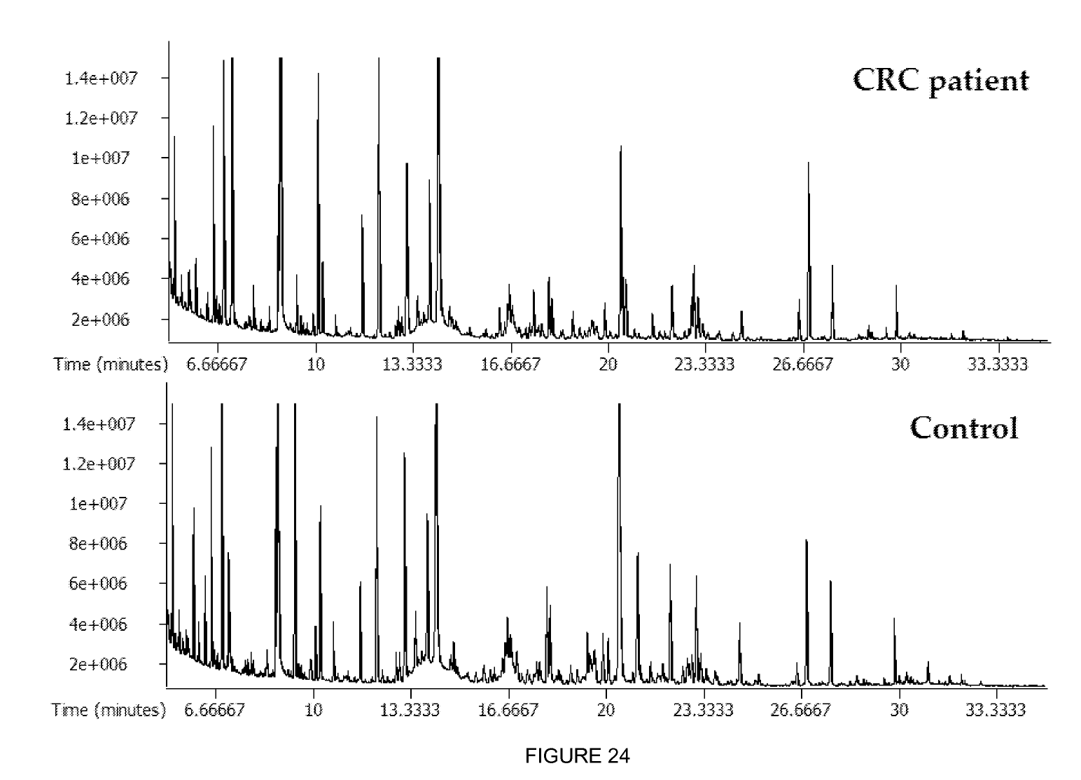

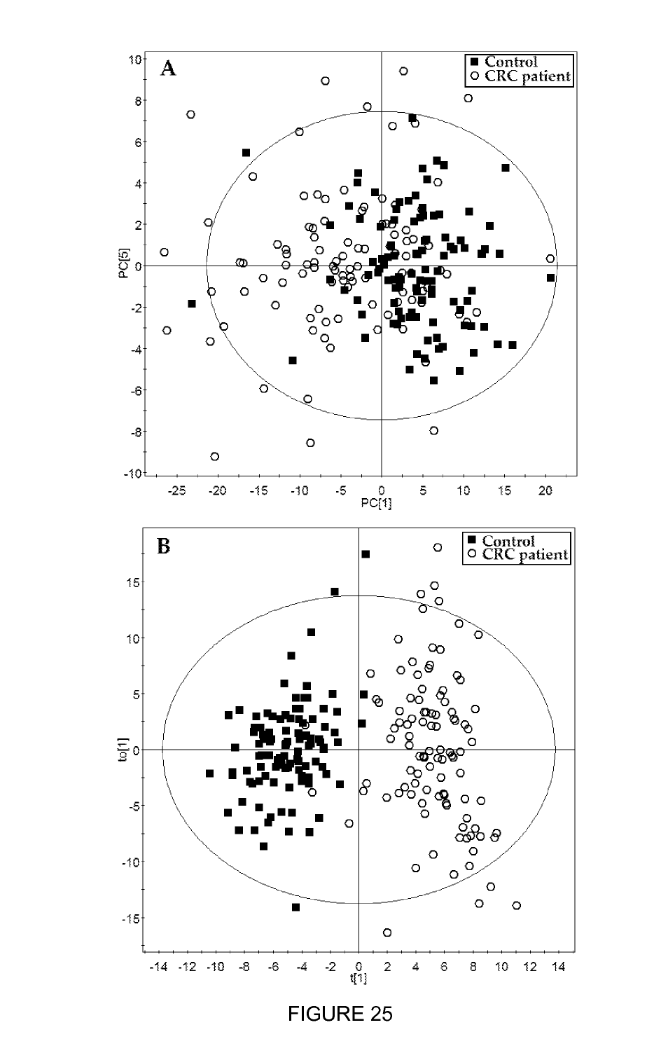

GC-MS analysis. One sample was excluded from the analysis during data analysis because of failed derivatization. Typical TIC chromatograms of urine samples from one cancer patient and one healthy control are shown in FIG. 24. After removal of two internal standards, a total of 361 variables were obtained and used in the following analysis. The data was normalized to the sum of all the peaks within sample. The normalized data was input into SIMCA-p software (v12.0). A separation tendency can be seen from the scores plot (FIG. 25A, PC1 vs PC 5, 7 components, R2X=0.429, Q2=0.302). Clear separation was obtained from the OPLS-DA model as shown in FIG. 25B, with only one CRC patient mixed into control samples (one prediction component with 2 orthogonal components, R2X=0.292, R2Y=0.825, Q2=0.735). These procedures are consistent with those set forth in Example 2.

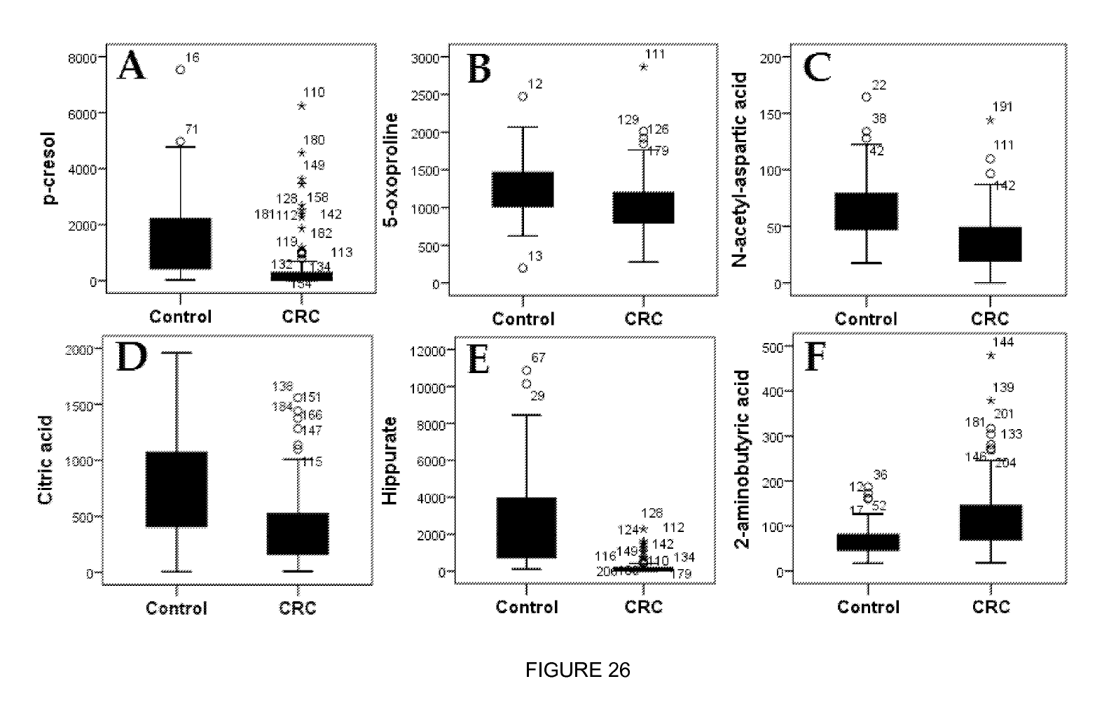

Wilcoxon-Mann-Whitney test statistical analysis. Based on VIP values from OPLS-DA model and the Wilcoxon-Mann-Whitney test, 151 differential variables were selected. A number of 66 metabolites were identified from those 151 differential variables, and 26 were verified by authentic standards (Table 16). The different expression levels of six typical metabolites in serum is shown in the box-plots of FIG. 26. This analysis is consistent with that set forth in Example 1.

TABLE 16
Differential urinary metabolites derived from OPLS-DA mode of GC-TOFMS
analysis with Wilcoxon-Mann-Whitney test

| | | Retention | | | | Chemical |
|---|---|---|---|---|---|---|
| No. | Metabolite^{a} | Time (min) | VIP^{C} | P^{d} | FC^{e} | Class |
| 1 | Ethanolamine | 5.27 | 1.73 | 1.92E−17 | 2.02 | Amine |
| 2 | Pyruvic acid^{b} | 5.52 | 2.07 | 1.06E−16 | −2.00 | Organic acid |
| 3 | Phenol^{b} | 5.67 | 1.49 | 2.50E−20 | −2.18 | Aromatic |
| | | | | | | compound |
| 4 | Hydroxyacetic acid^{b} | 5.90 | 2.20 | 1.56E−19 | −2.14 | Organic acid |
| 5 | p-Cresol^{b} | 7.10 | 1.54 | 5.89E−18 | −2.06 | Aromatic |
| | | | | | | compound |
| 6 | 2-methyl-3- | 7.14 | 1.04 | 1.08E−09 | 1.64 | Organic acid |
| | hydroxypropanoic | | | | | |
| | acid | | | | | |
| 7 | 2-aminobutyric acid^{b} | 7.31 | 1.53 | 1.34E−09 | 1.64 | Amino acid |
| 8 | 2-Hydroxyisocaproic | 8.19 | 1.44 | 2.70E−11 | 1.72 | Organic acid |
| | acid | | | | | |
| 9 | 4-hydroxybutanoic | 8.25 | 1.38 | 1.30E−10 | 1.69 | Organic acid |
| | acid^{b} | | | | | |
| 10 | 1,2,3- | 9.03 | 1.52 | 4.27E−15 | −1.92 | polyol |
| | trihydroxybutane | | | | | |
| 11 | Proline^{b} | 9.23 | 1.14 | 7.23E−08 | 1.55 | Amino acid |
| 12 | Uracil^{b} | 9.88 | 1.47 | 1.63E−10 | −1.69 | Nuclear acid |
| 13 | 2,3-dihydroxybutanoic | 10.07 | 1.66 | 7.61E−10 | 1.65 | Organic acid |
| | acid | | | | | |
| 14 | Fumarate^{b} | 10.11 | 1.43 | 2.63E−12 | 1.77 | Organic acid |
| 15 | 3-aminosalicylic acid | 10.45 | 1.04 | 8.75E−08 | −1.55 | Aromatic |
| | | | | | | compound |
| 16 | Methylmaleic acid | 11.02 | 1.25 | 6.42E−06 | 1.44 | Organic acid |
| 17 | Glutaric acid | 11.11 | 1.55 | 9.82E−14 | −1.85 | Organic acid |
| 18 | 2,4-dihydroxybutanoic | 11.18 | 1.81 | 4.78E−14 | −1.87 | Organic acid |
| | acid | | | | | |
| 19 | 5-aminopentanoic | 11.21 | 1.37 | 1.06E−09 | 1.64 | Amino acid |
| | acid | | | | | |
| 20 | 2-hydroxyundecanoic | 11.28 | 1.23 | 2.83E−06 | 1.46 | Organic acid |
| | acid | | | | | |
| 21 | 3-methyl-4- | 11.88 | 1.07 | 8.44E−03 | 1.23 | Organic acid |
| | hydroxybutyrate | | | | | |
| 22 | Threitol | 12.93 | 2.01 | 1.33E−18 | −2.10 | Polyol |
| 23 | 1-methylhistamine | 13.46 | 1.17 | 1.40E−06 | 1.48 | Amine |
| 24 | 5-oxoproline^{b} | 13.47 | 1.12 | 1.02E−06 | −1.49 | Amino acid |
| 25 | Threonic acid^{b} | 13.88 | 1.47 | 8.18E−10 | −1.65 | Organic acid |
| 26 | 2-hydroxyglutaric acid^{b} | 14.65 | 1.09 | 2.42E−06 | −1.47 | Organic acid |
| 27 | 2-oxopentanedioic | 14.68 | 1.50 | 2.84E−18 | −2.08 | Organic acid |
| | acid^{b} | | | | | |
| 28 | Guanidinosuccinic | 14.77 | 1.28 | 5.13E−10 | −1.66 | Organic acid |
| | acid^{b} | | | | | |
| 29 | 1,2- | 14.81 | 1.46 | 1.93E−10 | −1.69 | Aromatic |
| | dihydroxyphenylene | | | | | compound |
| 30 | 3-methyl-hexanedioic | 15.36 | 1.04 | 2.70E−07 | −1.52 | Organic acid |
| | acid | | | | | |
| 31 | Ribofuranose | 15.55 | 1.09 | 1.29E−19 | −2.15 | Sugar |
| 32 | Ribose | 16.23 | 1.25 | 8.75E−08 | −1.55 | Sugar |
| 33 | Allantoin | 16.33 | 1.18 | 1.01E−14 | −1.90 | Nuclear acid |
| 34 | Erythro-pentonic acid | 16.54 | 1.60 | 2.57E−14 | −1.88 | Sugar |
| | | | | | | derivate |
| 35 | Xylose^{b} | 16.61 | 1.36 | 1.91E−07 | −1.53 | Sugar |
| 36 | Putrescine^{b} | 16.67 | 1.26 | 8.64E−08 | 1.54 | Polyamine |
| 37 | N-acetyl-aspartic acid | 16.77 | 1.73 | 2.26E−13 | −1.84 | Amino acid |
| 38 | Indoxyl sulfate | 17.71 | 1.82 | 7.20E−18 | −2.06 | Aromatic |
| | | | | | | compound |
| 39 | Arabitol^{b} | 17.96 | 1.34 | 2.24E−16 | −1.98 | Polyol |
| 40 | 5-hydroxyindole | 18.47 | 1.77 | 1.74E−13 | −1.84 | Aromatic |
| | | | | | | compound |
| 41 | Orotic acid^{b} | 18.64 | 1.17 | 7.78E−07 | −1.49 | Nuclear acid |
| 42 | Glycerolphosphate | 19.01 | 1.92 | 2.53E−17 | −2.03 | Lipid |
| 43 | 6-deoxyglucitol | 19.18 | 1.13 | 5.26E−07 | −1.50 | Sugar |
| | | | | | | derivate |
| 44 | Galactonic acid | 19.24 | 1.09 | 5.55E−17 | −2.02 | Sugar |
| | | | | | | derivate |
| 45 | Homovanillic acid^{b} | 19.26 | 2.03 | 2.02E−20 | −2.19 | Aromatic |
| | | | | | | compound |
| 46 | Glycerol, 2- | 19.41 | 1.80 | 1.73E−15 | −1.94 | Lipid |
| | phposphate | | | | | |
| 47 | Sorbose^{b} | 20.25 | 1.30 | 4.05E−10 | −1.67 | Sugar |
| 48 | Citric acid^{b} | 20.41 | 1.53 | 9.32E−10 | −1.65 | Organic acid |
| 49 | Normetanephrine | 20.73 | 1.18 | 1.33E−05 | −1.42 | Aromatic |
| | | | | | | compound |
| 50 | Hippuric acid^{b} | 21.03 | 2.12 | 1.82E−27 | −2.55 | Aromatic |
| | | | | | | compound |
| 51 | Myristic acid^{b} | 21.14 | 1.73 | 5.14E−17 | −2.02 | Fatty acid |
| 52 | Mannose | 21.74 | 1.53 | 4.45E−14 | −1.87 | Sugar |
| 53 | Vanilmandelate | 21.81 | 1.47 | 1.12E−09 | −1.65 | Aromatic |
| | | | | | | compound |
| 54 | Glucuronic acid^{b} | 23.06 | 1.66 | 6.54E−11 | −1.71 | Sugar |
| | | | | | | derivate |
| 55 | Galactitol | 23.19 | 1.00 | 2.52E−08 | −1.58 | Polyol |
| 56 | Ascorbic acid | 23.39 | 1.21 | 6.12E−10 | −1.66 | Vitamine |
| 57 | Pantothenic acid | 24.45 | 1.30 | 3.04E−09 | −1.62 | Organic acid |
| 58 | Galactaric acid | 24.77 | 1.04 | 1.64E−06 | −1.48 | Sugar |
| | | | | | | derivate |
| 59 | p-hydroxymandelic | 28.03 | 1.40 | 6.12E−10 | −1.66 | Aromatic |
| | acid | | | | | compound |
| 60 | Linolic acid^{b} | 28.60 | 1.23 | 2.61E−09 | −1.63 | Fatty acid |
| 61 | m-hydroxyhippurate | 28.79 | 1.26 | 2.10E−19 | −2.14 | Aromatic |
| | | | | | | compound |
| 62 | Guanine | 29.73 | 1.17 | 3.74E−04 | 1.33 | Nuclear acid |
| 63 | Glycerol- | 30.22 | 1.53 | 3.71E−12 | −1.77 | Lipid |
| | mannoheptonic acid | | | | | |
| 64 | Uridine | 30.25 | 1.07 | 1.57E−06 | −1.48 | Nuclear acid |
| 65 | Hydroxy proline | 30.98 | 1.32 | 3.40E−17 | −2.03 | Dipeptide |
| | dipeptide | | | | | |
| 66 | Matitol^{b} | 32.12 | 2.04 | 3.16E−21 | 2.20 | Polyol |

^{a}Metabolites are identified using available library databases;
^{b}verified by reference compounds.
^{c}Variable importance in the projection (VIP) was obtained from OPLS-DA with a threshold of 1.0.
^{d}P-value and fold change (FC) are calculated from nonparametric Wilcoxon-Mann-Whitney test.
^{e}FC with a positive value indicates a relatively higher concentration present in CRC patients while a negative value means a relatively lower concentration as compared to the healthy controls.

Student's t test statistical analysis. Based on p values from Student's t test (p<0.05), 220 differential variables were obtained. A number of 100 metabolites were identified from those 220 differential variables, and 43 were verified by authentic standards (Table 17). This analysis is consistent with that set forth in Example 1.

TABLE 17
Differential metabolites between CRC patients
and controls using Student's t test (p < 0.05)

| | | Retention | | | |
|---|---|---|---|---|---|
| | | Time | | | |
| No. | Metabolite^{a} | (min) | P^{c} | FC^{d} | Chemical Class |
| 1 | Ethanolamine | 5.27 | 3.07E−12 | 2.06 | Amine |
| 2 | Pyruvic acid^{b} | 5.52 | 7.31E−18 | −1.93 | Organic acid |
| 3 | Phenol^{b} | 5.67 | 2.79E−09 | −2.89 | Aromatic compound |
| 4 | Hydroxyacetic acid^{b} | 5.90 | 1.51E−20 | −1.80 | Organic acid |
| 5 | Alanine^{b} | 6.30 | 1.53E−04 | −1.33 | Amino acid |
| 6 | 2-oxo-3-methyl- | 6.38 | 2.59E−02 | 1.36 | Organic acid |
| | butanoic acid^{b} | | | | |
| 7 | p-Cresol^{b} | 7.10 | 7.69E−10 | −3.47 | Aromatic compound |
| 8 | 2-methyl-3- | 7.14 | 5.53E−05 | 4.45 | Organic acid |
| | hydroxypropanoic acid | | | | |
| 9 | 2-aminobutyric acid^{b} | 7.31 | 9.62E−10 | 1.76 | Amino acid |
| 10 | Glyceraldehyde^{b} | 7.77 | 9.67E−04 | −1.29 | Lipid |
| 11 | 2-ethyl-3- | 8.13 | 2.17E−03 | 1.74 | Organic acid |
| | hydroxypropionic acid | | | | |
| 12 | 2-Hydroxyisocaproic | 8.19 | 1.23E−08 | 1.81 | Organic acid |
| | acid | | | | |
| 13 | 4-hydroxybutanoic acid^{b} | 8.25 | 4.98E−08 | 1.78 | Organic acid |
| 14 | 1,2,3-trihydroxybutane | 9.03 | 1.46E−09 | −1.86 | polyol |
| 15 | Isoleucine^{b} | 9.12 | 5.20E−03 | 1.15 | Amino acid |
| 16 | Proline^{b} | 9.23 | 7.99E−06 | 1.97 | Amino acid |
| 17 | Methylsuccinic acid | 9.68 | 1.30E−03 | −1.27 | Organic acid |
| 18 | Uracil^{b} | 9.88 | 4.78E−09 | −1.55 | Nuclear acid |
| 19 | 2,3-dihydroxybutanoic | 10.07 | 2.74E−11 | 1.96 | Organic acid |
| | acid | | | | |
| 20 | Fumarate^{b} | 10.11 | 1.29E−08 | 1.76 | Organic acid |
| 21 | 5-hydroxyhexanoic acid | 10.19 | 4.07E−02 | 2.50 | Organic acid |
| 22 | 3-aminosalicylic acid | 10.45 | 5.57E−05 | −1.40 | Aromatic compound |
| 23 | Methylmaleic acid | 11.02 | 1.00E−06 | 1.38 | Organic acid |
| 24 | Glutaric acid | 11.11 | 6.60E−10 | −1.61 | Organic acid |
| 25 | 2,4-dihydroxybutanoic | 11.18 | 1.86E−13 | −1.42 | Organic acid |
| | acid | | | | |
| 26 | 5-aminopentanoic acid | 11.21 | 6.72E−08 | 1.65 | Amino acid |
| 27 | 2-hydroxyundecanoic | 11.28 | 1.53E−06 | 2.69 | Organic acid |
| | acid | | | | |
| 28 | 3-methyl-4- | 11.88 | 3.23E−05 | 2.03 | Organic acid |
| | hydroxybutyrate | | | | |
| 29 | Threitol | 12.93 | 8.73E−17 | −1.75 | Polyol |
| 30 | 1-methylhistamine | 13.46 | 4.74E−06 | 1.37 | Amine |
| 31 | 5-oxoproline^{b} | 13.47 | 1.25E−05 | −1.23 | Amino acid |
| 32 | Ribonic acid, c-lactone | 13.69 | 6.87E−03 | −1.33 | Amino acid |
| 33 | Threonic acid^{b} | 13.88 | 4.79E−09 | −1.32 | Organic acid |
| 34 | 2-hydroxyglutaric acid^{b} | 14.65 | 2.03E−05 | −1.42 | Organic acid |
| 35 | 2-oxopentanedioic acid^{b} | 14.68 | 2.50E−09 | −2.32 | Organic acid |
| 36 | Guanidinosuccinic acid^{b} | 14.77 | 4.90E−07 | −1.80 | Organic acid |
| 37 | 1,2-dihydroxyphenylene | 14.81 | 6.05E−09 | −1.79 | Aromatic compound |
| 38 | 3-methyl-hexanedioic | 15.36 | 5.06E−05 | −1.82 | Organic acid |
| | acid | | | | |
| 39 | Ribofuranose | 15.55 | 2.16E−05 | −4.17 | Sugar |
| 40 | Citrulline^{b} | 15.56 | 2.74E−02 | −1.34 | Amino acid |
| 41 | 5-hydroxy-2- | 15.64 | 1.17E−02 | −1.26 | Organic acid |
| | piperidinecarboxylic | | | | |
| | acid | | | | |
| 42 | 4,6-dioxoheptanoic acid | 15.66 | 8.21E−03 | −1.37 | Organic acid |
| 43 | 3-desoxypentitol | 15.74 | 1.68E−03 | −1.18 | Polyol |
| 44 | p-hydroxyphenylacetic | 16.17 | 9.57E−03 | −1.54 | Aromatic compound |
| | acid | | | | |
| 45 | Ribose | 16.23 | 9.94E−07 | −1.39 | Sugar |
| 46 | Allantoin | 16.33 | 4.06E−06 | −7.20 | Nuclear acid |
| 47 | 3,4,5- | 16.53 | 1.25E−02 | −1.32 | Organic acid |
| | trihydroxypentanoic | | | | |
| | acid | | | | |
| 48 | Erythro-pentonic acid | 16.54 | 1.69E−10 | −1.73 | Sugar derivate |
| 49 | Xylose^{b} | 16.61 | 7.78E−08 | −1.38 | Sugar |
| 50 | Putrescine^{b} | 16.67 | 6.67E−07 | 1.50 | Polyamine |
| 51 | 4-aminobutanoic acid^{b} | 16.72 | 2.50E−02 | 5.39 | Amino acid |
| 52 | N-acetyl-aspartic acid | 16.77 | 3.14E−12 | −1.74 | Amino acid |
| 53 | Asparagine^{b} | 16.83 | 4.68E−02 | 1.24 | Amino acid |
| 54 | 3-hydroxyhexanedioic | 17.16 | 1.19E−04 | 1.69 | Organic acid |
| | acid | | | | |
| 55 | Gluconic acid, lactone | 17.58 | 2.62E−02 | 12.10 | Sugar derivate |
| 56 | Indoxyl sulfate | 17.71 | 1.38E−13 | −2.95 | Aromatic compound |
| 57 | 4-methyl-1,2- | 17.76 | 1.57E−02 | −2.06 | polyol |
| | dihydroxypentane | | | | |
| 58 | Arabitol^{b} | 17.96 | 1.25E−07 | −1.74 | Polyol |
| 59 | Rhamnose | 18.07 | 3.34E−05 | −1.39 | Sugar |
| 60 | 5-hydroxyindole | 18.47 | 7.94E−13 | −3.17 | Aromatic compound |
| 61 | Orotic acid^{b} | 18.64 | 5.02E−06 | −1.39 | Nuclear acid |
| 62 | Glycerolphosphate | 19.01 | 3.04E−15 | −1.69 | Lipid |
| 63 | 6-deoxyglucitol | 19.18 | 9.73E−06 | −1.32 | Sugar derivate |
| 64 | Galactonic acid | 19.24 | 1.98E−05 | −2.25 | Sugar derivate |
| 65 | Homovanillic acid^{b} | 19.26 | 3.89E−17 | −1.90 | Aromatic compound |
| 66 | Glutamine^{b} | 19.33 | 3.15E−02 | −1.20 | Amino acid |
| 67 | Glycerol, 2-phposphate | 19.41 | 2.82E−13 | −1.64 | Lipid |
| 68 | 3-methyl-histidine | 19.75 | 1.48E−03 | −1.21 | Amino acid |
| 69 | Sorbose^{b} | 20.25 | 2.93E−07 | −1.63 | Sugar |
| 70 | Ornithine^{b} | 20.32 | 1.42E−02 | −1.69 | Amino acid |
| 71 | Citric acid^{b} | 20.41 | 1.09E−09 | −1.85 | Organic acid |
| 72 | Isocitric acid^{b} | 20.48 | 1.42E−02 | −1.57 | Organic acid |
| 73 | Normetanephrine | 20.73 | 3.83E−06 | −1.36 | Aromatic compound |
| 74 | Hippuric acid^{b} | 21.03 | 6.16E−19 | −10.36 | Aromatic compound |
| 75 | Myristic acid^{b} | 21.14 | 2.68E−12 | −2.91 | Fatty acid |
| 76 | Fructose^{b} | 21.59 | 2.65E−02 | −4.38 | Sugar |
| 77 | Mannose | 21.74 | 1.04E−09 | −1.64 | Sugar |
| 78 | Vanilmandelate | 21.81 | 5.59E−09 | −1.49 | Aromatic compound |
| 79 | Galactose | 22.30 | 2.15E−03 | −1.26 | Sugar |
| 80 | Glucosamine | 22.68 | 3.48E−02 | −1.19 | Sugar derivate |
| 81 | Lysine^{b} | 22.83 | 4.71E−02 | −1.49 | Amino acid |
| 82 | Glucuronic acid^{b} | 23.06 | 2.27E−11 | −1.61 | Sugar derivate |
| 83 | Galactitol | 23.19 | 1.03E−04 | −1.37 | Polyol |
| 84 | Tyrosine^{b} | 23.22 | 2.14E−02 | −1.16 | Amino acid |
| 85 | Ascorbic acid | 23.39 | 2.12E−06 | −1.53 | Vitamine |
| 86 | Pantothenic acid | 24.45 | 3.17E−07 | −1.91 | Organic acid |
| 87 | Galactaric acid | 24.77 | 5.29E−05 | −1.31 | Sugar derivate |
| 88 | Xanthine^{b} | 25.08 | 2.65E−02 | −1.18 | Nuclear acid |
| 89 | N-Acetyl glucosamine | 26.31 | 3.51E−04 | −1.40 | Sugar derivate |
| 90 | Myo-inositol^{b} | 26.51 | 6.13E−03 | −1.36 | Polyol |
| 91 | p-hydroxymandelic acid | 28.03 | 3.16E−08 | −1.54 | Aromatic compound |
| 92 | Linolic acid^{b} | 28.60 | 1.52E−06 | −1.42 | Fatty acid |
| 93 | m-hydroxyhippurate | 28.79 | 7.65E−07 | −3.15 | Aromatic compound |
| 94 | Guanine | 29.73 | 4.81E−06 | 5.76 | Nuclear acid |
| 95 | Pseudo uridine^{b} | 29.83 | 2.71E−04 | −1.18 | Nuclear acid |
| 96 | Glycerol- | 30.22 | 1.01E−09 | −1.77 | Lipid |
| | mannoheptonic acid | | | | |
| 97 | Uridine | 30.25 | 2.89E−05 | −1.32 | Nuclear acid |
| 98 | Hydroxy proline | 30.98 | 2.11E−07 | −2.86 | Dipeptide |
| | dipeptide | | | | |
| 99 | Adenosine^{b} | 31.82 | 1.47E−02 | 1.15 | Nuclear acid |
| 100 | Matitol^{b} | 32.12 | 2.47E−17 | 5.59 | Polyol |
| 101 | Homoveratric acid^{e} | 5.34 | 2.17E−14 | −2.32 | Aromatic compound |
| 102 | Trimethylamine N-oxide^{e} | 0.67 | 2.02E−09 | −2.82 | Amine |
| 103 | Pyridoxal^{e} | 1.37 | 9.60E−11 | −3.45 | Pyridoxal |
| 104 | Cholic acid^{e} | 7.96 | 5.69E−07 | −16.4 | Cholic acid |

^{a}Metabolites are identified using available library databases;
^{b}verified by reference compounds.
^{c}P-values are calculated from Student's t test.
^{d}Fold changes (FC) are calculated from mean values. FC with a positive value indicates a relatively higher concentration present in CRC patients while a negative value means a relatively lower concentration as compared to the healthy controls.
^{e}Metabolites detected in UPLS-QTOFMS system.

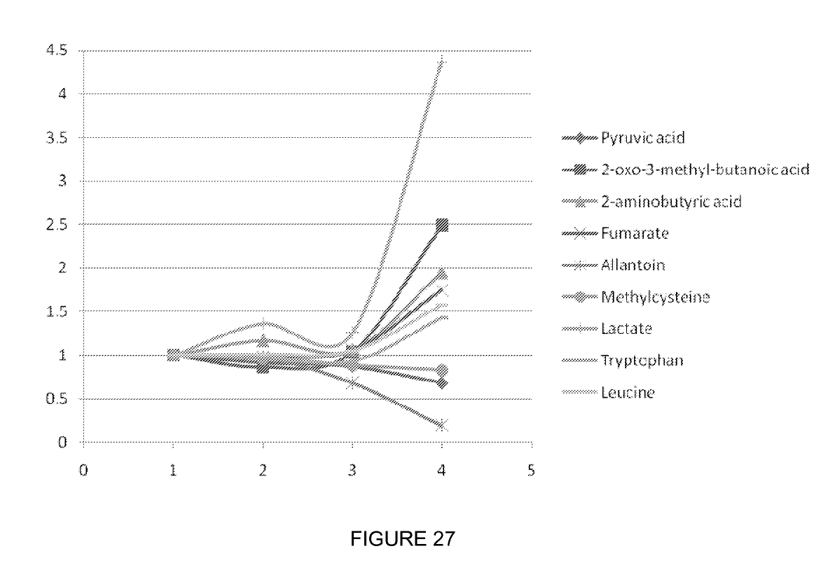

CRC Stage Differentiation of Markers. Some characteristic metabolites in certain TNM stage were shown in Table 18 and FIG. 27. Some metabolites showed consistent increase or decrease from stage I patients to stage IV patients, and some showed a dramatic increased in stage IV patients.

TABLE 18
Select metabolite alterations in different CRC stages.

| | Stage 1 | | Stage 2 | | Stage 3 | | Stage 4 | |
|---|---|---|---|---|---|---|---|---|
| Metabolite | mean | SEM^{a} | mean | SEM | mean | SEM | mean | SEM |
| Pyruvic acid | 1 | 0.53 | 0.92 | 0.45 | 0.87 | 0.47 | 0.68 | 0.41 |
| 2-oxo-3- | 1 | 0.65 | 0.86 | 0.47 | 1.03 | 0.77 | 2.49 | 4.29 |
| methyl- | | | | | | | | |
| butanoic acid | | | | | | | | |
| 2-aminobutyric | 1 | 0.38 | 1.17 | 0.72 | 1.06 | 0.59 | 1.95 | 1.96 |
| acid | | | | | | | | |
| Fumarate | 1 | 0.66 | 0.98 | 0.53 | 1.05 | 0.58 | 1.75 | 2.25 |
| Allantoin | 1 | 1.51 | 0.98 | 1.92 | 0.69 | 1.11 | 0.19 | 0.18 |
| Methylcysteine | 1 | 1.45 | 0.96 | 1.40 | 0.89 | 0.99 | 0.83 | 0.93 |
| Lactate | 1 | 1.02 | 1.36 | 1.63 | 1.26 | 1.48 | 4.36 | 8.04 |
| Tryptophan | 1 | 0.72 | 0.99 | 0.58 | 0.94 | 0.72 | 1.43 | 0.85 |
| Leucine | 1 | 0.72 | 1.01 | 0.87 | 1.05 | 0.86 | 1.58 | 1.43 |

^{a}Standard error of the mean.

### Conclusions

Thus, according to an embodiment of the invention, a GC-TOFMS-based urine metabolomics approach enabled identification of a number of metabolites that are differentially present in subjects with CRC as compared to healthy subjects (Table 19). Thus, in some aspects of the invention, GC-TOFMS can be used to detect metabolites as biomarkers for the diagnosis and/or prognosis of subjects with CRC. For example, the identified potential biomarkers showed lower levels of a number of aromatic compounds such as phenol, p-cresol, indoxyl sulfate, 5-hydroxyindole, homoyanillic acid, hippuric acid, vanilmanedelate, m-hydrorxyhippurate and homoyeratric acid. In addition, some fatty acids were detected at higher levels in the CRC patients (e.g., fumarate and methylmaleic acid), while others were detected at lower levels (e.g., pyruvic acid, 2-hydroxyglutaric acid and 2-oxopentanedioic acid). Some amino acids (e.g., 5-oxoproline and N-acetyl-aspartic acid)) were detected at lower levels in the serum of CRC patients, while 2-aminobutyric acid was observed at higher levels in CRC patients. Other differential metabolites were identified as well.

TABLE 19
Select differential urine metabolites

| | | Retention | | | | |
|---|---|---|---|---|---|---|
| No. | Metabolite^{a} | Time (min)^{f} | VIP^{C} | P^{d} | FC^{e} | Chemical Class |
| 1 | Pyruvic acid^{b} | 5.52 | 2.07 | 1.06E−16 | −2.00 | Organic acid |
| 2 | Phenol^{b} | 5.67 | 1.49 | 2.50E−20 | −2.18 | Aromatic |
| | | | | | | compound |
| 3 | p-cresol^{b} | 7.10 | 1.54 | 5.89E−18 | −2.06 | Aromatic |
| | | | | | | compound |
| 4 | 2-aminobutyric acid^{b} | 7.31 | 1.53 | 1.34E−09 | 1.64 | Amino acid |
| 5 | Uracil^{b} | 9.88 | 1.47 | 1.63E−10 | −1.69 | Nuclear acid |
| 6 | Fumarate^{b} | 10.11 | 1.43 | 2.63E−12 | 1.77 | Organic acid |
| 7 | Methylmaleic acid | 11.02 | 1.25 | 6.42E−06 | 1.44 | Organic acid |
| 8 | 5-oxoproline^{b} | 13.47 | 1.12 | 1.02E−06 | −1.49 | Amino acid |
| 9 | 2-hydroxyglutaric | 14.65 | 1.09 | 2.42E−06 | −1.47 | Organic acid |
| | acid^{b} | | | | | |
| 10 | 2-oxopentanedioic | 14.68 | 1.50 | 2.84E−18 | −2.08 | Organic acid |
| | acid^{b} | | | | | |
| 11 | N-acetyl-aspartic acid | 16.77 | 1.73 | 2.26E−13 | −1.84 | Amino acid |
| 12 | Indoxyl sulfate | 17.71 | 1.82 | 7.20E−18 | −2.06 | Aromatic |
| | | | | | | compound |
| 13 | 5-hydroxyindole | 18.47 | 1.77 | 1.74E−13 | −1.84 | Aromatic |
| | | | | | | compound |
| 14 | Homovanillic acid^{b} | 19.26 | 2.03 | 2.02E−20 | −2.19 | Aromatic |
| | | | | | | compound |
| 15 | Citric acid^{b} | 20.41 | 1.53 | 9.32E−10 | −1.65 | Organic acid |
| 16 | Hippuric acid^{b} | 21.03 | 2.12 | 1.82E−27 | −2.55 | Aromatic |
| | | | | | | compound |
| 17 | Myristic acid^{b} | 21.14 | 1.73 | 5.14E−17 | −2.02 | Fatty acid |
| 18 | Vanilmandelate | 21.81 | 1.47 | 1.12E−09 | −1.65 | Aromatic |
| | | | | | | compound |
| 19 | m-hydroxyhippurate | 28.79 | 1.26 | 2.10E−19 | −2.14 | Aromatic |
| | | | | | | compound |
| 20 | Guanine | 29.73 | 1.17 | 3.74E−04 | 1.33 | Nuclear acid |
| 21 | Matitol^{b} | 32.12 | 2.04 | 3.16E−21 | 2.20 | Polyol |
| 22 | Homoveratric acid | 5.34 (LC) | 2.05 | 2.17E−14 | −2.32 | Aromatic |
| | | | | | | compound |
| 23 | Trimethylamine N- | 0.67 (LC) | 1.68 | 2.02E−09 | −2.82 | Amine |
| | oxide | | | | | |
| 24 | Pyridoxal | 1.37 (LC) | 1.79 | 9.60E−11 | −3.45 | Pyridoxal |
| 25 | Cholic acid | 7.96 (LC) | 1.43 | 5.69E−07 | −16.4 | Cholic acid |

^{a}Metabolites are identified using available library databases;
^{b}verified by reference compounds.
^{c}Variable importance in the projection (VIP) was obtained from OPLS-DA with a threshold of 1.0.
^{d}P-value and fold change (FC) are calculated from nonparametric Wilcoxon-Mann-Whitney test.
^{e}FC with a positive value indicates a relatively higher concentration present in CRC patients while a negative value means a relatively lower concentration as compared to the healthy ontrols.
^{f}all other metabolites were detected by GC-TOFMS except those marker with LC, which were detected by UPLC-QTOFMS.

### Example 5

### Kit and Uses Thereof

### Kits

Kits of the invention include known amounts of one or more reference metabolites in the metabolite profile for colorectal cancer (CRC) to be assessed. The kit may include a single mixture of all the reference metabolites to be assessed, or may include a separate amount of each reference metabolite. The amounts of each reference metabolite in the metabolite profile to be assessed can be measured and used for comparison to the corresponding amount of the same metabolites in a sample from a subject. Each reference metabolite may be in solid form or in liquid form in the distributed kits. If the reference metabolites are in solid form, they are to be suspended into solution prior to use of the kit. Kits generally comprise at least one reference metabolite selected from the metabolites listed in Tables 2-4, 6-10, 12-14 and 16-19.

Kits generally include at least one container configured to contain the reference metabolites in the metabolite profile for CRC to be assessed. The container may be a tube, vial or multi-welled or multi-chambered plate. The container may have a single well or chamber, or the container may have multiple wells or chambers. For example, the container may be a multi-welled plate (e.g., a microtiter plate such as a 96-well microtiter plate). Other analogous containers are also appropriate. In some kits, the container may be appropriate for use in measurement of the reference metabolites and quantitation of the one or more metabolites to be assessed in a subject sample. In some kits, the container used for measurement of reference metabolites and quantitation of one or more metabolites in a subject sample is configured to be used for spectral analysis such as, for example, mass spectrometry and/or liquid chromatography. For example, the container may be configured for GC-TOFMS and/or LC-TOFMS. In other kits, the container may be configured for other analytical tests specific for one or more of the metabolites to be assessed in a subject sample (e.g., enzymatic, chemical, colorimetric, fluorometric, etc.). Examples of this type of kit are marketed by Biocrates Life Sciences (e.g., AbsoluteDQ Kits, SterolDQ Kits, etc.).

The container may be configured to hold a metabolite reference mixture, as set forth above, in one or more vials or tubes, or in one or more chambers or wells. Alternatively, the container may be configured to hold the reference amount of each metabolite to be assessed separately (e.g., one metabolite reference per chamber or well).

Some kits include a plurality of containers. For example, some kits include one or more containers having the reference metabolites. In addition, some kits include one or more containers having the reference metabolites and an additional container to be used in measurement of the reference metabolites and quantitation of the one or more metabolites to be assessed in a subject sample (e.g., a multi-welled plate or another tube or vial). In some kits, there is a single container that is used to contain the one or more reference metabolites and used in measurement of the reference metabolites and quantitation of the one or more metabolites to be assessed in a subject sample.

In kits where the container is a multi-welled or multi-chambered container, the reference amounts of the metabolites to be assessed may be located in one or more wells or chambers upon distribution of the kit for use. In some kits, the reference amounts of the metabolites to be assessed must be dispersed into one or more wells or chambers in using the kits.

The container of the kit can also be configured to accept a biological sample from at least one subject. For example, where the container of the kit includes multiple chambers or wells, a biological sample from a subject may be distributed into one or more chambers or wells. In some instances, one or more amounts of a subject sample may be distributed into a plurality of chambers or wells. The container of the kit is generally configured to accept fluid samples (e.g., fluid biological samples or solid biological samples that have been processed to obtain a fluid for analysis).

Some kits also include reagents useful for measurement of the reference metabolites and quantitation of the one or more metabolites to be assessed in a subject sample. These reagents may be included in the kit in one or more additional containers.

An exemplary kit may comprise a plurality of reference metabolites each provided in a separate container. The analytical container will be a microtiter plate configured for use with either a GC-MS or LC-MS device. The microtiter plate will have a sufficient number of wells to receive at least one The reference metabolites will have known concentrations and will be used to dispense a known amount of each reference metabolite into separate wells of the microtiter plate. After dispensing the reference metabolites into the analytical container, a portion of the subject sample can also be dispensed into the microtiter plate. Either a single portion of a subject sample is dispensed or a plurality of portions can be dispensed. If a plurality of portions is dispensed into the microtiter plate, each portion may be dispensed into a separate well. In addition, if a plurality of portions is dispensed into the microtiter plate, each portion may be of a different amount.

### Uses

Kits may be used to perform the methods of the invention to provide a diagnosis or prognosis for a subject having, or suspected of having, colorectal cancer (CRC) by enabling quantitation of the metabolites in a metabolite profile. For example, kits of the invention may be used to determine if a subject has CRC. In addition, kits of the invention may be used to determine a subject's prognosis or if a subject having CRC is responding to a treatment for CRC. Also, kits of the invention may be used to monitor a subject over time to determine if the subject develops CRC (e.g., a subject with an increased risk for CRC due to, e.g., environmental or genetic factors, or who previously had CRC that was treated into remission). Kits of the invention may also be used to determine what stage of CRC a subject has or what types of treatment may be appropriate for a subject (e.g., depending on what stage cancer a subject has). Depending on the intended use of a kit (e.g., diagnostic, prognostic, etc.), the kit may be used to assess different metabolite profiles comprising different selections of metabolites. For example, depending on the intended use of a kit, a kit may include at least one reference metabolite selected from the metabolites listed in Tables 2-4, 6-10, 12-14 and 16-19. In some kits, reference metabolites may be selected from Tables 6 9, 10, 16, 17 or 19. In some kits, reference metabolites may be selected from Tables 2-4, 9, 12, 13 or 15. In some kits, reference metabolites may be selected from Tables 4, 7, 14 and 18.

A biological sample obtained from a subject having, or suspected of having, CRC can be assessed using the kits of the invention. The sample may be a fluid sample (e.g., plasma, serum or urine) or may be a solid sample (e.g., tissue). Where a solid sample is obtained, it must be processed to obtain a fluid for analysis using a kit of the invention. In some uses of the kits, the metabolite profile in a subject sample may be assessed without processing of the sample. In other uses of the kits, the metabolite profile in a subject sample may require processing of the sample before being assessed.

A physician or medical technician may take a sample from a subject and send the sample to a clinical laboratory for testing using the kits of the invention. Alternatively, the physician or medical technician may be located at a clinical or medical facility that can perform testing using the kits of the invention.

The kits may be used to run a variety of tests to measure the amount of one or more metabolites in a subject sample. For example, the kits may be used to run a spectral analysis of a subject sample. Some kits are configured for spectral analyses such as gas chromatography and/or liquid chromatography. For example, a kit may be configured for GC-TOFMS and/or LC-TOFMS analysis of the metabolites of interest in a subject sample. Alternatively, kits may be configured so that analytical tests specific for different types of metabolites can be conducted (e.g., enzymatic, chemical, colorimetric, fluorometric, etc.) to measure the amount of the metabolites of interest in a subject's sample. In some uses, the reference metabolites included in the kit are used as positive controls for the analytical test performed to measure the amount of the metabolites of interest in a subject sample. In some uses, the reference metabolites included in the kit are used to help calibrate and/or measure the amount of the metabolites of interest in a subject sample. Depending on the type of analytical tests to be conducted to measure the metabolites of interest in a subject sample, different components used to conduct the analytical tests can be assembled into the kit with the one or more reference metabolites and the container.

The data obtained from the analytical tests performed using the kits is the amount of each of one or more metabolites of interest (i.e., metabolite profile) in a subject sample. This data can the be compared to reference metabolite levels in healthy subjects, or to subjects having colorectal cancer of different stages.

After the data from the analytical tests performed using the kit are obtained (i.e., metabolite profile for the subject sample (i.e., amount of each metabolite of interest)), the data can be inputted into a software program located on a computer terminal in the laboratory to generate a test result report, which can then be provided to the physician. The test result report may include information.

Once the physician receives the test result report from the clinical laboratory, the physician can evaluate the subject's colorectal cancer disease status. Based on the metabolite profile of the subject's sample assessed, which, as noted above, may be selected based on the subject's presentation to the physician, the test result report may indicate to the physician that the subject either does not have or does have colorectal cancer, that the subject is in remission or has had a recurrence of colorectal cancer, that the subject is responding to a particular treatment for colorectal cancer (e.g., surgical treatment, chemotherapeutic treatment, radiation treatment, etc.) and/or, if the subject has colorectal cancer, what stage of colorectal cancer the subject has. The physician can then, based on the colorectal cancer disease status indicated by the test result report, select an appropriate treatment for the subject, if necessary.

The examples and embodiments described herein are for illustrative purposes only. Various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of diagnosing colorectal cancer in a subject comprising:
(a) obtaining a sample from the subject;
(b) determining a metabolite profile for the subject's sample by measuring the amount of each of one or more metabolite biomarkers;
(c) comparing the subject's metabolite profile to a healthy control metabolite profile for the same one or more metabolite biomarkers; and
(d) identifying differences between the subject's metabolite profile and the healthy control metabolite profile;
wherein an increase or decrease in the level of one or more metabolite biomarkers in the subject's metabolite profile as compared to the healthy control metabolite profile indicates the presence of colorectal cancer in the subject.

2. The method of claim 1, wherein the one or more metabolite biomarkers have been previously identified as having increased or decreased levels in subjects having colorectal cancer.

3. The method of claim 1, wherein the healthy control metabolite profile comprises representative levels of the one or more metabolite biomarkers in healthy subjects.

4. The method of claim 1, wherein the healthy control metabolite biomarker profile comprises a metabolite profile for the subject obtained at a preceding time when the subject was known to be healthy.

5. The method of claim 1, wherein the one or more metabolite biomarkers comprises at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

6. The method of claim 1, wherein the sample from the subject is a bodily fluid.

7. The method of claim 1, wherein the sample from the subject comprises blood, saliva, serum or urine.

8. The method of claim 1, wherein determining the subject's metabolite profile comprises subjecting the subject's sample to spectral analysis.

9. The method of claim 8, wherein the spectral analysis comprises at least one of gas chromatography or mass spectrometry.

10. The method of claim 1, wherein the level of one or more metabolite biomarkers in the subject's metabolite profile indicates at least one appropriate method of treatment for the subject's colorectal cancer.

11. A method of determining the prognosis of a subject having colorectal cancer comprising:
(a) obtaining a sample from the subject after diagnosis of colorectal cancer;
(b) obtaining a sample from the subject after the subject has been treated for colorectal cancer;
(c) determining a metabolite profile for the subject's samples obtained in steps (a) and (b) by measuring the amount of each of one or more metabolite biomarkers in each sample;
(d) comparing the subject's metabolite profile before treatment to the subject's metabolite profile after treatment; and
(e) identifying differences between the subject's metabolite profile before and after treatment;
wherein an increase or decrease in the level of one or more metabolite biomarkers in the subject's metabolite profile after surgical treatment for colorectal cancer indicates the prognosis of the subject.

12. The method of claim 11, wherein the treatment for colorectal cancer is surgical treatment.

13. The method of claim 11, wherein the treatment for colorectal is chemotherapy or radiation therapy.

14. The method of claim 11, wherein the one or more metabolite biomarkers have been previously identified as having increased or decreased levels in subjects having colorectal cancer or in subjects with different stage colorectal cancers.

15. The method of claim 11, wherein the one or more metabolite biomarkers comprises at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

16. The method of claim 11, wherein the sample from the subject is a bodily fluid.

17. The method of claim 11, wherein the sample comprises blood, saliva, serum or urine.

18. The method of claim 11, wherein determining the subject's metabolite profile comprises subjecting the subject's sample to spectral analysis.

19. The method of claim 18, wherein the spectral analysis comprises at least one of gas chromatography or mass spectrometry.

20. The method of claim 11, wherein the subject's metabolite biomarker profile after treatment for colorectal cancer indicates decreased morbidity.

21. A method of identifying mechanisms of colorectal cancer pathogenesis comprising:
(a) obtaining samples from subjects diagnosed with colorectal cancer;
(b) obtaining samples from healthy subjects;
(c) determining the metabolite profile in each of the samples obtained in steps (a) and (b) by measuring the amount of one or more metabolite biomarkers in each sample;
(d) comparing the metabolite profile of subjects with colorectal cancer to the metabolite profile of healthy subjects;
(e) identifying one or more metabolite biomarkers that differentiate between subjects with colorectal cancer and healthy subjects, or between subjects with different stage colorectal cancer, wherein the one or more metabolite biomarkers are present in different amounts in healthy subject, subjects with colorectal cancer and/or subjects having different stages of colorectal cancer;
(f) identifying one or more cellular processes involving the one or more metabolite biomarkers, wherein the cellular processes comprise potential mechanisms of colorectal cancer pathogenesis.

22. The method of claim 21, wherein the metabolite profile of healthy subjects comprises a healthy control metabolite profile comprising one or more metabolite biomarkers having representative levels of the metabolite biomarkers in healthy subjects.

23. The method of claim 21, wherein the differentiating metabolite biomarkers comprise at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

24. The method of claim 21, wherein the samples comprise a bodily fluid.

25. The method of claim 21, wherein the samples comprise blood, saliva, serum or urine.

26. The method of claim 21, wherein determining the metabolite profiles comprises subjecting the samples to spectral analysis.

27. The method of claim 26, wherein the spectral analysis comprises at least one of gas chromatography or mass spectrometry.

28. The method of claim 21, wherein the level of one or more metabolite biomarkers in the subject's metabolite profile indicates at least one appropriate method of treatment for the subject's colorectal cancer.

29. The method of claim 21, wherein the one or more cellular processes comprising potential mechanisms of colorectal cancer pathogenesis indicates a desired method of treatment for colorectal cancer.

30. The method of claim 29, wherein selection of treatment for colorectal cancer comprises determining a method of treatment that will affect the one or more cellular processes comprising potential mechanisms of colorectal cancer pathogenesis.

31. A kit comprising:
(a) a plurality of reference metabolites, wherein the reference metabolites are known to be increased or decreased in subjects having colorectal cancer as compared to healthy subjects; and
(b) at least one container configured to hold the plurality of reference metabolites.

32. The kit of claim 31, wherein the plurality of reference metabolites comprises at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

33. The kit of claim 31, wherein each of the plurality of reference metabolites are in separate containers.

34. The kit of claim 31, comprising one or more known amounts of each of the plurality of reference metabolites.

35. The kit of claim 31, wherein the container comprises a multi-chambered container.

36. The kit of claim 31, wherein the container is configured to accept at least one biological sample from a subject.

37. The kit of claim 31, wherein the container is configured for spectral analysis of metabolites within the container.

38. The kit of claim 37, wherein the spectral analysis comprises at least one of gas chromatography or mass spectrometry.

39. A system comprising:
(a) one or more reference metabolites having differential levels in subjects based on colorectal cancer disease status;
(b) a analytical container configured to accept the one or more reference metabolites and at least one sample from at least one subject, wherein the at least one sample comprises at least one metabolite the same as at least one reference metabolite; and
(c) an analytical device configured to detect and/or measure the at least one reference metabolites and the at least one metabolite in the subject sample within the analytical container.

40. The system of claim 39, wherein the colorectal cancer disease status comprises the subject not having colorectal cancer, the subject having colorectal cancer or the subject having a particular stage of colorectal cancer.

41. The system of claim 39, wherein the one or more reference metabolites comprise at least one of oleamide, lysine, tryptophan, citruline, tyrosine, pyruvate, lactate, 2-hydroxybutrate, 3-hydroxybutrate, succinate, isocitrate, 5-hydroxytryptophan, 5-hydroxyindoleacetate, glutamate, 5-oxoproline, N-acetyl-aspartate, histidine, 3-methyl histidine, p-cresol, 2-hydroxyhippurate, phenylacetate, phenylacetylglutamine, p-hydroxyphenylacetate, pyruvic acid, 2-hydroxybutyric acid, 3-hydroxybutyric acid, 2-aminobutanoic acid, urea, isoleucine, succinic acid, fumarate, methylmaleic acid, malic acid, aspartic acid, 4-hydroxyproline, 2-oxo-pentanedioic acid, phenylalanine, asparagine, hypoxanthine, palmitic acid, linolic acid, oleic acid, octadecanoic acid, 2-hydroxybutyric acid, 3-hydroxybutyric, pyruvic acid, phenol, 2-aminobutyric acid, uracil, fumarate, methylmaleic acid, 2-hydroxyglutaric acid, 2-oxopentanedioic acid, indoxyl sulfate, 5-hydroxyindole, homovanillic acid, citric acid, hippuric acid, myristic acid, vanilmandelate, m-hydroxyhippurate, guanine, matitol, homoveratric acid, trimethylamine N-oxide, pyridoxal or cholic acid.

42. The system of claim 39, wherein the analytical device comprises a spectral analytical device.

43. The system of claim 39, wherein the analytical device comprises at least one of a mass spectrometer or a gas chromatographer.
